(19) United States
(12) Patent Application Publication
(10) Pub. No.: US 2024/0067980 A1
(43) Pub. Date: Feb. 29, 2024

(54) TRANSGENIC PLANTS WITH IMPROVED TRAITS

(71) Applicant: PLANTARC BIO LTD., Raanana (IL)

(72) Inventors: Dror SHALITIN, Raanana (IL); Noam GRIMBERG, Kibutz Lehavot Haviva (IL); Aviva KATZ, Kfar Saba (IL)

(21) Appl. No.: 18/381,155

(22) Filed: Oct. 17, 2023

Related U.S. Application Data
(63) Continuation of application No. PCT/IL2022/050404, filed on Apr. 19, 2022.
(60) Provisional application No. 63/178,171, filed on Apr. 22, 2021.

(51) Int. Cl. C12N 15/82; C12N 9/14

(52) U.S. Cl. CPC C12N 15/8261; C12N 9/14

(57) ABSTRACT

This disclosure relates to stay-green plants and plants with increased agricultural productivity, as well as to methods for obtaining same.

### CROSS REFERENCE TO RELATED APPLICATIONS

This application is a Bypass Continuation of PCT Patent Application No. PCT/IL2022/050404 having International filing date of Apr. 19, 2022, which claims the benefit of priority of U.S. Provisional Patent Application No. 63/178,171, filed Apr. 22, 2021, the contents of which are all incorporated herein by reference in their entirety.

### REFERENCE TO AN ELECTRONIC SEQUENCE LISTING

The contents of the electronic sequence listing (PAB-005-WOUS.xml; Size: 184,079 bytes; and Date of Creation: Oct. 27, 2023) is herein incorporated by reference in its entirety.

### FIELD OF INVENTION

This disclosure is broadly directed to plants having improved traits, in particular to transgenic plants having delayed senescence and/or increased biomass.

### BACKGROUND

World population is estimated to reach 9.2 billion in 2050. To sufficiently feed this population, the total food production will have to increase by 60%-70%. Climate models predict that warmer temperatures and increases in the frequency and duration of drought during the present century will have negative impact on agricultural productivity. For example, maize production in Africa could be at risk of significant yield losses as researchers predict that each degree-day that the crop spends above 30° C. reduces yields by 1% if the plants receive sufficient water. These predictions are similar to those reported for maize yield in the United States. It has been further shown that maize yields in Africa decreased by 1.7% for each degree-day the crop spent at temperatures of over 30° C. under drought. Wheat production in Russia decreased by almost one-third in 2010, largely due to the summer heat wave. Similarly, wheat production declined significantly in China and India in 2010, largely due to drought and sudden rise in temperature respectively, thereby causing forced maturity. These new global challenges require a more complex integrated agriculture.

Genetic engineering has the potential to address some of the most challenging biotic and abiotic constraints faced by farmers, which are not easily and adequately addressed through conventional plant breeding alone.

However, when genes coding for certain traits are transferred, typically from one plant species to another, the desired traits are not always achieved, due to lack of function, requirement for additional factors and the like. Moreover, breeding approaches are limited where polygenic traits are involved, time consuming and at times impossible to breed.

Therefore, there is a constantly growing need for genetically modified plants to express genes furnishing the plants with desired traits.

### SUMMARY

According to some aspects, there is provided a plant genetically modified to include a nucleic acid sequence encoding a RanGAP1 protein having a mutated, truncated or deleted WPP (Trp-Pro-Pro) motif and/or domain (pfam PF13943) or a mutated, truncated or deleted Leucine-Rich Repeat (LRR) domain. Each possibility and combination is a separate embodiment.

According to some aspects, there is provided a plant genetically modified to include a nucleic acid sequence encoding a RanGAP1 protein having a mutated, truncated or deleted WPP motif and/or domain (pfam PF13943) and a mutated, truncated or deleted Leucine-Rich Repeat (LRR) domain. Each possibility and combination is a separate embodiment.

According to some embodiments, the genetically modified RanGAP1 protein is gene edited version of the RanGAP1 endogenous to the plant. According to some embodiments, the endogenous RanGAP1 gene has been edited using gene-editing tools (e.g. by CRISPR/Cas technology, TALLEN, Zink-finger etc.) to obtain a nucleotide sequence encoding a RanGAP1 protein having a truncated, mutated or deleted WPP motif and/or domain and/or a mutated or deleted LRR domain.

According to some embodiments, the amino acid sequence of the genetically modified RanGAP1 has at least 70%, preferably at least 80%, more preferably at least 90%, at least 95%, at least 98% or 100% sequence homology to any one of SEQ ID NO: 12-19, 32-34, 39-42, 49-52, 59-63 or 71-75. According to some embodiments, the amino acid sequence of the genetically modified RanGAP1 has at least 70%, at least 80%, at least 90% at least 95%, at least 98% or 100% sequence homology to any one of SEQ ID NO: 12-19. According to some embodiments, the amino acid sequence of the genetically modified RanGAP1 has at least 70%, at least 80%, at least 90% at least 95%, at least 98% or 100% sequence homology to any one of SEQ ID NO: 32-34. According to some embodiments, the amino acid sequence of the genetically modified RanGAP1 has at least 70%, at least 80%, at least 90% at least 95%, at least 98% or 100% sequence homology to any one of SEQ ID NO: 39-42. According to some embodiments, the amino acid sequence of the genetically modified RanGAP1 has at least 70%, at least 80%, at least 90% at least 95%, at least 98% or 100% sequence homology to any one of SEQ ID NO: 49-52. According to some embodiments, the amino acid sequence of the genetically modified RanGAP1 has at least 70%, at least 80%, at least 90% at least 95%, at least 98% or 100% sequence homology to any one of SEQ ID NO: 59-63. According to some embodiments, the amino acid sequence of the genetically modified RanGAP1 has at least 70%, at least 80%, at least 90% at least 95%, at least 98% or 100% sequence homology to any one of SEQ ID NO: 71-75. Each possibility is a separate embodiment.

According to some embodiments, the amino acid sequence of the genetically modified RanGAP1 has at least 70%, at least 80%, at least 90% at least 95%, at least 98% or 100% sequence homology to any one of SEQ ID NO: 3, 8-10, 82-84. Each possibility is a separate embodiment.

According to some embodiments, the nucleotide sequence of the genetically modified RanGAP1 has at least 70%, at least 80%, at least 90% at least 95%, at least 98% or 100% sequence homology to any one of SEQ ID NO: 24-31 35-37, 44-47, 54-57, 65-69, 77-81 or 88. According to some embodiments, the nucleotide sequence of the genetically modified RanGAP1 has at least 70%, at least 80%, at least 90% at least 95%, at least 98% or 100% sequence homology to any one of SEQ ID NO: 24-31. According to some embodiments, the nucleotide sequence of the genetically modified RanGAP1 has at least 70%, at least 80%, at least 90% at least 95%, at least 98% or 100% sequence homology to any one of SEQ ID NO: 35-37. According to some embodiments, the nucleotide sequence of the genetically modified RanGAP1 has at least 70%, at least 80%, at least 90% at least 95%, at least 98% or 100% sequence homology to any one of SEQ ID NO: 44-47. According to some embodiments, the nucleotide sequence of the genetically modified RanGAP1 has at least 70%, at least 80%, at least 90% at least 95%, at least 98% or 100% sequence homology to any one of SEQ ID NO: 54-57. According to some embodiments, the nucleotide sequence of the genetically modified RanGAP1 has at least 70%, at least 80%, at least 90% at least 95%, at least 98% or 100% sequence homology to any one of SEQ ID NO: 65-69. According to some embodiments, the nucleotide sequence of the genetically modified RanGAP1 has at least 70%, at least 80%, at least 90% at least 95%, at least 98% or 100% sequence homology to any one of SEQ ID NO: 77-81. Each possibility is a separate embodiment.

According to some embodiments, the nucleotide sequence of the genetically modified RanGAP1 has at least 70%, at least 80%, at least 90% at least 95%, at least 98% or 100% sequence homology to any one of SEQ ID NO: 6, 20-22, 85-87. Each possibility is a separate embodiment.

According to some embodiments, the amino acid sequence of the genetically modified RanGAP1 has at least 70%, at least 80%, at least 90% at least 95%, at least 98% or 100% sequence homology to any one of SEQ ID NO: 14, 16, 17, 18, 19, 39, 49, 59, 71, 40, 50, 60, 72, 41, 51, 61, 73, 42, 52, 62, 74, 63 or 75. Each possibility is a separate embodiment.

According to some embodiments, the amino acid sequence of the genetically modified RanGAP1 has at least 70%, at least 80%, at least 90% at least 95%, at least 98% or 100% sequence homology to any one of SEQ ID NO:14, 16, 17, 39, 49, 59, 71, 40, 50, 60, 72, 41, 51, 61 or 73. Each possibility is a separate embodiment.

According to some aspects, the plant has been transformed to express a nucleic acid sequence encoding a protein having at least 70%, at least 75%, at least 80%, at least 85%, at least 90%, at least 95%, at least 96%, at least 97%, at least 98%, or at least 99% sequence homology to a plant RanGAP1 protein ortholog. Each possibility is a separate embodiment.

Advantageously, the expression of the RanGAP1 protein with the mutated, truncated or deleted WPP and/or LRR motifs and/or domains confers the plant with increased agricultural productivity comprising increased yield/biomass of the plant when grown under normal growth conditions, as compared to wild type plants

As further demonstrated hereinbelow, the plants disclosed herein are advantageously characterized by being significantly higher, significantly larger and/or having significantly larger leaves and/or roots, as compared to similar untransformed plants, without requiring additional use of fertilizers and/or watering.

In some embodiments, the RanGAP1 ortholog is derived from and/or resembles a potato (Solanum tuberosum) RanGAP1. According to some embodiments, the amino acid sequence of the genetically modified RanGAP1 has at least 70%, at least 80%, at least 90% at least 95%, at least 98% or 100% sequence homology to any one of SEQ ID NO: 12-19 or 32-34. Each possibility is a separate embodiment. According to some embodiments, the amino acid sequence of the genetically modified RanGAP1 has at least 70%, at least 80%, at least 90% at least 95%, at least 98% or 100% sequence homology to any one of SEQ ID NO: 14 or 16-19. Each possibility is a separate embodiment. According to some embodiments, the amino acid sequence of the genetically modified RanGAP1 has at least 70%, at least 80%, at least 90% at least 95%, at least 98% or 100% sequence homology to any one of SEQ ID NO: 14, 16 or 17. Each possibility is a separate embodiment. According to some embodiments, the nucleotide sequence of the genetically modified RanGAP1 has at least 70%, at least 80%, at least 90% at least 95%, at least 98% or 100% sequence homology to any one of SEQ ID NO: 24-31 or 35-37. Each possibility is a separate embodiment. According to some embodiments, the nucleotide sequence of the genetically modified RanGAP1 has at least 70%, at least 80%, at least 90% at least 95%, at least 98% or 100% sequence homology to any one of SEQ ID NO: 26 or 28-31. Each possibility is a separate embodiment. According to some embodiments, the nucleotide sequence of the genetically modified RanGAP1 has at least 70%, at least 80%, at least 90% at least 95%, at least 98% or 100% sequence homology to any one of SEQ ID NO: 26, 28 or 29. Each possibility is a separate embodiment.

In some embodiments, the RanGAP1 ortholog is derived from Camelina sativa and/or resembles the RanGAP1 of Camelina sativa. According to some embodiments, the amino acid sequence of the genetically modified RanGAP1 has at least 70%, at least 80%, at least 90% at least 95%, at least 98% or 100% sequence homology to any one of SEQ ID NO: 49-52. Each possibility is a separate embodiment. According to some embodiments, the amino acid sequence of the genetically modified RanGAP1 has at least 70%, at least 80%, at least 90% at least 95%, at least 98% or 100% sequence homology to any one of SEQ ID NO: 49-51. Each possibility is a separate embodiment. According to some embodiments, the nucleotide sequence of the genetically modified RanGAP1 has at least 70%, at least 80%, at least 90% at least 95%, at least 98% or 100% sequence homology to any one of SEQ ID NO: 54-57. Each possibility is a separate embodiment. According to some embodiments, the nucleotide sequence of the genetically modified RanGAP1 has at least 70%, at least 80%, at least 90% at least 95%, at least 98% or 100% sequence homology to any one of SEQ ID NO: 54-56. Each possibility is a separate embodiment.

In some embodiments, the RanGAP1 ortholog is derived from soy (Glycine Max) and/or resembles the RanGAP1 of Glycine Max. According to some embodiments, the amino acid sequence of the genetically modified RanGAP1 has at least 70%, at least 80%, at least 90% at least 95%, at least 98% or 100% sequence homology to any one of SEQ ID NO: 59-63. Each possibility is a separate embodiment. According to some embodiments, the amino acid sequence of the genetically modified RanGAP1 has at least 70%, at least 80%, at least 90% at least 95%, at least 98% or 100% sequence homology to any one of SEQ ID NO: 59-61. Each possibility is a separate embodiment. According to some embodiments, the nucleotide sequence of the genetically modified RanGAP1 has at least 70%, at least 80%, at least 90% at least 95%, at least 98% or 100% sequence homology to any one of SEQ ID NO: 65-69. Each possibility is a separate embodiment. According to some embodiments, the nucleotide sequence of the genetically modified RanGAP1 has at least 70%, at least 80%, at least 90% at least 95%, at least 98% or 100% sequence homology to any one of SEQ ID NO: 65-67. Each possibility is a separate embodiment.

In some embodiments, the RanGAP1 ortholog is derived from rice (Oryza sativa) and/or resembles the RanGAP1 of Oryza sativa. According to some embodiments, the amino acid sequence of the genetically modified RanGAP1 has at least 70%, at least 80%, at least 90% at least 95%, at least 98% or 100% sequence homology to any one of SEQ ID NO: 71-75. Each possibility is a separate embodiment. According to some embodiments, the amino acid sequence of the genetically modified RanGAP1 has at least 70%, at least 80%, at least 90% at least 95%, at least 98% or 100% sequence homology to any one of SEQ ID NO: 59-61. Each possibility is a separate embodiment. According to some embodiments, the nucleotide sequence of the genetically modified RanGAP1 has at least 70%, at least 80%, at least 90% at least 95%, at least 98% or 100% sequence homology to any one of SEQ ID NO: 77-81. Each possibility is a separate embodiment. According to some embodiments, the nucleotide sequence of the genetically modified RanGAP1 has at least 70%, at least 80%, at least 90% at least 95%, at least 98% or 100% sequence homology to any one of SEQ ID NO: 77-79. Each possibility is a separate embodiment.

In some embodiments, the RanGAP1 ortholog is derived from Arabidopsis thaliana and/or resembles the RanGAP1 of Arabidopsis thaliana. According to some embodiments, the amino acid sequence of the genetically modified RanGAP1 has at least 70%, at least 80%, at least 90% at least 95%, at least 98% or 100% sequence homology to any one of SEQ ID NO: 39-42. Each possibility is a separate embodiment. According to some embodiments, the amino acid sequence of the genetically modified RanGAP1 has at least 70%, at least 80%, at least 90% at least 95%, at least 98% or 100% sequence homology to any one of SEQ ID NO: 39-41. Each possibility is a separate embodiment. According to some embodiments, the nucleotide sequence of the genetically modified RanGAP1 has at least 70%, at least 80%, at least 90% at least 95%, at least 98% or 100% sequence homology to any one of SEQ ID NO: 44-47. Each possibility is a separate embodiment. According to some embodiments, the nucleotide sequence of the genetically modified RanGAP1 has at least 70%, at least 80%, at least 90% at least 95%, at least 98% or 100% sequence homology to any one of SEQ ID NO: 44-46. Each possibility is a separate embodiment. According to some embodiments, the nucleotide sequence of the genetically modified RanGAP1 has at least 70% sequence homology to any one of SEQ ID NO: 109-112. Each possibility is a separate embodiment. In some embodiments, the RanGAP1 ortholog is a mutated, truncated or WPP and/or LRR deleted version of the Arabidopsis thaliana RanGAP1, set forth in SEQ ID NO: 108.

In some embodiments, the RanGAP1 protein has at least 70%, at least 75%, at least 80%, at least 85%, at least 90%, at least 95%, at least 96%, at least 97%, at least 98%, or at least 99% sequence homology to any one of SEQ ID NO: 12, 14, 18 or 19. Each possibility is a separate embodiment. In some embodiments, the RanGAP1 protein has at least 70%, at least 75%, at least 80%, at least 85%, at least 90%, at least 95%, at least 96%, at least 97%, at least 98%, or at least 99% sequence homology to any one of SEQ ID NO: 13, 15, 16, or 17. Each possibility is a separate embodiment.

According to some embodiments, the amino acid sequence of the genetically modified RanGAP1 has at least 70%, at least 80%, at least 90% at least 95%, at least 98% or 100% sequence homology to any one of SEQ ID NO: 3, 8-10, 82-84. Each possibility is a separate embodiment.

In some embodiments, the RanGAP1 ortholog is a mutated, truncated or WPP and/or LRR deleted version of the peanut (Arachis hypogaea) RanGAP1, set forth in SEQ ID NO: 89. In some embodiments, the RanGAP1 ortholog is a mutated, truncated or WPP and/or LRR deleted version of the Rape seed (Canola) (Brassica napus) RanGAP1, set forth in SEQ ID NO: 90. In some embodiments, the RanGAP1 ortholog is a mutated, truncated or WPP and/or LRR deleted version of the Sugar beet (Beta vulgaris) RanGAP1, set forth in SEQ ID NO: 91. In some embodiments, the RanGAP1 ortholog is a mutated, truncated or WPP and/or LRR deleted version of the coffee (Coffea arabica) RanGAP1, set forth in SEQ ID NO: 92. In some embodiments, the RanGAP1 ortholog is a mutated, truncated or WPP and/or LRR deleted version of the carrot (Daucus carota) RanGAP1, set forth in SEQ ID NO: 93. In some embodiments, the RanGAP1 ortholog is a mutated, truncated or WPP and/or LRR deleted version of the Cotton (Gossypium hirsutum) RanGAP1, set forth in SEQ ID NO: 94. In some embodiments, the RanGAP1 ortholog is a mutated, truncated or WPP and/or LRR deleted version of the barley (Hordeum vulgare) RanGAP1, set forth in SEQ ID NO: 95. In some embodiments, the RanGAP1 ortholog is a mutated, truncated or WPP and/or LRR deleted version of the lettuce (Lactuca sativa) RanGAP1, set forth in SEQ ID NO: 96. In some embodiments, the RanGAP1 ortholog is a mutated, truncated or WPP and/or LRR deleted version of the cassava (Manihot esculenta) RanGAP1, set forth in SEQ ID NO: 97. In some embodiments, the RanGAP1 ortholog is a mutated, truncated or WPP and/or LRR deleted version of the Alfalfa (Medicago sativa) RanGAP1, set forth in SEQ ID NO: 98. In some embodiments, the RanGAP1 ortholog is a mutated, truncated or WPP and/or LRR deleted version of the Tobacco (Nicotania tabacum) RanGAP1, set forth in SEQ ID NO: 99. In some embodiments, the RanGAP1 ortholog is a mutated, truncated or WPP and/or LRR deleted version of the switchgrass (Panicum virgatum) RanGAP1, set forth in SEQ ID NO: 100. In some embodiments, the RanGAP1 ortholog is a mutated, truncated or WPP and/or LRR deleted version of the Sorghum (Sorghum bicolor) RanGAP1, set forth in SEQ ID NO: 101. In some embodiments, the RanGAP1 ortholog is a mutated, truncated or WPP and/or LRR deleted version of the tomato (Solanum lycopersicum) RanGAP1, set forth in SEQ ID NO: 102. In some embodiments, the RanGAP1 ortholog is a mutated, truncated or WPP and/or LRR deleted version of the wheat (Triticum aestivum) RanGAP1, set forth in SEQ ID NO: 103. In some embodiments, the RanGAP1 ortholog is a mutated, truncated or WPP and/or LRR deleted version of the cocoa (Theobroma cacao) RanGAP1, set forth in SEQ ID NO: 104. In some embodiments, the RanGAP1 ortholog is a mutated, truncated or WPP and/or LRR deleted version of the clover (Trifolium subterraneum) RanGAP1, set forth in SEQ ID NO: 105. In some embodiments, the RanGAP1 ortholog is a mutated, truncated or WPP and/or LRR deleted version of the cowpea (Vigna unguiculata) RanGAP1, set forth in SEQ ID NO: 106. In some embodiments, the RanGAP1 ortholog is a mutated, truncated or WPP and/or LRR deleted version of the Corn (Zea mays) RanGAP1, set forth in SEQ ID NO: 107. Each possibility is a separate embodiment.

According to some aspects, there is provided a plant genetically modified to express a nucleic acid sequence encoding a protein having at least 70%, at least 75%, at least 80%, at least 85%, at least 90%, at least 95%, at least 96%, at least 97%, at least 98%, or at least 99% sequence homology to a RanGAP1 ortholog protein. Each possibility is a separate embodiment.

According to some embodiments, the genetically modified RanGAP1 is at least modified to replace a conserved Tryptophan residue of the WPP motif and/or domain, corresponding to Trp 11 of the WPP motif (Trp-Pro-Pro) and/or domain (pfam PF13943) of potato RanGAP1 (SEQ ID NO: 11) with an alanine (e.g. SEQ ID NO: 12 and SEQ ID NO: 13) or other amino acid.

According to some embodiments, the genetically modified RanGAP1 is at least modified to replace at least one of two conserved residues at the LRR domain, corresponding to Arg190 and Asn219 of potato RanGAP1 (SEQ ID NO: 11) with an alanine or other amino acid (e.g. SEQ ID NO: 13 and SEQ ID NO: 15).

According to some embodiments, the genetically modified RanGAP1 is at least modified to delete/remove the WPP motif and/or domain (e.g. SEQ ID NO: 14 and SEQ ID NO: 15).

According to some embodiments, the genetically modified RanGAP1 is at least modified to provide a truncated LRR domain. According to some embodiments, the genetically modified RanGAP1 may have a LRR truncation producing a RanGAP1 protein corresponding to SEQ ID NO: 2 (e.g. SEQ ID NO: 16 and SEQ ID NO: 17 truncated before Leu295 and Glu308 respectively).

According to some embodiments, the genetically modified RanGAP1 may be truncated at the first methionine downstream to the WPP domain (e.g. SEQ ID NO: 18).

According to some embodiments, the genetically modified RanGAP1 may produce a nucleic acid sequence in which the START codon is replaced by other codon to produce a nucleotide sequence in which the first START codon is the first methionine downstream to the WPP motif (e.g. SEQ ID NO: 31).

According to some embodiments, the genetically modified RanGAP1 may include the acidic domain only (e.g. SEQ ID NO: 32-34).

According to some embodiments, the genetically modified RanGAP1 may be a chimera, e.g. be further modified to include a signal sequence and/or a protein and/or peptide enabling increased stability and/or solubility and/or facilitate cellular localization.

Advantageously, in some embodiments, the plant has an increased agricultural productivity when grown under normal growth conditions, as compared to its wild-type counterpart.

Advantageously, in some embodiments, the increased agricultural productivity of the plant comprises one or more of increasing a leaf area of the plant, increasing fresh weight of the plant, increasing dry weight of the plant, increasing fresh green organ weight, increasing shoot fresh weight (with and/or without roots), increasing flower numbers, increasing flower size, increasing fruit weight, increasing plant height, increasing root weight and volume, increasing the growth rate of the plant, increasing the seed yield, increasing the grain yield. Each possibility is a separate embodiment.

Additionally or alternatively, the plant may have an increased tolerance to drought and/or any abiotic stress.

In some embodiments, the RanGAP1 protein ortholog is derived from a plant.

In some embodiments, the RanGAP1 protein ortholog has a mutated WPP motif and/or domain. In some embodiments, the RanGAP1 protein ortholog lacks a WPP domain or part thereof (e.g. WPP motif). In some embodiments, the genetically modified plant has a mutated Leucine-Rich Repeat (LRR) domain. In some embodiments, the genetically modified plant lacks a LRR domain or part of the LRR domain.

In some embodiments, the RanGAP1 protein ortholog is derived from potato (Solanum tuberosum). According to some embodiments, the amino acid sequence of the genetically modified RanGAP1 has at least 70% sequence homology to any one of SEQ ID NO: 12-19 or 32-34. Each possibility is a separate embodiment. According to some embodiments, the amino acid sequence of the genetically modified RanGAP1 has at least 70% sequence homology to any one of SEQ ID NO: 14 or 16-19. Each possibility is a separate embodiment. According to some embodiments, the amino acid sequence of the genetically modified RanGAP1 has at least 70% sequence homology to any one of SEQ ID NO: 14, 16 or 17. Each possibility is a separate embodiment. According to some embodiments, the nucleotide sequence of the genetically modified RanGAP1 has at least 70% sequence homology to any one of SEQ ID NO: 24-31 or 35-37. Each possibility is a separate embodiment. According to some embodiments, the nucleotide sequence of the genetically modified RanGAP1 has at least 70% sequence homology to any one of SEQ ID NO: 26 or 28-31. Each possibility is a separate embodiment. According to some embodiments, the nucleotide sequence of the genetically modified RanGAP1 has at least 70% sequence homology to any one of SEQ ID NO: 26, 28 or 29. Each possibility is a separate embodiment.

In some embodiments, the RanGAP1 ortholog is derived from Arabidopsis thaliana and/or resembles the RanGAP1 of Arabidopsis thaliana. According to some embodiments, the amino acid sequence of the genetically modified RanGAP1 has at least 70% sequence homology to any one of SEQ ID NO: 39-42. Each possibility is a separate embodiment. According to some embodiments, the amino acid sequence of the genetically modified RanGAP1 has at least 70% sequence homology to any one of SEQ ID NO: 39-41. Each possibility is a separate embodiment. According to some embodiments, the nucleotide sequence of the genetically modified RanGAP1 has at least 70% sequence homology to any one of SEQ ID NO: 44-47. Each possibility is a separate embodiment. According to some embodiments, the nucleotide sequence of the genetically modified RanGAP1 has at least 70% sequence homology to any one of SEQ ID NO: 44-46. Each possibility is a separate embodiment. According to some embodiments, the nucleotide sequence of the genetically modified RanGAP1 has at least 70% sequence homology to any one of SEQ ID NO: 109-112. Each possibility is a separate embodiment. In some embodiments, the RanGAP1 ortholog is a mutated, truncated or WPP and/or LRR deleted version of the Arabidopsis thaliana RanGAP1, set forth in SEQ ID NO: 108.

In some embodiments, the RanGAP1 ortholog is derived from Camelina sativa and/or resembles the RanGAP1 of Camelina sativa. According to some embodiments, the amino acid sequence of the genetically modified RanGAP1 has at least 70% sequence homology to any one of SEQ ID NO: 49-52. Each possibility is a separate embodiment. According to some embodiments, the amino acid sequence of the genetically modified RanGAP1 has at least 70% sequence homology to any one of SEQ ID NO: 49-51. Each possibility is a separate embodiment. According to some embodiments, the nucleotide sequence of the genetically modified RanGAP1 has at least 70% sequence homology to any one of SEQ ID NO: 54-57. Each possibility is a separate embodiment. According to some embodiments, the nucleotide sequence of the genetically modified RanGAP1 has at least 70% sequence homology to any one of SEQ ID NO: 54-56. Each possibility is a separate embodiment.

In some embodiments, the RanGAP1 ortholog is derived from soy (Glycine Max) and/or resembles the RanGAP1 of Glycine Max. According to some embodiments, the amino acid sequence of the genetically modified RanGAP1 has at least 70% sequence homology to any one of SEQ ID NO: 59-63. Each possibility is a separate embodiment. According to some embodiments, the amino acid sequence of the genetically modified RanGAP1 has at least 70% sequence homology to any one of SEQ ID NO: 59-61. Each possibility is a separate embodiment. According to some embodiments, the nucleotide sequence of the genetically modified RanGAP1 has at least 70% sequence homology to any one of SEQ ID NO: 65-69. Each possibility is a separate embodiment. According to some embodiments, the nucleotide sequence of the genetically modified RanGAP1 has at least 70% sequence homology to any one of SEQ ID NO: 65-67. Each possibility is a separate embodiment.

In some embodiments, the RanGAP1 ortholog is derived from rice (Oryza sativa) and/or resembles the RanGAP1 of Oryza sativa. According to some embodiments, the amino acid sequence of the genetically modified RanGAP1 has at least 70% sequence homology to any one of SEQ ID NO: 71-75. Each possibility is a separate embodiment. According to some embodiments, the amino acid sequence of the genetically modified RanGAP1 has at least 70% sequence homology to any one of SEQ ID NO: 59-61. Each possibility is a separate embodiment. According to some embodiments, the nucleotide sequence of the genetically modified RanGAP1 has at least 70% sequence homology to any one of SEQ ID NO: 77-81. Each possibility is a separate embodiment. According to some embodiments, the nucleotide sequence of the genetically modified RanGAP1 has at least 70% sequence homology to any one of SEQ ID NO: 77-79. Each possibility is a separate embodiment.

In some embodiments, the RanGAP1 ortholog is a mutated, truncated or WPP and/or LRR deleted version of the peanut (Arachis hypogaea) RanGAP1, set forth in SEQ ID NO: 89. In some embodiments, the RanGAP1 ortholog is a mutated, truncated or WPP and/or LRR deleted version of the Rape seed (Canola) (Brassica napus) RanGAP1, set forth in SEQ ID NO: 90. In some embodiments, the RanGAP1 ortholog is a mutated, truncated or WPP and/or LRR deleted version of the Sugar beet (Beta vulgaris) RanGAP1, set forth in SEQ ID NO: 91. In some embodiments, the RanGAP1 ortholog is a mutated, truncated or WPP and/or LRR deleted version of the coffee (Coffea arabica) RanGAP1, set forth in SEQ ID NO: 92. In some embodiments, the RanGAP1 ortholog is a mutated, truncated or WPP and/or LRR deleted version of the carrot (Daucus carota) RanGAP1, set forth in SEQ ID NO: 93. In some embodiments, the RanGAP1 ortholog is a mutated, truncated or WPP and/or LRR deleted version of the Cotton (Gossypium hirsutum) RanGAP1, set forth in SEQ ID NO: 94. In some embodiments, the RanGAP1 ortholog is a mutated, truncated or WPP and/or LRR deleted version of the barley (Hordeum vulgare) RanGAP1, set forth in SEQ ID NO: 95. In some embodiments, the RanGAP1 ortholog is a mutated, truncated or WPP and/or LRR deleted version of the lettuce (Lactuca sativa) RanGAP1, set forth in SEQ ID NO: 96. In some embodiments, the RanGAP1 ortholog is a mutated, truncated or WPP and/or LRR deleted version of the cassava (Manihot esculenta) RanGAP1, set forth in SEQ ID NO: 97. In some embodiments, the RanGAP1 ortholog is a mutated, truncated or WPP and/or LRR deleted version of the Alfalfa (Medicago sativa) RanGAP1, set forth in SEQ ID NO: 98. In some embodiments, the RanGAP1 ortholog is a mutated, truncated or WPP and/or LRR deleted version of the Tobacco (Nicotania tabacum) RanGAP1, set forth in SEQ ID NO: 99. In some embodiments, the RanGAP1 ortholog is a mutated, truncated or WPP and/or LRR deleted version of the switchgrass (Panicum virgatum) RanGAP1, set forth in SEQ ID NO: 100. In some embodiments, the RanGAP1 ortholog is a mutated, truncated or WPP and/or LRR deleted version of the Sorghum (Sorghum bicolor) RanGAP1, set forth in SEQ ID NO: 101. In some embodiments, the RanGAP1 ortholog is a mutated, truncated or WPP and/or LRR deleted version of the tomato (Solanum lycopersicum) RanGAP1, set forth in SEQ ID NO: 102. In some embodiments, the RanGAP1 ortholog is a mutated, truncated or WPP and/or LRR deleted version of the wheat (Triticum aestivum) RanGAP1, set forth in SEQ ID NO: 103. In some embodiments, the RanGAP1 ortholog is a mutated, truncated or WPP and/or LRR deleted version of the cocoa (Theobroma cacao) RanGAP1, set forth in SEQ ID NO: 104. In some embodiments, the RanGAP1 ortholog is a mutated, truncated or WPP and/or LRR deleted version of the clover (Trifolium subterraneum) RanGAP1, set forth in SEQ ID NO: 105. In some embodiments, the RanGAP1 ortholog is a mutated, truncated or WPP and/or LRR deleted version of the cowpea (Vigna unguiculata) RanGAP1, set forth in SEQ ID NO: 106. In some embodiments, the RanGAP1 ortholog is a mutated, truncated or WPP and/or LRR deleted version of the Corn (Zea mays) RanGAP1, set forth in SEQ ID NO: 107. Each possibility is a separate embodiment.

In some embodiments, the RanGAP1 protein has at least 70%, at least 75%, at least 80%, at least 85%, at least 90%, at least 95%, at least 96%, at least 97%, at least 98%, or at least 99% sequence homology to any one of SEQ ID NO: 11-19. Each possibility is a separate embodiment.

In some embodiments, the RanGAP1 ortholog is derived from a fungus.

In some embodiments, the genetically modified RanGAP1 protein has at least 70%, at least 75%, at least 80%, at least 85%, at least 90%, at least 95%, at least 96%, at least 97%, at least 98%, or at least 99% sequence homology to any one of SEQ ID NO: 2, 3, 8, 10 or 82-84. Each possibility is a separate embodiment.

In some embodiments, the RanGAP1 protein has at least 70%, at least 75%, at least 80%, at least 85%, at least 90%, at least 95%, at least 96%, at least 97%, at least 98%, or at least 99% sequence homology to any one of SEQ ID NO: 3, 8, 9, or 10. Each possibility is a separate embodiment.

In some embodiments, the genetically modified plant may additionally or alternatively express a nucleic acid sequence encoding a protein having at least 70%, at least 75%, at least 80%, at least 85%, at least 90%, at least 95%, at least 96%, at least 97%, at least 98%, or at least 99% sequence homology to a putative SRF-TF-domain-containing protein ortholog. Each possibility is a separate embodiment.

In some embodiments, the putative SRF-TF-domain-containing protein ortholog is derived from a fungus. In some embodiments, the fungus is of Aspergillus sp.

In some embodiments, the putative SRF-TF-domain-containing protein ortholog has at least 70%, at least 75%, at least 80%, at least 85%, at least 90%, at least 95%, at least 96%, at least 97%, at least 98%, or at least 99% sequence homology to the amino acid sequence set forth in SEQ ID NO: 1. Each possibility is a separate embodiment.

According to some aspects, there is provided a method for increasing agricultural productivity of a plant, the method comprising genetically modifying the plant to express a nucleic acid sequence encoding a protein having at least 70% sequence homology to a RanGAP1 protein having a mutated, truncated or deleted WPP and/or LRR motifs and/or domain(s), thereby advantageously increasing the agricultural productivity of the plant when grown under normal growth conditions, as compared to wild type plants.

According to some embodiments, there is provided a method for genetically modifying a plant to express a nucleic acid sequence encoding a RanGAP1 protein ortholog increasing the yield/biomass of the plant when grown under normal growth conditions, as compared to wild type plants, wherein increasing the yield of the plant comprises one or more increasing a leaf area of the plant, increasing fresh weight of the plant, increasing fresh green organ weight, increasing shoot fresh weight (with and/or without roots), increasing fruit weight, increasing grain yield, increasing plant height, increasing root weight and volume, increasing the growth rate of the plant. Each possibility is a separate embodiment.

In some embodiments, the RanGAP1 ortholog is derived from a fungus.

In some embodiments, the protein has at least 70% sequence homology to any one of SEQ ID NO: 2, 3, 8, or 10. Each possibility is a separate embodiment.

In some embodiments, the protein has at least 70% sequence homology to any one of SEQ ID NO: 2, 3, 8, 9 or 10. Each possibility is a separate embodiment.

In some embodiments, the RanGAP1 ortholog is derived from a plant.

In some embodiments, the RanGAP1 ortholog is derived from potato (Solanum tuberosum).

In some embodiments, the RanGAP1 protein has at least 70% sequence homology to any one of SEQ ID NO: 11-18. Each possibility is a separate embodiment.

In some embodiments, the RanGAP1 protein has at least 70% sequence homology to any one of SEQ ID NO: 11-19. Each possibility is a separate embodiment.

In some embodiments, the genetically modifying further comprises expressing a nucleic acid sequence encoding a protein having at least 70% sequence homology to a putative SRF-TF-domain-containing protein ortholog. In some embodiments, the genetically modifying further comprises genetically modifying the endogenous SRF-TF-domain-containing protein.

In some embodiments, the putative SRF-TF-domain-containing protein ortholog is derived from a fungus.

In some embodiments, the putative SRF-TF-domain-containing protein ortholog has at least 70% sequence homology to the amino acid sequence set forth in SEQ ID NO: 1. In some embodiments, the genetically modifying further comprises genetically modifying the endogenous SRF-TF-domain-containing protein to obtain a SRF-TF-domain-containing protein having at least 70%, at least 80%, at least 90%, at least 95% or 100% sequence homology to the amino acid sequence set forth in SEQ ID NO: 1. Each possibility is a separate embodiment.

In some embodiments, the nucleic acid is an exogenous nucleic acid. In some embodiments, the exogenous nucleic acid is stably integrated into the plant genome. In some embodiments, the nucleic acid is expressed in the plant via a vector.

According to some aspects, the vector comprises a nucleic acid encoding a protein having at least 70% sequence homology to RanGAP1 protein ortholog, advantageously, the expression of the protein from the nucleic acid confers an increase in agricultural productivity and/or growth rate of a plant or a photosynthetic eukaryotic organism, as compared to its wild-type counterpart.

In some embodiments, the RanGAP1 protein ortholog is derived from a plant.

In some embodiments, the RanGAP1 protein ortholog has a mutated WPP motif and/or domain. In some embodiments, the RanGAP1 protein ortholog lacks a WPP motif and/or domain or part thereof. In some embodiments, the genetically modified plant has a mutated Leucine-Rich Repeat (LRR) domain. In some embodiments, the genetically modified plant lacks a part of the LRR domain.

According to some embodiments, the amino acid sequence of the genetically modified RanGAP1 has at least 70%, at least 80%, at least 90% at least 95%, at least 98% or 100% sequence homology to any one of SEQ ID NO: 12-19, 32-34, 39-42, 49-52, 59-63, 71-75. According to some embodiments, the amino acid sequence of the genetically modified RanGAP1 has at least 70%, at least 80%, at least 90% at least 95%, at least 98% or 100% sequence homology to any one of SEQ ID NO: 12-19. According to some embodiments, the amino acid sequence of the genetically modified RanGAP1 has at least 70%, at least 80%, at least 90% at least 95%, at least 98% or 100% sequence homology to any one of SEQ ID NO: 32-34. According to some embodiments, the amino acid sequence of the genetically modified RanGAP1 has at least 70%, at least 80%, at least 90% at least 95%, at least 98% or 100% sequence homology to any one of SEQ ID NO: 39-42. According to some embodiments, the amino acid sequence of the genetically modified RanGAP1 has at least 70%, at least 80%, at least 90% at least 95%, at least 98% or 100% sequence homology to any one of SEQ ID NO: 49-52. According to some embodiments, the amino acid sequence of the genetically modified RanGAP1 has at least 70%, at least 80%, at least 90% at least 95%, at least 98% or 100% sequence homology to any one of SEQ ID NO: 59-63. According to some embodiments, the amino acid sequence of the genetically modified RanGAP1 has at least 70%, at least 80%, at least 90% at least 95%, at least 98% or 100% sequence homology to any one of SEQ ID NO: 71-75. Each possibility is a separate embodiment.

According to some embodiments, the nucleotide sequence of the genetically modified RanGAP1 has at least 70%, at least 80%, at least 90% at least 95%, at least 98% or 100% sequence homology to any one of SEQ ID NO: 24-31 35-37, 44-47, 54-57, 65-69, 77-81 or 88. According to some embodiments, the nucleotide sequence of the genetically modified RanGAP1 has at least 70%, at least 80%, at least 90% at least 95%, at least 98% or 100% sequence homology to any one of SEQ ID NO: 24-31. According to some embodiments, the nucleotide sequence of the genetically modified RanGAP1 has at least 70%, at least 80%, at least 90% at least 95%, at least 98% or 100% sequence homology to any one of SEQ ID NO: 35-37. According to some embodiments, the nucleotide sequence of the genetically modified RanGAP1 has at least 70%, at least 80%, at least 90% at least 95%, at least 98% or 100% sequence homology to any one of SEQ ID NO: 44-47. According to some embodiments, the nucleotide sequence of the genetically modified RanGAP1 has at least 70%, at least 80%, at least 90% at least 95%, at least 98% or 100% sequence homology to any one of SEQ ID NO: 54-57. According to some embodiments, the nucleotide sequence of the genetically modified RanGAP1 has at least 70%, at least 80%, at least 90% at least 95%, at least 98% or 100% sequence homology to any one of SEQ ID NO: 65-69. According to some embodiments, the nucleotide sequence of the genetically modified RanGAP1 has at least 70%, at least 80%, at least 90% at least 95%, at least 98% or 100% sequence homology to any one of SEQ ID NO: 77-81. Each possibility is a separate embodiment.

In some embodiments, the RanGAP1 ortholog protein has at least 70% sequence homology to the amino acid sequences set forth in any one of SEQ ID NO: 3, 8, 10, 12-18. Each possibility is a separate embodiment.

In some embodiments, the RanGAP1 ortholog protein has at least 70%, at least 80%, at least 90%, at least 95% or 100% sequence homology to the amino acid sequences set forth in any one of SEQ ID NO: 2, 3, 8, 10, 11-18. Each possibility is a separate embodiment.

According to some aspects, there is provided a plant comprising a nucleic acid sequence encoding a protein having at least 70% at least 80%, at least 90%, at least 95% or 100% sequence homology to a non-plant ortholog protein comprising a putative SRF-TF-domain, wherein the plant has prolonged stay-green trait as compared to its wild-type counterpart. Each possibility is a separate embodiment.

Advantageously, the transformed plant stays green, while its wild-type counter parts dry out and senesce, when grown under normal growth conditions as well as under drought conditions. As a result of the stay-green characteristic, plant's organs including but not limiting leaves, fruit and/or seed weight, obtained for the transformed plant, significantly exceed that of a similar plant not transformed with such genes.

As further demonstrated hereinbelow, the plants disclosed herein are advantageously characterized by being significantly higher, significantly larger and/or having significantly larger leaves and/or roots, as compared to similar untransformed plants, without requiring additional use of fertilizers and/or watering.

In some embodiments, the non-plant ortholog protein comprising a putative SRF-TF-domain is derived from a fungus.

In some embodiments, the non-plant ortholog protein comprising a putative SRF-TF-domain has at least 70% sequence homology to SEQ ID NO: 1.

According to some aspects, there is provided a method for conferring stay-green in a plant, the method comprising genetically modifying the plant to express a nucleic acid sequence encoding a protein having at least 70%, at least 80%, at least 90%, at least 95% or 100% sequence homology a non-plant ortholog protein having a putative SRF-TF-domain. Each possibility is a separate embodiment.

According to some embodiments, there is provided a method for genetically modifying a plant to express a nucleic acid sequence encoding a putative SRF-TF-domain protein increasing the yield/biomass of the plant when grown under normal growth conditions, as compared to wild type plants, wherein increasing the yield of the plant comprises one or more of increasing a leaf area of the plant, increasing fresh weight of the plant, increasing dry weight of the plant, increasing fresh green organ weight, increasing shoot fresh weight (with and/or without roots), increasing flower numbers, increasing flower size, increasing fruit weight, increasing plant height, increasing root weight and volume, increasing the growth rate of the plant, increasing the seed yield, increasing the grain yield. Each possibility is a separate embodiment, increasing fresh green organ weight. Each possibility is a separate embodiment.

In some embodiments, the putative SRF-TF-domain-containing protein is derived from a fungus.

In some embodiments, the putative SRF-TF-domain-containing protein has at least 70%, at least 80%, at least 90%, at least 95% or 100% sequence homology to SEQ ID NO: 1. Each possibility is a separate embodiment.

In some embodiments, the nucleic acid is an exogenous nucleic acid. In some embodiments, the exogenous nucleic acid is stably integrated into the plant genome. In some embodiments, the nucleic acid is expressed in the plant via a vector.

According to some aspects, there is provided a method for increasing the growth rate of a photosynthetic eukaryotic organism, the method comprising genetically modifying the photosynthetic eukaryotic organism to express a nucleic acid sequence encoding a protein having at least 70%, at least 75%, at least 80%, at least 85%, at least 90%, at least 95%, at least 96%, at least 97%, at least 98%, or at least 99% sequence homology to a RanGAP1 protein ortholog. Each possibility is a separate embodiment.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be described in relation to certain examples and embodiments with reference to the following illustrative figures so that it may be more fully understood.

FIG. 1A shows soybean plants transformed with the nucleic acid sequence set forth in SEQ ID NO: 7 to express the amino acid set forth in SEQ ID NO: 1, compared to wild-type control, grown in Israel.

FIG. 1B shows soybean plants transformed with the nucleic acid sequence set forth in SEQ ID NO: 7 to express the amino acid set forth in SEQ ID NO: 1, compared to wild-type control, grown in Wisconsin, US.

FIG. 1C shows photos of soybean plants transformed with the nucleic acid sequence set forth in SEQ ID NO: 7 to express the amino acid set forth in SEQ ID NO: 1, compared to wild-type control, grown in Israel.

FIG. 2A shows the average pod dry weight (white) and average seed dry weight (black) as obtained from two independent transformation events of soybeans (left and middle bars) transformed with the nucleic acid sequence set forth in SEQ ID NO: 7 to express the amino acid sequence set forth in SEQ ID NO: 1, as compared to control (right bars).

FIG. 2B shows the average and standard deviation of seed dry weight as obtained from two independent transformation events of soybeans plants transformed with the nucleic acid sequence set forth in SEQ ID NO: 7 (event 1 and Event 2) or SEQ ID NO: 6 (event 3 and Event 4) to express the amino acid sequence set forth in SEQ ID NO: 1 or SEQ ID NO: 3), respectively, as compared to control (left bar). Average and standard deviation of every 5 plots of each event are shown above bars as grams per plot.

FIG. 3 shows shoot fresh weight (gr) of tobacco plants transformed with the nucleic acid sequence set forth in SEQ ID NO: 4 to express the amino acid set forth in SEQ ID NO: 1, as compared to wild-type tobacco plants or tobacco plants transformed with GFP.

FIG. 4 shows fruit weight (gr) of tobacco plants transformed with the nucleic acid sequence set forth in SEQ ID NO: 4 to express the amino acid set forth in SEQ ID NO: 1, as compared to wild-type tobacco plants or tobacco plants transformed with GFP.

FIG. 5 shows rosette leaves weight (black), reproductive weight (vertical stripes) and total weight (horizontal stripes) of Arabidopsis thaliana plants transformed with the nucleic acid sequence set forth in SEQ ID NO: 4 to express the amino acid set forth in SEQ ID NO: 1, as compared to wild-type Arabidopsis thaliana plants.

FIG. 6A shows tobacco plants either wild-type or transformed with the nucleic acid sequence set forth in SEQ ID NO: 5 to express the amino acid set forth in SEQ ID NO: 2, grown in pots.

FIG. 6B shows tobacco plants either wild-type or transformed with the nucleic acid sequence set forth in SEQ ID NO: 5 to express the amino acid set forth in SEQ ID NO: 2, grown in fields in Israel.

FIG. 7 shows the average height (cm) of tobacco plants transformed with the nucleic acid sequence set forth in SEQ ID NO: 5 to express the amino acid set forth in SEQ ID NO: 2 (black), as compared to wild-type tobacco plants (white).

FIG. 8 shows the leaf area (cm 2) of tobacco plants transformed with the nucleic acid sequence set forth in SEQ ID NO: 5 to express the amino acid set forth in SEQ ID NO: 2 (black), as compared to wild-type tobacco plants (white).

FIG. 9 shows shoot fresh weight (gr) of tobacco plants transformed with the nucleic acid sequence set forth in SEQ ID NO: 5 to express the amino acid set forth in SEQ ID NO: 2, as compared to wild-type tobacco plants or tobacco plants transformed with GFP.

FIG. 10A shows roots of five independent tobacco plants transformed with the nucleic acid sequence set forth in SEQ ID NO: 5 to express the amino acid set forth in SEQ ID NO: 2 (lower lane), as compared to five independent tobacco plants transformed with GFP as control (upper lane).

FIG. 10B shows the average root weight (gr) of tobacco plants transformed with the nucleic acid sequence set forth in SEQ ID NO: 5 to express the amino acid set forth in SEQ ID NO: 2 (black), as compared to control plants transformed with GFP (white).

FIG. 11 shows rosette leaves weight (black), reproductive weight (vertical stripes) and total weight (horizontal stripes) of Arabidopsis thaliana plants transformed with the nucleic acid sequence set forth in SEQ ID NO: 5 to express the amino acid set forth in SEQ ID NO: 2, as compared to wild-type Arabidopsis thaliana plants.

FIG. 12 illustrates RanGAP1 protein domain composition of vertebrates, plants and fungi. The protein has a common Leucine-Rich Repeat (LRR) and Acidic Domains, while only plants have an additional WPP domain.

FIG. 13 illustrates fungal RanGAP1 transcript variants cloned to the binary vector. The variants originated from cDNA derived from environmental RNA by targeting the 3′-UTR and 5′-end of the fungal RanGAP1 mRNA using specific primers (SEQ ID NO 2, 8-10). The cloned DNA sequence encoding the amino acid sequence of SEQ ID NO: 2 was subjected to site-directed mutagenesis to replace the Glycine coding triad to a Methionine (ATG START codon) at its 5′-end yielding DNA sequences encoding the amino acid sequence of SEQ ID NO: 3.

FIG. 14 illustrates potato (Solanum tuberosum) RanGAP1 protein variants cloned to the binary vector. The cloned native DNA sequence of the potato RanGAP1 encoding the amino acid sequence of SEQ ID NO: 11 was subjected to modifications yielding DNA sequences encoding the amino acid sequence of SEQ ID NO: 12-19. Next to SEQ ID NOs, in brackets, are the variant type numbers.

FIG. 15 presents a bar chart of the best performing T3 generation events, evaluated for their average total weight using the described scoring method. Arabidopsis plants were transformed to express the DNA sequence encoding the RanGAP1 amino acid sequence of SEQ ID NO: 2-3, 8-10 for the fungi-derived RanGAP1 protein, and of SEQ ID NO: 11-17 for the potato-derived RanGAP1 protein.

FIG. 16 is a bar chart showing the fresh weight of Arabidopsis plants transformed to express a DNA sequence encoding RanGAP1 variants (with indicated sequences) derived from soy (prefix Gm), potato (prefix St), Arabidopsis (prefix At), or rice (prefix Os), compared to controls.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

For convenience, certain terms used in the specification, examples, and appended claims are collected here. Unless otherwise defined, all technical and scientific terms used herein have the same meaning as commonly understood by one of ordinary skill in the art to which this invention pertains.

There is provided herein, according to some aspects of the disclosure, a method for delaying senescence and/or conferring stay-green in a plant, the method comprising genetically modifying the plant to express a nucleic acid sequence encoding a protein having at least 60%, at least 70%, at least 75%, at least 80%, at least 85%, at least 90% or at least 95% sequence homology to SEQ ID NO: 1. Each possibility is a separate embodiment.

There is provided herein, according to other aspects of the disclosure, a method for delaying senescence and/or conferring stay-green in a plant, the method comprising genetically modifying the plant to express a nucleic acid sequence encoding a protein having at least 60%, at least 70%, at least 75%, at least 80%, at least 85%, at least 90% or at least 95% sequence homology to a putative SRF-TF-domain-containing protein derived from a fungus. Each possibility is a separate embodiment.

According to some embodiments, the fungus is from the genus Aspergillus. Members of the genus possess the ability to grow at high osmotic pressure (high concentration of sugar, salt, etc.). Commonly, the fungi grow on carbon-rich substrates, but many species of Aspergillus are capable of growing in nutrient-depleted environments, or environments with a complete lack of key nutrients.

That is, surprisingly, by expressing in the plant a protein obtained from Aspergillus furnishes the plant with a photosynthesis-associated trait, namely stay-green, despite its origin being a non-photosynthetic organism. Furthermore, SRF proteins include a MADS box has been shown to be involved in DNA-binding and dimerization and no indication suggesting involvement with photosynthesis or stay green ability has been suggested, and its involvement therewith thus entirely unexpected.

Maintaining/prolonging the greenness of the photosynthetic tissues of plants is a key strategy for increasing crop production under both normal and water-limited conditions.

The stay-green-phenotype can be categorized into four groups—types A-D. Type A is when leaves and stems prolong their photosynthetic activity, displaying a delay in senescence, however, once initiated, senescence proceeds at a usual rate. In type B senescence proceeds at a slower rate. In type C (cosmetic stay-green) the greenness is a result of chlorophyll-degradation-pathway failure while a normal rate of senescence with declining photosynthetic activity remains. In type D, the stay-green is due to high accumulation of chlorophyll in photosynthetic tissues that results in a delay in senescence but reduced ability in fixing CO₂.

All the above may improve agricultural yield and/or value of the plants. And as such maintaining/prolonging the greenness of the photosynthetic tissues of plants thus plays a key role in increasing crop production and/or crop value.

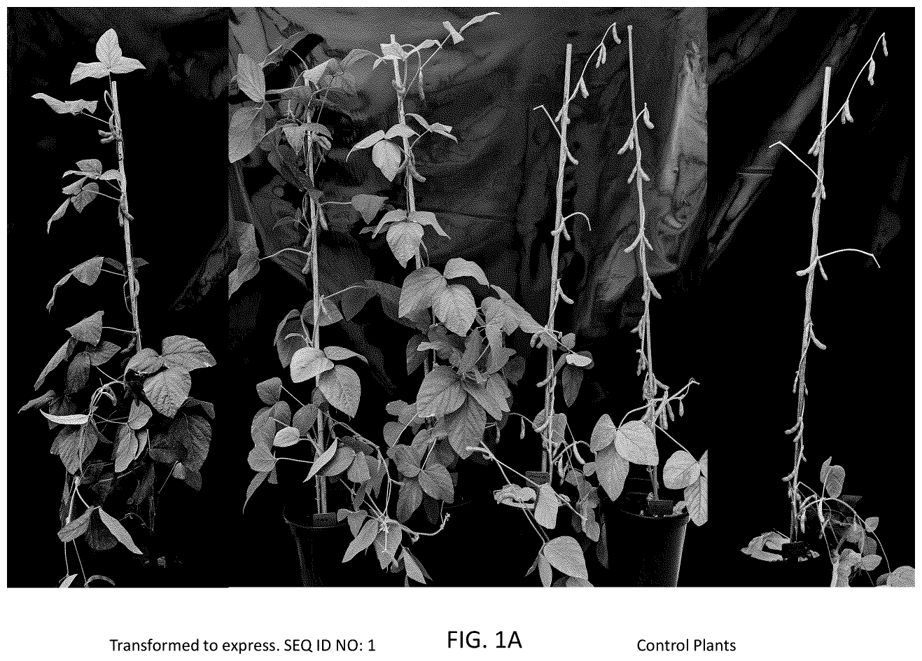

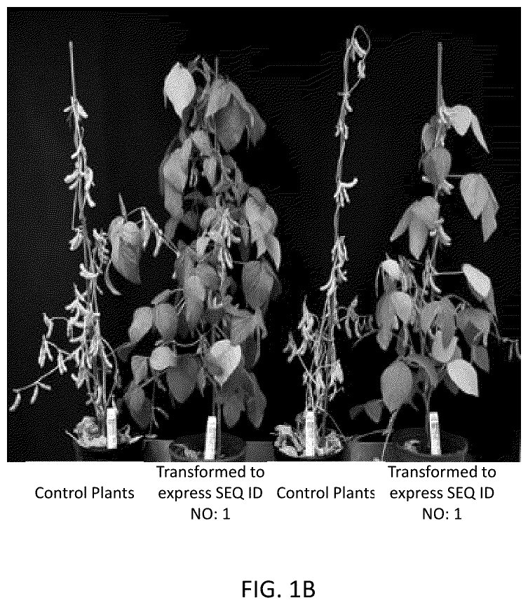

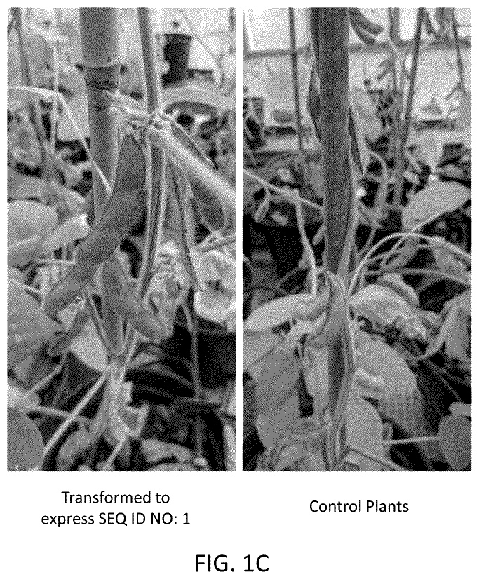

Advantageously, as demonstrated herein, the herein disclosed sequences provide/induce a stay-green effect that resembles type A or B, because the plants continue to accumulate photosynthates and have an increased biomass (see FIGS. 1c, 2-5).

According to some embodiments, there is provided a plant expressing a nucleic acid sequence encoding a protein having at least 80%, at least 85%, at least 90% or at least 95% sequence homology to SEQ ID NO: 1, wherein the plant is characterized by being stay-green. Each possibility is a separate embodiment.

According to some embodiments, there is provided a plant expressing a nucleic acid sequence encoding a protein having at least at least 60%, at least 65%, 70%, at least 75%, at least 80%, at least 85%, at least 90% or at least 95% sequence homology to a putative SRF-TF-domain-containing protein derived from a fungus, wherein the plant is characterized by being stay-green. Each possibility is a separate embodiment.

According to some embodiments, there is provided a plant seed comprising in its genome a nucleic acid sequence encoding a protein having at least 60%, at least 65%, at least 70%, at least 75%, at least 80%, at least 85%, at least 90% or at least 95% sequence homology to SEQ ID NO: 1, wherein the plant is characterized by being stay-green. Each possibility is a separate embodiment.

According to some embodiments, the nucleic acid sequence encoding a protein having at least 60%, at least 65%, at least 70%, at least 75%, at least 80% sequence homology to SEQ ID NO: 1, may have at least 70%, at least 75%, at least 80%, at least 85%, at least 90% or at least 95% sequence homology to the nucleic acid sequence set forth in SEQ ID NO: 4 or SEQ ID NO: 7.

According to some embodiments, the nucleic acid encoding the amino acid set forth in SEQ ID NO: 1 may be adapted according to the codon usage typical for the plant in which it is expressed.

According to some embodiments, there is provided a method for genetically modifying a plant to express a nucleic acid sequence encoding a protein having at least 60%, at least 65%, at least 70%, at least 75%, or at least 80% sequence homology to SEQ ID NO: 1 or SEQ ID NO: 2 or SEQ ID NO: 3, or SEQ ID NO: 8, or SEQ ID NO: 9, or SEQ ID NO: 10, or SEQ ID NO: 11, or SEQ ID NO: 12, or SEQ ID NO: 13, or SEQ ID NO: 14, or SEQ ID NO: 15, or SEQ ID NO: 16, or SEQ ID NO: 17, or SEQ ID NO: 18, or SEQ ID NO: 19 thereby increasing the agricultural productivity of the plant when grown under normal growth conditions, as compared to wild type plants, wherein increasing the agricultural productivity of the plant comprises one or more of increasing a leaf area of the plant, increasing fresh weight of the plant, increasing shoot fresh weight, increasing fruit weight, increasing plant height, increasing root weight, increasing the growth rate of the plant. Each possibility is a separate embodiment. Each possibility is a separate embodiment.

According to some embodiments, the nucleic acid sequence encoding a protein having at least 60%, at least 65%, at least 70%, at least 75%, at least 80% sequence homology to SEQ ID NO: 2, SEQ ID NO: 3, SEQ ID NO: 8 SEQ ID NO: 9 SEQ ID NO: 10 may have at least 70%, at least 75%, at least 80%, at least 85%, at least 90% or at least 95% sequence homology to the nucleic acid sequence set forth in SEQ ID NO: 5, SEQ ID NO: 20, SEQ ID NO: 21, SEQ ID NO: 22. According to some embodiments, the nucleic acid encoding the amino acid set forth in SEQ ID NO: 2 may be adapted according to the codon usage typical for the plant in which it is expressed. Each possibility is a separate embodiment.

According to some embodiments, the nucleic acid sequence encoding a protein having at least 60%, at least 65%, at least 70%, at least 75%, at least 80% sequence homology to SEQ ID NO: 3, may have at least 70%, at least 75%, at least 80%, at least 85%, at least 90% or at least 95% sequence homology to the nucleic acid sequence set forth in SEQ ID NO: 6. According to some embodiments, the nucleic acid encoding the amino acid set forth in SEQ ID NO: 2 may be adapted according to the codon usage typical for the plant in which it is expressed. Each possibility is a separate embodiment.

According to some embodiments, there is provided a method for genetically modifying a plant to express nucleic acid sequence encoding at least a portion of a protein having at least 60%, at least 65%, at least 70%, at least 75%, or at least 80% sequence homology to a putative SRF-TF-domain-containing protein derived from a fungus or to a RanGAP1 protein ortholog derived, for example, from a fungus or a plant, or to a mutated plant RanGAP1 protein ortholog lacks substantial parts of the WPP domain, thereby increasing the agricultural productivity of the plant when grown under normal growth conditions, as compared to wild type plants, wherein increasing the agricultural productivity of the plant comprises one or more of increasing a leaf area of the plant, increasing fresh weight of the plant, increasing shoot fresh weight, increasing fruit weight, increasing plant height, increasing root weight, increasing the growth rate of the plant. Each possibility is a separate embodiment.

That is, surprisingly, expressing in the plant a protein obtained from a fungus increases the agricultural productivity of the photosynthetic plant, despite its origin being a non-photosynthetic organism. Notwithstanding the aforesaid, it is equally unexpected and surprising that protein variants of plant orthologs of the RanGap1 gene that are mutated and/or lack substantial parts of the WPP domain, which is a unique domain exclusively present in plants' RanGap1 proteins, confer improved agricultural productivity. Even more, shorter protein variants that further lack parts of the Leucine-Rich Repeat (LRR) domain which is common and conserved across orthologs of all kingdoms were the most potent.

Moreover, RanGAP1 is a GTPase-activating protein, involved in the maintaining of the “ran gradient”, namely a high RanGDP concentrations in the cytosol, and a high RanGTP concentrations in the nucleus, which in turn provides the energy necessary for the transport of proteins into and out of the nucleus by karyopherin proteins.

That is, similarly to SRF-TF-protein, no indication suggesting involvement of RanGAP1, let alone a fungal-derived RanGAP1, in increasing agricultural productivity or biomass accumulation has been suggested, and its involvement therewith thus entirely unexpected.

In some embodiments, plants transformed with the nucleic acid encoding the RanGap1 proteins and variants thereof having amino acid sequences as set forth in SEQ ID NO: 2, 8, and 10 had increased weight, as compared with wild type plants, as indicated by the average total weight that includes fresh shoots (without roots) weight as measured as flowers developed, and fresh green organs weight as measured before the beginning of senescence.

In some embodiments, plants transformed to express the full-length fungal RanGap1 protein encoded by SEQ ID NO: 8 had significantly increased shoot weight compared to the partial gene variant encoded by SEQ ID NO: 2.

In some embodiments, plants transformed to express the full-length fungal RanGap1 protein encoded by SEQ ID NO: 8 had significantly increased shoot weight also compared to transform plants over-expressing the potato plant RanGAP1 encoded by SEQ ID NO: 11.

In some embodiments, plants transformed to express the fungal RanGap1 protein and variants thereof and specifically those having amino acid sequences as set forth in SEQ ID NO: 8 and SEQ ID NO: 10 have an improved ability to increase the total weight productivity of the Arabidopsis plant compared to wild-type plants.

In some embodiments, plants transformed to express the potato-derived RanGap1 protein and variants thereof, whose amino acid sequence is set forth in SEQ ID NO: 11-19 had increased weight, as compared with wild type plants, as indicated by the average total weight that includes fresh shoots (without roots) weight as measured as flowers developed, and fresh green organs weight as measured before the beginning of senescence.

In some embodiments, plants transformed to express the Potato-derived RanGap1 protein variants, having amino acid sequences as set forth in SEQ ID NO: 16-17 had significantly increased shoot weight compared to the protein having amino acid sequences as set forth in SEQ ID NO: 11.

In some embodiments, plants transformed to express the shorter potato-derived RanGap1 protein variants having amino acid sequences as set forth in SEQ ID NO: 14-17, increased the total weight productivity of the Arabidopsis plant vs. wild-type plants and plants transformed with RanGAP1 protein having amino acid sequences as set forth in SEQ ID NO: 11.

In some embodiments, the highest number of observed events of increase in total weight productivity was of plants transformed to express the shortest and most partial, potato-derived RanGap1 protein variants having the amino acid sequences set forth in SEQ ID NO: 16 and SEQ ID NO: 17.

That is, unexpected and surprising that protein variants of plant orthologs of the RanGap1 gene that are mutated and/or lack parts of the WPP domain (e.g. lack the WPP motif), which is a unique domain exclusively present in plants' RanGap1 proteins, confer improved agricultural productivity. Even more, shorter protein variants that further lack parts of the Leucine-Rich Repeat (LRR) domain which is common and conserved across orthologs of all kingdoms are the most potent in increasing the biomass of transformed plants.

According to some embodiments, there is provided a plant expressing a nucleic acid sequence encoding a protein having at least 60%, at least 65%, at least 70%, at least 75%, or at least 80% sequence homology to SEQ ID NO: 1 or SEQ ID NO: 2 or SEQ ID NO: 3, or SEQ ID NO: 8, or SEQ ID NO: 9, or SEQ ID NO: 10, or SEQ ID NO: 11, or SEQ ID NO: 12, or SEQ ID NO: 13, or SEQ ID NO: 14, or SEQ ID NO: 15, or SEQ ID NO: 16, or SEQ ID NO: 17, or SEQ ID NO: 18, or SEQ ID NO: 19, thereby increasing the agricultural productivity of the plant when grown under normal growth conditions, as compared to wild type plants, wherein increasing the yield of the plant comprises one or more of increasing a leaf area of the plant, increasing fresh weight of the plant, increasing shoot fresh weight, increasing fruit weight, increasing plant height, increasing root weight and volume, increasing dry weight of the plant, increasing the growth rate of the plant. Each possibility is a separate embodiment.

According to some embodiments, there is provided a plant seed expressing a nucleic acid sequence encoding a protein having at least 60%, at least 65%, at least 70%, at least 75%, or at least 80% sequence homology to SEQ ID NO: 1 or SEQ ID NO: 2 or SEQ ID NO: 3, or SEQ ID NO: 8, or SEQ ID NO: 9, or SEQ ID NO: 10, or SEQ ID NO: 11, or SEQ ID NO: 12, or SEQ ID NO: 13, or SEQ ID NO: 14, or SEQ ID NO: 15, or SEQ ID NO: 16, or SEQ ID NO: 17, or SEQ ID NO: 18, or SEQ ID NO: 19 thereby increasing the agricultural productivity of the plant when grown under normal growth conditions, as compared to wild type plants, wherein increasing the yield of the plant comprises one or more of increasing a leaf area of the plant, increasing fresh weight of the plant, increasing shoot fresh weight, increasing fruit weight, increasing plant height, increasing root weight and volume, increasing dry weight of the plant, increasing the growth rate of the plant. Each possibility is a separate embodiment.

According to some embodiments, the nucleic acid sequence encoding a protein having at least 60%, at least 65%, at least 70%, at least 75%, or at least 80% sequence homology to SEQ ID NO: 1, may have at least 60%, at least 65%, at least 70%, at least 75%, at least 80%, at least 85%, at least 90% or at least 95% sequence homology to the nucleic acid sequence set forth in SEQ ID NO: 4 or SEQ ID NO: 7. Each possibility is a separate embodiment.

According to some embodiments, the nucleic acid sequence encoding a protein having at least 80% sequence homology to SEQ ID NO: 2 or SEQ ID NO: 3, or SEQ ID NO: 8, or SEQ ID NO: 9, or SEQ ID NO: 10, or SEQ ID NO: 11, or SEQ ID NO: 12, or SEQ ID NO: 13, or SEQ ID NO: 14, or SEQ ID NO: 15, or SEQ ID NO: 16, or SEQ ID NO: 17, or SEQ ID NO: 18, or SEQ ID NO: 19 may have at least 70%, at least 75%, at least 80%, at least 85%, at least 90% or at least 95% sequence homology to the nucleic acid sequence set forth in SEQ ID NO: 5 or SEQ ID NO: 6 or SEQ ID NO: 20, or SEQ ID NO: 21, or SEQ ID NO: 22, or SEQ ID NO: 23, or SEQ ID NO: 24, or SEQ ID NO: 25, or SEQ ID NO: 26, or SEQ ID NO: 27, or SEQ ID NO: 28, or SEQ ID NO: 29, or SEQ ID NO: 30, or SEQ ID NO: 31. Each possibility is a separate embodiment.

According to some embodiments, the sequence may further furnish the plant with resistance to drought.

According to some embodiments, there is provided a method for increasing a growth rate of a photosynthetic organism, the method comprising genetically modifying the photosynthetic organisms to express a nucleic acid sequence encoding a protein having at least 60% sequence homology to SEQ ID NO: 2 or SEQ ID NO: 3, or SEQ ID NO: 8, or SEQ ID NO: 9, or SEQ ID NO: 10, or SEQ ID NO: 11, or SEQ ID NO: 12, or SEQ ID NO: 13, or SEQ ID NO: 14, or SEQ ID NO: 15, or SEQ ID NO: 16, or SEQ ID NO: 17, or SEQ ID NO: 18, or SEQ ID NO: 19 thereby increasing the growth rate of the photosynthetic eukaryotic organism. Each possibility is a separate embodiment.

According to some embodiments, the photosynthetic organisms may be an alga. According to some embodiments, the algae may be a microalga. According to some embodiments, the algae may be a macroalga.

As used herein, the term “stay-green” and “delayed senescence” may be used interchangeably and refer to mutant/transgenic plants or cultivars with the trait of maintaining their leaves for a longer period of time than the wild-type from which they are derived. According to some embodiments, stay-green plants have an extended duration of active photosynthesis.

As used herein, the terms “yield”, “crop-yield”, “agricultural productivity” and “agricultural output” may be used interchangeably and refer to the amount of a crop grown, or product such as wool, leaved, or extract produced, per unit area of land. According to some embodiments, the yield may be defined by “seed ratio” i.e. the ratio between the investment in seed versus the yield.

As used herein, the term “microalgae” refers to either prokaryotic or eukaryotic, mostly unicellular, microorganisms growing through photosynthesis.

As used herein, the term “microalgae” and “seaweed” refers to macroscopic, multicellular, marine algae. The term includes some types of Rhodophyta, Phaeophyta and Chlorophyta macroalgae.

As used herein, the term “growth rate” may refer to the rate at which the number of individuals, in a population, increases in a given time period, expressed as a fraction of the initial population.

According to some embodiments, the term “plant” as used herein, may be “Plantae sensu amplo” and may include plants in the widest sense e.g., including algae, lichen, fungi. According to some embodiments, the term “plant” may refer to green plants, also known as Viridiplantae, and may encompass organisms that have cellulose in their cell walls, possess chlorophylls a and b and have plastids bound by only two membranes that are capable of photosynthesis and of sugar production. According to some embodiments, the term “plant” may refer to plants in a broader sense and may include green plants as defined above plus the red algae (Rhodophyta), the glaucophyte algae (Glaucophyta) and Cyanobacteria.

The composition can be applied on any plant species, including, but not limited to, monocots and dicots. Examples of plant species include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), palm trees from the Arecaceae (e.g, coconut palm (Cocos mucifera), oil palm (Elaeis guineensis), date palm (Phoenix spp.), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentals), macadamia (Macadamia integrifolia), almond (Prunus amygdalus), Eucalyptus sp., Pinus spp., sugar beets (Beta vulgaris), sugarcane (Saccharum spp.), oats, barley, vegetables, ornamentals, lawn grasses (poaceae family) and conifers. Each possibility is a separate embodiment.

As used herein, the term “drought” refers to an event of shortages in water supply, whether atmospheric (below-average), surface water or ground or soil moisture or water. A drought can last for hours, days, months or years. According to some embodiments, drought may refer to at least 7 days with water shortage. According to some embodiments, drought may refer to any period of water shortage having a negative impact on the agricultural productivity of a crop.

As used herein, the term “genetically modified plants” may refer to plants transformed with a nucleic acid of foreign/exogenous origin which has been introduced into the genome of the plant by transformation with agrobacterium, biolistics, protoplasts, viral expression (transient) etc. as known in the art, as well as to endogenous nucleic acid of which, has been modified to resemble or be identical to a disclosed nucleic acid, using gene editing technologies, such as but not limited to CRISPR/Cas, TALLEN and zink-finger technologies.

As used herein, the terms “polypeptide”, “peptide”, and “protein” may be used interchangeably and refer to a polymer of amino acid residues. The terms apply to amino acid polymers in which one or more amino acid residues is an artificial chemical analogue of a corresponding naturally occurring amino acid, as well as to naturally occurring amino acid polymers. Polypeptides of the invention can be produced either from a nucleic acid disclosed herein, or by the use of standard molecular biology techniques or by artificial synthesis and methods. For example, a truncated protein of the invention can be produced by expression of a recombinant nucleic acid of the invention in an appropriate host cell, or alternatively by a combination of ex-vivo procedures, such as protease digestion and purification.

As used herein, “nucleic acid” includes reference to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to naturally occurring nucleotides.

Fragments and variants of the disclosed nucleotide sequences and proteins encoded thereby are also encompassed by the present invention. As used herein, “fragment” is intended to mean a portion of the nucleotide sequence or a portion of the amino acid sequence and hence protein encoded thereby. Fragments of a nucleotide sequence may encode protein fragments that retain the biological activity of the protein encoded. A fragment of a nucleotide sequence that encodes a biologically active portion of the protein of the invention will encode at least 15, 25, 30, 40, 50, 60, 70, 80, 90, 100, 110, 120, 150, 180, 200, 250, 300, 350 contiguous amino acids, or up to the total number of amino acids present in the full-length polypeptide of the invention. As used herein, “full-length sequence” in reference to a specified polynucleotide means having the entire nucleic acid sequence.

As used herein, the term “native sequence” and “endogenous sequence” may be used interchangeably and refer to the endogenous sequence, i.e., a non-engineered sequence found in the non-engineered plant.

As used herein, the terms “encoding” or “encoded” when used in the context of a specified nucleic acid mean that the nucleic acid comprises the requisite information to direct translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

“Variants” is intended to mean substantially similar sequences. For polynucleotides, a variant comprises a deletion and/or addition of one or more nucleotides at one or more internal sites within the reference polynucleotide and/or a substitution of one or more nucleotides at one or more sites in the disclosed polynucleotide. One of skill in the art will recognize that variants of the nucleic acids of the invention will be constructed such that the open reading frame is maintained. For polynucleotides, conservative variants include those sequences that, because of the degeneracy of the genetic code, encode the amino acid sequence of the polypeptides of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotide, such as those generated, for example, by using site-directed mutagenesis but which still encode the protein of the invention. Generally, variants of a particular polynucleotide of the invention will have at least about 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein. Each possibility is a separate embodiment.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, a polynucleotide that encodes a polypeptide with a given percent sequence identity to the polypeptides of SEQ ID NO: 1-3 are disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 60%, 65%, 70%, 75%, 80%, 85%, 90%, 91%, 92%, 93%, 94%, 95%, 96%, 97%, 98%, 99% or more sequence identity across the entirety of the disclosed sequences. Each possibility is a separate embodiment.

As used herein, the term “homology”, with reference to polynucleotide molecule, refers to a degree of sequence identity or similarity (homology) between nucleotide sequences indicative of shared ancestry. According to some embodiments a homolog may refer to a polynucleotide having substantially from about 70% to about 99% sequence identity, or more preferably from about 80% to about 99% sequence identity, or most preferable from about 90% to about 99% sequence identity, to about 99% sequence identity, to the referent nucleotide sequences of a referent polynucleotide molecule. Each possibility is a separate embodiment.

As used herein, the term “sequence identity”, “sequence similarity” and “sequence homology” may be used interchangeably to describe sequence relationships between two or more nucleotide sequences. The percentage of “sequence identity” between two sequences is determined by comparing two optimally aligned sequences. A sequence that is identical at every position in comparison to a reference sequence is said to be identical to the reference sequence and vice-versa. A first nucleotide sequence when observed in the 5′ to 3′ direction is said to be a “complement” of, or complementary to, a second or reference nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

According to some embodiments, the polypeptide may have exactly the sequence set forth in any of SEQ ID NOs: 1 and 2 and 3 and 8 and 9 and 10 and 11 and 12 and 13 and 14 and 15 and 16 and 17 and 18 and 19. Each possibility is a separate embodiment. According to some embodiments, the nucleic acid may have exactly the sequence set forth in any of SEQ ID NOs: 4 and 7 and 5 and 6 and 20 and 21 and 22 and 23 and 24 and 25 and 26 and 27 and 28 and 29 and 30 and 31. Each possibility is a separate embodiment.

According to some embodiments, the nucleic acid is an exogenous nucleic acid. According to some embodiments, the nucleic acid exogenous nucleic acid is stably integrated into the plant genome or expressed in the plants by other means such as via viral vectors etc.

According to some embodiments, the nucleic acid is a sequence endogenous to the plant, yet edited, e.g. via CRISPR-mediated gene editing, to resemble and/or be identical to the sequence set forth in a sequence listing, provided herein.

The article “a” and “an” are used herein to refer to one or more than one (i.e., to at least one) of the grammatical object of the article. By way of example, “a nucleic acid” means one or more nucleic acids. Throughout the specification the word “comprising,” or variations such as “comprises” will be understood to imply the inclusion of a stated element.

The following examples are included to demonstrate examples of certain preferred embodiments of the invention. It should be appreciated by those of skill in the art that the techniques disclosed in the examples that follow represent approaches the inventors have found function well in the practice of the invention, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

### Example 1—Stay-Green Soybean

Soybean plants (Glycine max cv. Williams82) were transformed with the nucleic acid set forth in SEQ ID NO: 7 to express the protein having the sequence set forth in SEQ ID NO: 1, under a constitutive 35S promoter. Four (4) independent events, single copy gene insert plants were grown for 3 generations (T3 plants) and checked for homozygosity and selected for further experiments.

These transgenic lines were grown in greenhouses under controlled growth conditions in 25(+−3) degrees Celsius and irrigated and fertilized as needed for 18 weeks either in Israel or in Wisconsin, US.

After 16 weeks, the plants were visually inspected. As seen from FIG. 1A (Israel) and FIG. 1B, (Wisconsin, US) different transformation events of William82 soybean plants stayed green, while wild type plants senescent normally (earlier).

Advantageously, as seen from FIG. 1C, not only the leaves stayed green, but also the soybean pods remained green and fresh in the plants transformed with the nucleic acid set forth in SEQ ID NO: 7 encoding the polypeptide having the amino acid sequence set forth in SEQ ID NO: 1.

### Example 2—Soybean with Increased Seed and Pod Weight

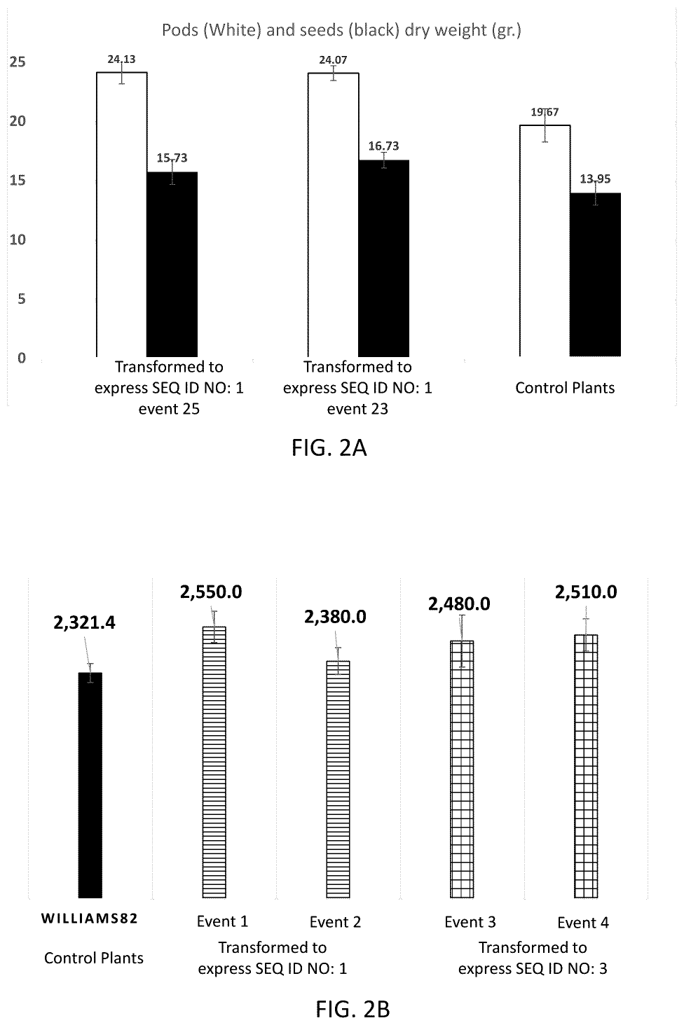

Soybean plants (Glycine max cv. Williams82) were transformed with the nucleic acid set forth in SEQ ID NO: 7 or SEQ ID NO 6 to express the protein having the sequence set forth in SEQ ID NO: 1 or SEQ ID NO: 3, under a constitutive 35S promoter, and grown under controlled conditions or under field conditions (FIG. 2).

Plants expressing SEQ ID NO:1 (FIG. 2A) were grown in Israel in 4-liter pots in a temperature-controlled greenhouse until plants matured and aged (18 weeks). The seed containing pods were dried and weighed, followed by weighing of the seeds alone. The weights were compared to those of wt Williams82 plants grown under the same conditions. FIG. 2A, shows that plants expressing the polypeptide set forth in SEQ ID NO: 1 yielded 22.6% more pods and 12.7-19.9% more seeds than the control plants, thus indicating that the stay-green phenotype is increasing agricultural yield type A or type B phenotype.

Plants expressing SEQ ID NO: 1 or SEQ ID NO: 3 (FIG. 2B), 2 independent events of each, were grown under field conditions in Stewardson Illinois, USA. Plants were grown in the field under the natural weather, temperatures, relative humidity, and rainfall occurring during the growing season of 2021-June to October 2021. The field was set that each soybean event or WT soybean (Williams82 variety) were placed as 5 repeats (plot), in 2-row plots, 20 feet long each row, with a total of 160 seeds for every plot. 8 rows of border surrounded the trial area. Each plot was harvested separately when seeds have dried to −13.5% humidity and weighed.

FIG. 2B, shows, SEQ ID NO:1, events 1 and 2, produced 9.8% and 2.5% more seed weight than control plants respectively. SEQ ID NO: 3, events 3 and 4, produced 6.8% and 8.1% more seed weight than control plants respectively.

### Example 3—Tobacco with Increased Fruit Weight and Shoot Fresh Weight

Tobacco plants (Nicotiana tabacum, cv. Little Dutch) were transformed with the nucleic acid set forth in SEQ ID NO: 4 to express the protein having the sequence set forth in SEQ ID NO: 1 under a constitutive 35S promoter. Five independent events, single copy gene insert plants were checked for homozygosity and selected for further experiments.

The plants were grown in the field under a net and drip irrigated and fertilized as needed for 16 weeks and the shoots/fruits were collected and weighted.

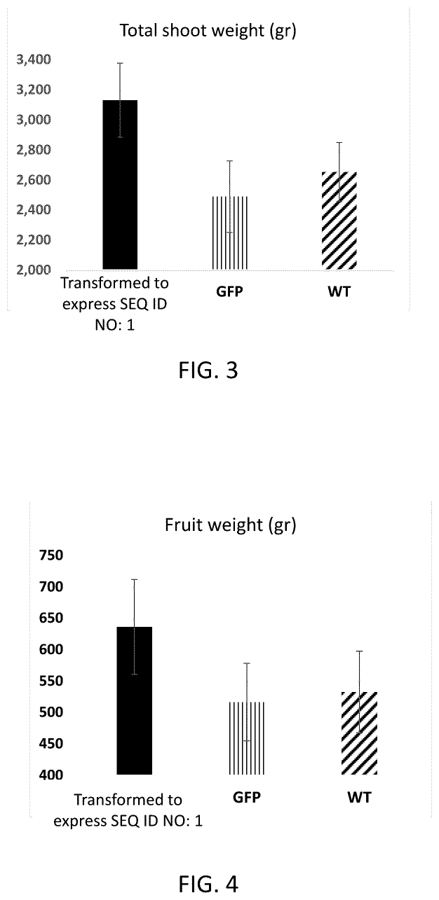

As seen from FIG. 3, the tobacco plants transformed with the herein disclosed nucleic acid encoding the protein set forth in SEQ ID NO: 1, had a significantly increased shoot weight, as compared to wild-type tobacco plants or tobacco plants transformed with GFP as control.

Furthermore, as seen from FIG. 4, the tobacco plants transformed with the herein disclosed nucleic acid encoding the protein set forth in SEQ ID NO: 1, had a significantly increased fruit weight, as compared to wild-type or GFP transformed tobacco plants.

### Example 4—Arabidopsis Plants with Increased Shoot Fresh Weight

In order to evaluate the effect of transformation in additional plant species, Arabidopsis thaliana, variant Columbia 4, was transformed with the herein disclosed nucleic acid set forth in SEQ ID NO: 4 to express the protein having the sequence set forth in SEQ ID NO: 1. Three independent events of T3 generation were evaluated under normal growth conditions in a temperature controlled green house with regular irrigation and fertilization regimes. Before beginning of senescence, fresh green organs including rosette leaves and total reproductive organs (stem, cauline leaves and siliques with seeds) were dissected and weighed.

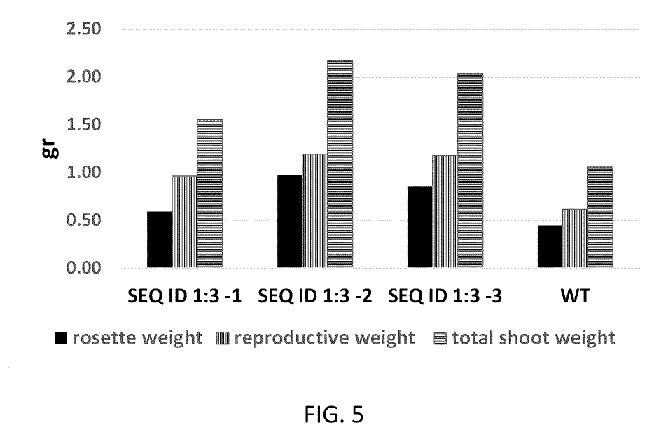

FIG. 5 shows that the rosette weight (in black), the reproductive weight (vertical strips), and the total shoot fresh weight (horizontal stripes) of the transformed plants increased significantly in comparison to the wild type plants, indicating that the stay-green phenotype is a type A or type B phenotype, increasing agricultural yield.

### Example 5—Tobacco Plants with Increased Agricultural Productivity

Tobacco plants (Nicotiana tabacum, cv. Little Dutch) were transformed with the nucleic acid set forth in SEQ ID NO: 5 encoding the amino acid set forth in SEQ ID NO: 2. Five single copy gene insert independent events were verified for homozygosity and selected for further experiments.

The plants were grown in the field under a net and drip irrigated and fertilized as needed for 16 weeks.

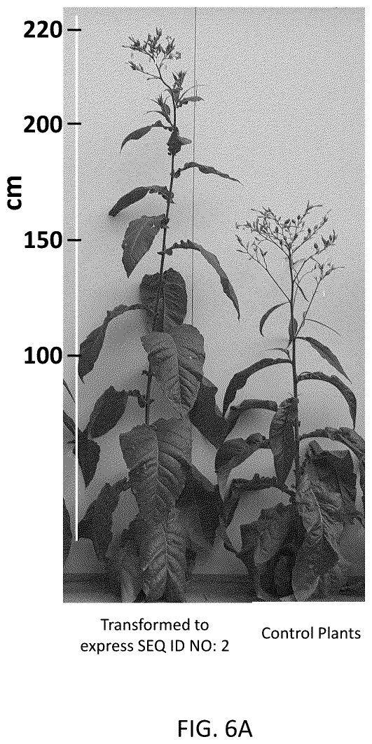

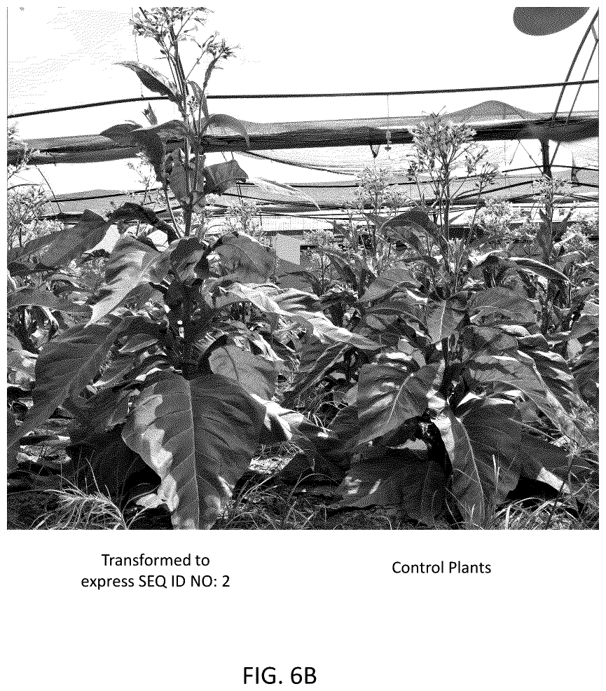

After 10 weeks, the plants were visually inspected. After 14 weeks, the shoots/fruits were collected and weighted. As seen from FIG. 6A and FIG. 6B, which shows pictures from tobacco plants grown in pots and in field, respectively, different transformation event of tobacco plants, were significantly larger compared to wild type plants, i.e. have a significantly larger agricultural output, since the leaves of the plants are the produce.

### Example 6—Tobacco Plant with Increased Height and Leaf Area

The increased size of the transformed tobacco plant was further evaluated by measuring the weight and height of the transformed tobacco plants as compared to wild type. The plants were grown under normal growth conditions as in example 3 and then the height and leaf area of the plants were measured.

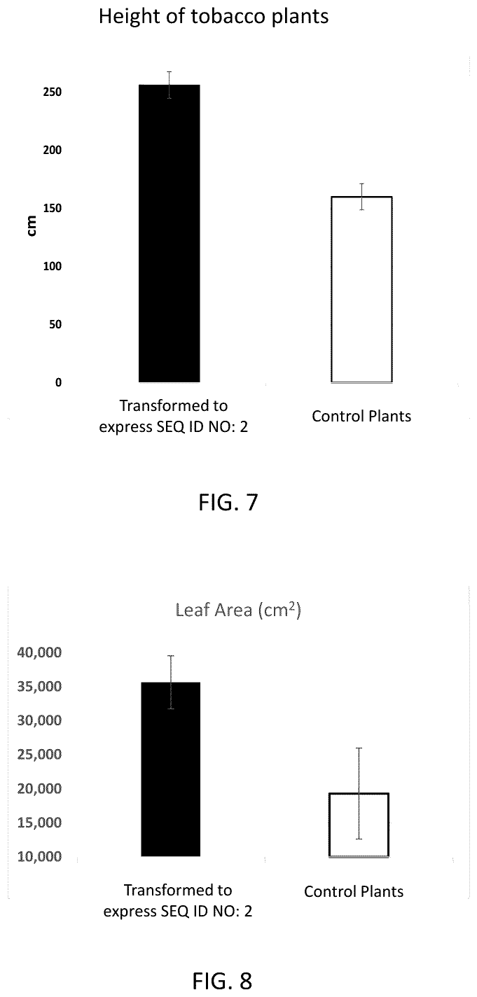

FIG. 7 shows that the tobacco plants transformed with the nucleic acid sequence set forth in SEQ ID NO: 5 the herein disclosed nucleic acid encoding the protein set forth in SEQ ID NO: 2 were significantly higher than wild type plants.

In addition, as seen from FIG. 8, the tobacco plants transformed with the herein disclosed nucleic acid encoding the protein set forth in SEQ ID NO: 2, had a significantly increased leaf area, as compared to wild-type tobacco plants.

### Example 7—Tobacco Plants with Increased Shoot Fresh Weight

In order to further validate the increased agricultural productivity of the tobacco plants transformed with the nucleic acid set forth in SEQ ID NO: 5 shoot fresh weight was measured.

The plants were grown under normal growth conditions for 16 weeks (as in example 5) the shoots were collected and weighted.

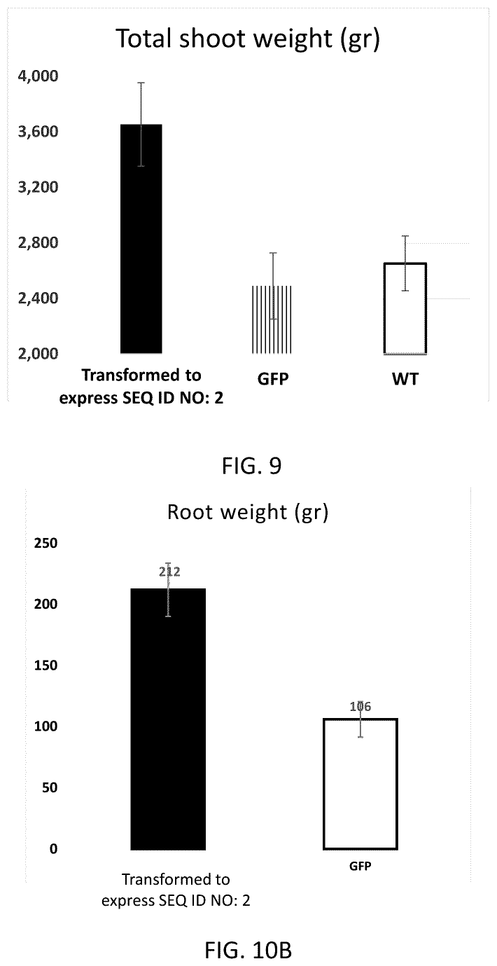

As seen from FIG. 9, the tobacco plants transformed with the herein disclosed nucleic acid encoding the protein set forth in SEQ ID NO: 2, had a significantly increased shoot weight, as compared with plants transformed with GFP or wild type plants as controls.

This clearly indicates the ability of polypeptide having the amino acid sequence set forth in SEQ ID NO: 2 to increase the agricultural productivity of the tobacco plant.

### Example 8—Tobacco Plants with Increased Root Weight

The phenotype of the transformed tobacco plant was further evaluated by measuring the volume and weight of the roots of the transformed tobacco plant in comparison to GFP expressing plants.

The plants were grown for 12 weeks (as in example 3), the plants were harvested, and the root size and weight were measured.

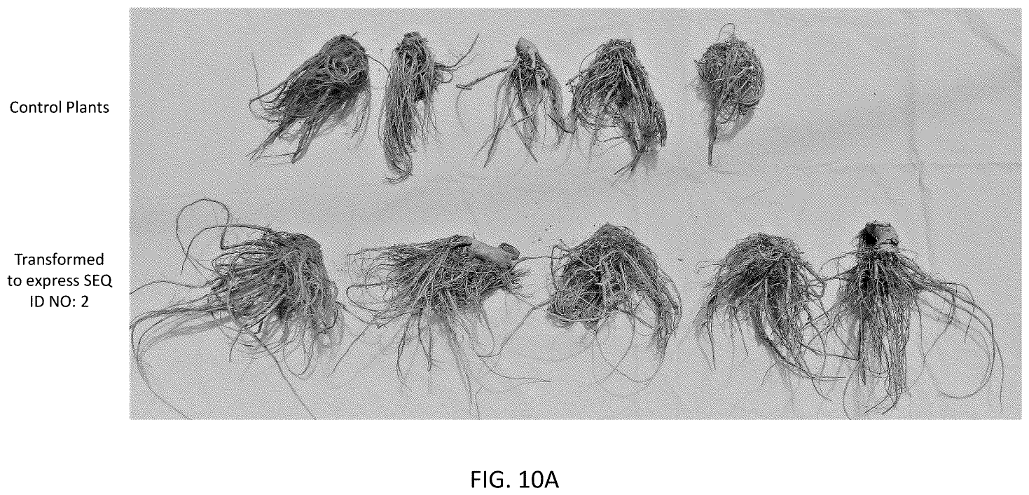

As seen from FIG. 10A and FIG. 10B, the tobacco plants transformed with the nucleic acid sequence set forth in SEQ ID NO: 5 encoding the protein set forth in SEQ ID NO: 2, had a significantly larger and more branched root as compared to the GFP expressing plants resulting in an overall increase in root weight.

Such increase in plant root size and weight is advantageous, since the root system is the major organ responsible for water and nutrient absorption, and the location where physiologically active substances, such as certain amino acid and hormones are synthesized. It is known that morphology and physiological characteristics of roots, collectively referred to as root volume, improve the nutrient absorption and increase the aboveground and belowground biomass.

### Example 9—Arabidopsis Plants with Increased Shoot Fresh Weight

In order to evaluate the effect of transformation with SEQ ID NO: 5 in additional plant species, Arabidopsis thaliana, variant Columbia 4, was transformed with SEQ ID NO: 5, T3 generation events were evaluated under normal growth conditions in a temperature controlled green house with regular irrigation and fertilization regimes, compared to wt plants.

Three independent events of Arabidopsis plants transformed with the nucleic acid set forth in SEQ ID NO: 5 to express the protein having the sequence set forth in SEQ ID NO: 2 and wt control plants were grown in the green house for 4 weeks. Before beginning of senescence, fresh green organs were dissected for rosette leaves and total reproductive organs (stem, cauline leaves, and siliques with seeds) and weighed.

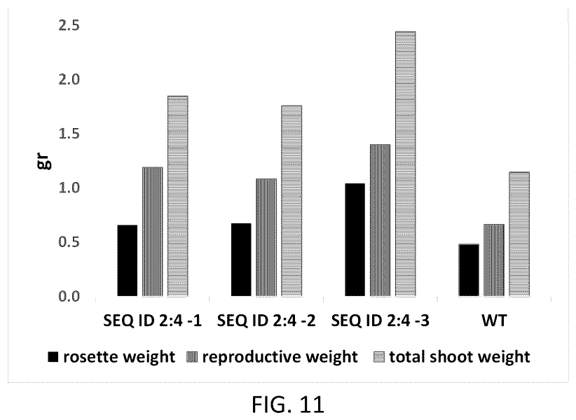

As seen from FIG. 11, the rosette weight (in black), the reproductive weight (vertical strips), and the total shoot fresh weight (horizontal stripes) of the transformed plants increased significantly in comparison to the wild type plant, indicating that the stay-green phenotype is a type A or type B phenotype, increasing agricultural yield.

### Example 10—Arabidopsis Plants Expressing Gene Variants of RanGAP1 Derived from a Fungus or from a Potato Plant

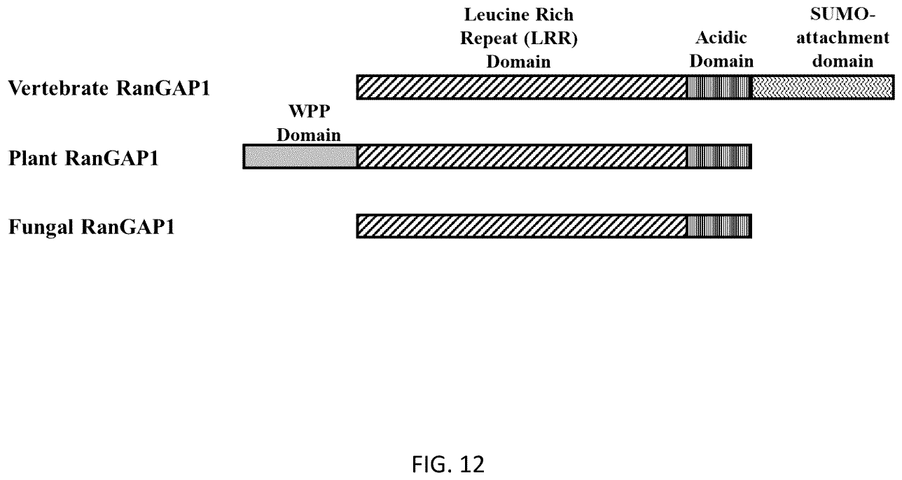

The RanGAP1 protein of vertebrates, plants, and fungi has a common Leucine-Rich Repeat (LRR) and Acidic Domains which are conserved across three kingdoms, while only plants have the additional and unique WPP domain (FIG. 12). The necessity of these domains and the advantage they may confer to Arabidopsis plant biomass was assessed, using naturally isolated (fungal-derived variants) and genetically changed (plant-derived variants) gene variants, and is disclosed hereinbelow.

Methods:

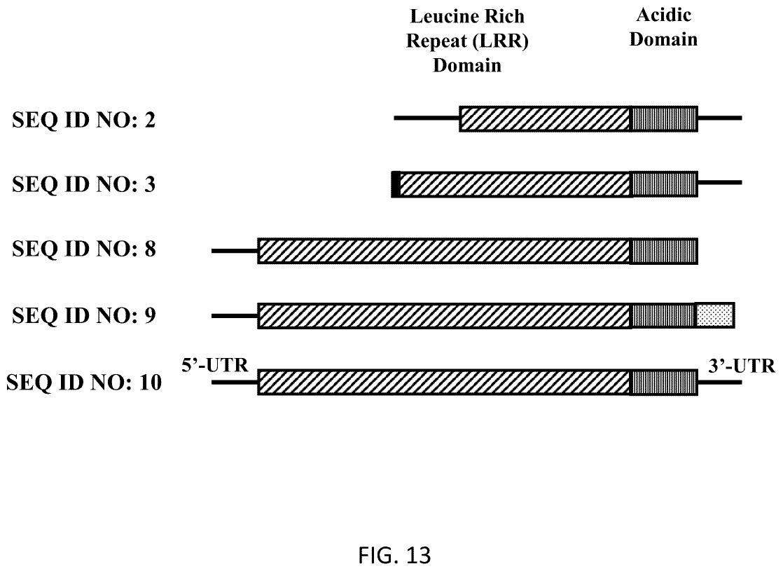

Cloning of fungal RanGAP1 variants—Fungal RanGAP1 gene's transcript variants were amplified from the cDNA derived from environmental RNA samples by PCR with specific primers targeting the 3′-UTR or the coding sequence 5′-end of the fungal RanGAP1 gene. The amplified DNA was cloned to binary vectors and transformed into bacterial host cells. Variants of the RanGAP1 fungal genes were screened by colony PCR performed on the transformed cells. Several variants were characterized by sanger sequencing as having a complete coding sequence with or without UTR regions at their 3′- or 5′-ends (SEQ ID NO: 5 and SEQ ID NO: 20-22 encoding the amino acid sequence of SEQ ID NO 2 and SEQ ID NO: 8-10, respectively) (FIG. 13). SEQ ID NO: 5 was found to be a partial gene lacking its 5′-end and most of the coding sequence and its translated amino acid sequence is SEQ ID NO: 2. To obtain the amino acid sequence of SEQ ID NO: 3, site-directed mutagenesis was performed on the binary vector containing the DNA sequence of SEQ ID NO: 5 to replace the Glycine coding triad to a Methionine (ATG START codon) at its 5′-end. Schematic illustration of the transcripts of the fungal RanGAP1 variants cloned to the binary vector is presented in FIG. 13.

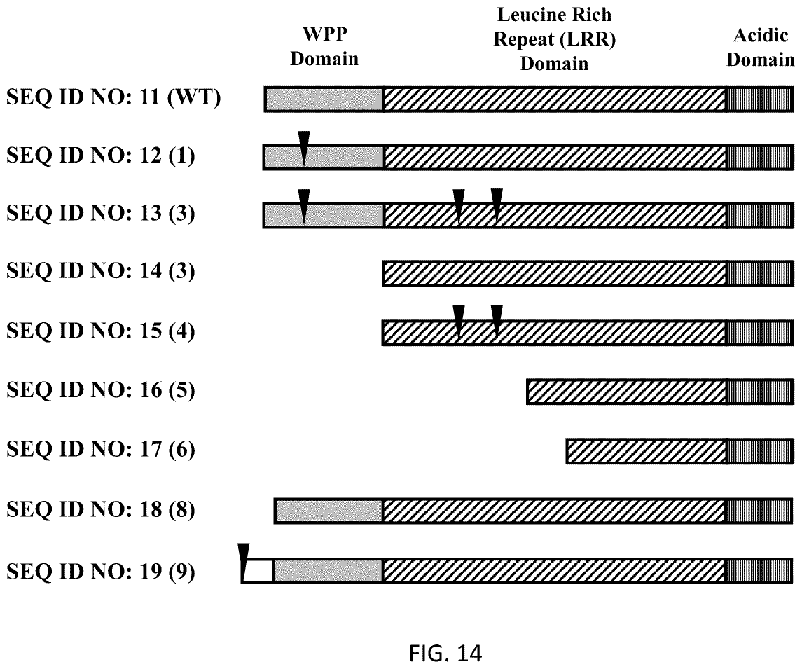

Cloning of potato RanGAP1 protein variants—The native RanGAP1 gene of potato (Solanum tuberosum) was amplified and cloned to a binary vector. The cloned DNA sequence of the potato RanGAP1 (StRanGAP1; SEQ ID NO: 23 encoding the amino acid sequence of SEQ ID NO: 11) was subjected to modifications yielding SEQ ID NO: 24-31 encoding the amino acid sequence of SEQ ID NO: 12-19. Schematic illustration of the translated regions of the potato RanGAP1 protein variants cloned to the binary vector is presented in FIG. 14

Experimental design—To evaluate the effect of the expression of RanGap1 gene variants, according to the amino acid sequences set forth in SEQ ID NO: 2, 8-17, Arabidopsis thaliana col-4 plants were transformed with nucleic acids corresponding to the sequences set forth in SEQ ID NO: 5, 20-29.

T3 generation events were evaluated under normal growth conditions in a temperature controlled green house with regular irrigation and fertilization regimes, compared to wild type (WT) plants in three independent trials.

In the first trial (#1), eight independent events of Arabidopsis plants transformed with the nucleic acid set forth in SEQ ID NO: 5, 20-22, and 28-29 to express the protein having the sequence set forth in SEQ ID NO: 2, 8-10 and 16-17 and fifteen WT control plants were grown in the greenhouse for 5 weeks. As flowers developed, fresh shoots (without roots) were dissected and weighed.

In the second trial (#2), seven independent events of Arabidopsis plants transformed with the nucleic acid set forth in SEQ ID NO: 5, 20, and 23-29 to express the protein having the sequence set forth in SEQ ID NO: 2, 8 and 11-17 and forty WT control plants were grown in the greenhouse for 3.5 weeks. Before the beginning of senescence, fresh green organs were dissected and weighed.

In the third trial (#3), seven independent events of Arabidopsis plants transformed with the nucleic acid set forth in SEQ ID NO: 20 and 23-29 to express the protein having the sequence set forth in SEQ ID NO: 8 and 11-17 and forty WT control plants were grown in the greenhouse for 3.5 weeks. As flowers developed, fresh shoots (without roots) were dissected and weighed.

Analysis scoring method—Each event was evaluated based on average total weight and a score was issued for each trial proceeding an evaluation in all three trials. In trials #1, #2, and #3, the events were sorted based on average total weight followed by scoring each event with a number between 1-4 according to the following division criteria:

In trials #1 and #3, a score of 1, for top ten performing events; a score of 2, for events above the median; a score of 3, for events above the WT average weight; and a score of 4, for events below the WT average weight.

In trial #2, a score of 1, for top ten performing events; a score of 2, for events above the WT average weight; a score of 3, for events above the median; and a score of 4, for events below the median.

Median and total average weight gave similar values in all trials (2.57 and 2.57 in trial #1, 2.56 and 2.66 in trial #2 and 2.77 and 2.74 in trial #3 respectively). Thus, the median was selected as a cutoff value.

All events were aligned and scores from the three trials were sorted by value to produce a string of all scores. Each string provided a score based on a scoring array. The top 3-4 performing events of each gene were then sorted into four groups as previously done: a score of 1, for top ten performing events; a score of 2, for events above the median; a score of 3, for events above the control score; and a score of 4, for events below the control score. The control score was set at the point between scores of 3 and a single score of 4 representing total weight below the control average weight (array 33-334).

Results:

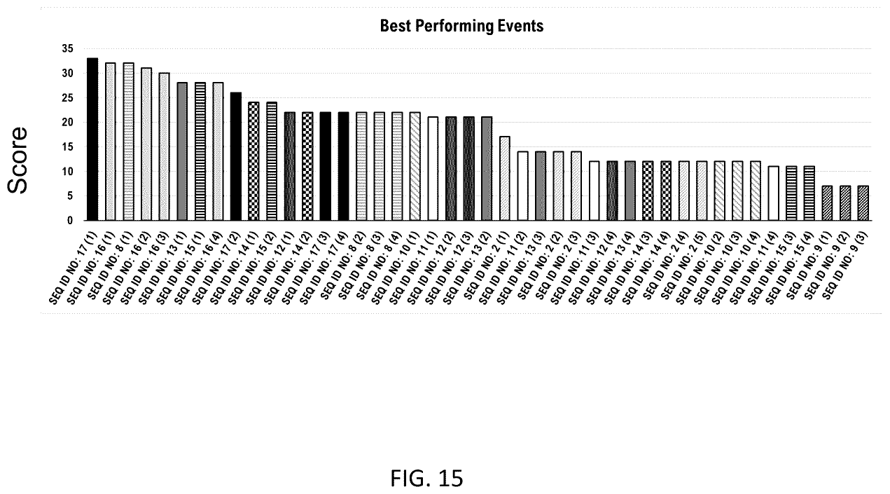

Effect of various fungal-derived RanGap1 gene variants on Arabidopsis biomass—Arabidopsis plants were transformed with the herein disclosed nucleic acid encoding the fungal derived variants of RanGap1 proteins whose amino acid sequence is set forth in SEQ ID NO: 2 and 8-10. As indicated by the results presented in FIG. 15, plants transformed with the nucleic acid encoding the variants of the RanGap1 proteins having amino acid sequences as set forth in SEQ ID NO: 2, 8, and 10 had increased weight, as compared with wild type plants, as indicated by the average total weight that includes fresh shoots (without roots) weight as measured as flowers developed, and fresh green organs weight as measured before the beginning of senescence.

The full-length fungal RanGap1 protein encoded by SEQ ID NO: 8 had significantly increased shoot weight compared to the partial gene variant encoded by SEQ ID NO: 2. The full-length fungal RanGap1 protein encoded by SEQ ID NO: 8 had significantly increased shoot weight also compared to transform plants over-expressing the potato plant native RanGAP1 encoded by SEQ ID NO: 11.

This clearly indicates the advantageous ability of fungal RanGap1 gene variants and specifically the ability of fungal RanGap1 proteins having amino acid sequences as set forth in SEQ ID NO: 8 and SEQ ID NO: 10 to increase the total weight productivity of the Arabidopsis plant compared to wild-type plants.

Effect of various potato-derived RanGap1 gene variants on Arabidopsis biomass—Arabidopsis plants were transformed with the herein disclosed nucleic acid encoding the potato-derived native RanGap1 protein and variants thereof, whose amino acid sequence is set forth in SEQ ID NO: 11-17. As indicated by the results presented in FIG. 15, plants transformed with the potato derived native RanGap1 protein and variants thereof had increased weight, as compared with wild type plants, as indicated by the average total weight that includes fresh shoots (without roots) weight as measured as flowers developed, and fresh green organs weight as measured before the beginning of senescence.

Potato-derived RanGap1 gene variants having amino acid sequences as set forth in SEQ ID NO: 16-17 had significantly increased shoot weight compared to the native protein SEQ ID NO: 11.

This clearly indicates the advantageous and surprising ability of potato-derived RanGap1 gene variants, and specifically, the ability of the shorter potato-derived RanGap1 proteins having amino acid sequences as set forth in SEQ ID NO: 14-17, to increase the total weight productivity of the Arabidopsis plant vs. wild-type plants and plants transformed with native RanGAP1. This result was unexpected and surprising considering that these variants lack substantial parts of the WPP domain which is unique to plants RanGap1 proteins

Moreover, equally surprising is the disclosed result that the highest number of observed events of increase in total weight productivity (FIG. 15) was of plants transformed to express the shortest and most partial, potato-derived RanGap1 protein variants (FIG. 14) having the amino acid sequences set forth in SEQ ID NO: 16 and SEQ ID NO: 17. This result was even more unexpected and surprising considering that these variants lack substantial parts of the WPP domain which is unique to plants RanGap1 proteins, and further lack parts of the Leucine-Rich Repeat (LRR) domain which is common and conserved across kingdoms (FIG. 12).

### Example 11—Arabidopsis Plants Expressing Gene Variants of RanGAP1 Derived from Various Plants

Cloning of potato RanGAP1 protein variants: The native RanGAP1 gene of potato (Solanum tuberosum; StRanGAP1), soy (Glycine max; GmRanGAP1), Arabidopsis (Arabidopsis thahana; AtRanGAP1) and rice (Oryza sativa; OsRanGAP1) were amplified and cloned to a binary vector under the control of a 35S constitutive promoter. The AtRanGAP1 was synthesized with modified codon usage to prevent co-suppression. The cloned DNA sequence of the plant RanGAP1s were modified yielding eight variant types:
Type (0) are the native full-length proteins. Potato RanGAP1 (StRanGAP1; SEQ ID NO: 23 encoding the amino acid sequence of SEQ ID NO: 11), soy RanGAP1 (GmRanGAP1; SEQ ID NO: 64 encoding the amino acid sequence of SEQ ID NO: 58), rice RanGAP1 (OsRanGAP1; SEQ ID NO: 76 encoding the amino acid sequence of SEQ ID NO: 70) and Arabidopsis RanGAP1 (AtRanGAP1; SEQ ID NO: 108 encoding the amino acid sequence of SEQ ID NO: 38);
Type (1): potato RanGAP1 type1 (St_1; SEQ ID NO: 24 encoding the amino acid sequence of SEQ ID NO: 12);
Type (2): potato RanGAP1 type2 (St_2; SEQ ID NO: 25 encoding the amino acid sequence of SEQ ID NO: 13);
Type (3): potato RanGAP1 type3 (St_3; SEQ ID NO: 26 encoding the amino acid sequence of SEQ ID NO: 14), soy RanGAP1 type3 (Gm 3; SEQ ID NO: 65 encoding the amino acid sequence of SEQ ID NO: 59) and Arabidopsis RanGAP1 type3 (At_3; SEQ ID NO: 109 encoding the amino acid sequence of SEQ ID NO: 39);
Type (4): potato RanGAP1 type4 (St_4; SEQ ID NO: 27 encoding the amino acid sequence of SEQ ID NO: 15);
Type (5): potato RanGAP1 type5 (St_5; SEQ ID NO: 28 encoding the amino acid sequence of SEQ ID NO: 16), rice RanGAP1 type5 (Os_5; SEQ ID NO: 78 encoding the amino acid sequence of SEQ ID NO: 72) and Arabidopsis RanGAP1 type5 (At_5; SEQ ID NO: 110 encoding the amino acid sequence of SEQ ID NO: 40);
Type (6): potato RanGAP1 type6 (St_6; SEQ ID NO: 29 encoding the amino acid sequence of SEQ ID NO: 17), soy RanGAP1 type6 (Gm 6; SEQ ID NO: 67 encoding the amino acid sequence of SEQ ID NO: 61) and Arabidopsis RanGAP1 type6 (At_6; SEQ ID NO: 111 encoding the amino acid sequence of SEQ ID NO: 41);
Type (8): potato RanGAP1 type8 (St_8; SEQ ID NO: 30 encoding the amino acid sequence of SEQ ID NO: 18), soy RanGAP1 type8 (Gm_8; SEQ ID NO: 68 encoding the amino acid sequence of SEQ ID NO: 62) and Arabidopsis RanGAP1 type8 (At_8; SEQ ID NO: 112 encoding the amino acid sequence of SEQ ID NO: 42);
Type (9): potato RanGAP1 type9 (St_9; SEQ ID NO: 31 encoding the amino acid sequence of SEQ ID NO: 19), soy RanGAP1 type9 (Gm_8; SEQ ID NO: 69 encoding the amino acid sequence of SEQ ID NO: 63) and rice RanGAP1 type9 (Os_9; SEQ ID NO: 81 encoding the amino acid sequence of SEQ ID NO: 75).

Schematic illustration of the translated regions of the potato RanGAP1 protein variants corresponding to all plant RanGAP1 proteins cloned to the binary vector is presented in FIG. 14.

Experimental design—To evaluate the effect of expression of RanGap1 gene variants on phenotype of plants, Arabidopsis thaliana cv. col4 plants were transformed with the herein disclosed nucleic acid encoding the RanGap1 protein and variants thereof from a variety of plants including potato (SEQ ID NO: 16-18); soy (SEQ ID NO: 58, 59 and 61-63); rice (SEQ ID NO: 70, 72 and 75); and Arabidopsis (SEQ ID NO: 39-42).

Events of genetically transformed Arabidopsis Thaliana (col4) lines were created by Agrobacterium mediated transformation. Plants were germinated on peat, moss and coco-peat mixture, and treated with Basta herbicide (Bayer, active ingredient glufosinate-ammonium), to select positive transgenic plants. Transformed plants (events) of each vector were transplanted to a 1.5-liter pots each. Wild-type plants and empty vector lines (transformed with the same binary vector, with Bialaphos resistance, without the addition of RanGap1 gene) were used.

Plants were grown for 4 weeks until bolting. T1 generation events expressing RanGap1 gene variants, according to the amino acid sequences set forth in SEQ ID NO: 39-42, 58, 59, 61-63 and 70, 72 and 75 and selected T2 events of RanGap1 gene variants having the amino acid sequences set forth in SEQ ID NO: 16-18 were evaluated under normal growth conditions in a temperature controlled green house with regular irrigation and fertilization regimes. 4 independent events T1 of transformed Arabidopsis plants, 10 T2 plants from each event, 7 events of Arabidopsis plants transformed with empty vector and 7 WT control plants were evaluated.

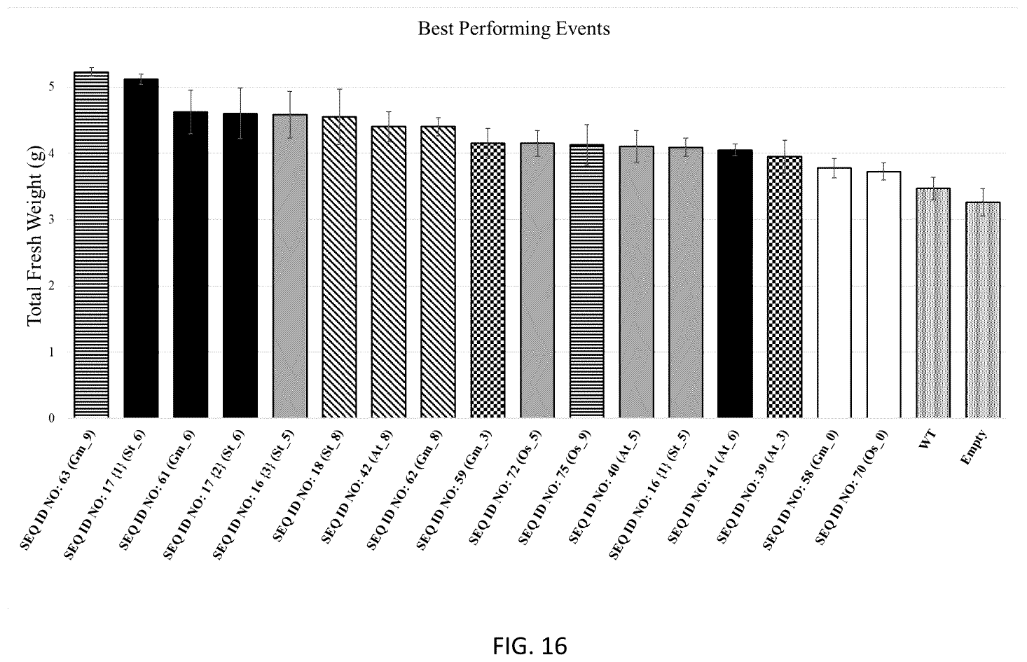

As seen from FIG. 16, all plants transformed with the plant derived native RanGap1 protein and variants thereof had increased weight, as compared with wild type plants and empty vector transformed plants, as indicated by the average total weigh and ±Standard Error of the different constructs.

Surprisingly, expression of RanGap1 variants further increased the obtained biomass. Excelling constructs were:
Two type9 variants, namely one having SEQ ID NO: 63 (soy) and one having SEQ ID NO: 75 (rice);
Three type 6 variants, namely two having SEQ ID NO: 17 (potato), and one having SEQ ID NO: 61 (soy);
Four type5 variants, namely two having SEQ ID NO: 16 (potato), one having SEQ ID NO: 72 (rice), and one having SEQ ID NO: 40 (Arabidopsis); and
Three type8 variants (encoding same protein as type9 but with different DNA sequence), namely one having of SEQ ID NO: 18 (potato), one having of SEQ ID NO: 42 (Arabidopsis) and one having of SEQ ID NO: 62 (soy).

These results clearly show the advantageous ability of plants expressing variants of the RanGap1 gene, in particular RanGap1 variant types 5, 6, 8 and 9, that do not contain the WPP motif, in increasing the total weight productivity of the plant, as compared to plants that do not contain these expressed sequences or that express RanGap1 with a WPP domain.

The experiment is further repeated utilizing the RanGAP1 variants of Alfalfa (Medicago sativa), barley (Hordeum vulgare), carrot (Daucus carota), cassava (Manihot esculenta), clover (Trifolium subterraneum), cocoa (Theobroma cacao), coffee (Coffea arabica), Corn (Zea mays), Cotton (Gossypium hirsutum), cowpea (Vigna unguiculata), Eucalyptus sp., Pinus spp., lettuce (Lactuca sativa), peanut (Arachis hypogaea), Rape seed (Canola) (Brassica napus), Sorghum (Sorghum bicolor), Sugar beet (Beta vulgaris), switchgrass (Panicum virgatum), Tobacco (Nicotania tabacum), tomato (Solanum lycopersicum), and/or wheat (Triticum aestivum).

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1-34. (canceled)

35. A plant genetically modified to express a nucleic acid sequence encoding a RanGAP1 protein having a mutated, truncated or deleted WPP motif and/or domain.

36. The plant of claim 35, further having a mutated, truncated or deleted Leucine-Rich Repeat (LRR) domain.

37. The plant of claim 35, wherein the amino acid sequence of the genetically modified RanGAP1 has at least 90% sequence homology to any one of SEQ ID NO: 12-19, 32-34, 39-42, 49-52, 59-63, 71-75.

38. The plant of claim 35, wherein the nucleotide sequence of the genetically modified RanGAP1 has at least 90% sequence homology to any one of SEQ ID NO: 24-31 35-37, 44-47, 54-57, 65-69, 77-81 or 88.

39. The plant of claim 35, wherein the amino acid sequence of the genetically modified RanGAP1 has at least 90% sequence homology to any one of SEQ ID NO: 3, 8-10 or 82-84.

40. The plant of claim 35, wherein the plant has an increased agricultural productivity when grown under normal growth conditions, as compared to its wild-type counterpart; and wherein increasing the agricultural productivity of the plant comprises one or more of increasing a leaf area of the plant, increasing fresh weight of the plant, increasing fresh green organ weight, increasing shoot fresh weight (with and/or without roots), increasing fruit weight, increasing plant height, increasing root weight and volume, increasing the growth rate of the plant, increased seed weight of the plant, increased dry weight of plant organs.

41. The plant of claim 35, wherein the RanGAP1 protein is an ortholog RanGAP1 protein derived from a plant.

42. The plant according to claim 41, wherein the RanGAP1 ortholog is derived from potato (Solanum tuberosum), Arabidopsis thaliana, Camelina sativa, soy (Glycine Max), rice (Oryza sativa), Alfalfa (Medicago sativa), barley (Hordeum vulgare), carrot (Daucus carota), cassava (Manihot esculenta), clover (Trifolium subterraneum), cocoa (Theobroma cacao), coffee (Coffea arabica), Corn (Zea mays), Eucalyptus sp., Pinus spp., Cotton (Gossypium hirsutum), cowpea (Vigna unguiculata), lettuce (Lactuca sativa), peanut (Arachis hypogaea), Rape seed (Canola) (Brassica napus), Sorghum (Sorghum bicolor), Sugar beet (Beta vulgaris), switchgrass (Panicum virgatum), Tobacco (Nicotania tabacum), tomato (Solanum lycopersicum), or wheat (Triticum aestivum).

43. The plant of claim 35, wherein the RanGAP1 protein is a gene edited version of the endogenous RanGAP1 protein of the plant.

44. The plant of claim 35, further expressing a nucleic acid sequence encoding a protein having at least 90% sequence homology to a putative non-plant SRF-TF-domain-containing protein having 90% sequence homology to the amino acid sequence set forth in SEQ ID NO: 1.

45. A method for increasing agricultural productivity of a plant, the method comprising genetically modifying the plant to express a nucleic acid sequence encoding a RanGAP1 protein having a mutated, truncated or deleted WPP motif and/or domain, thereby increasing the agricultural productivity of the plant when grown under normal growth conditions, as compared to wild type plants.

46. The method of claim 45, wherein the genetically modifying comprises transforming the plant with an exogenous nucleic acid sequence encoding an ortholog RanGAP1 protein having a mutated, truncated or deleted WPP motif and/or domain.

47. The method according to claim 45, wherein the RanGAP1 ortholog is derived from potato (Solanum tuberosum), Arabidopsis thaliana, Camelina sativa, soy (Glycine Max), rice (Oryza sativa), Alfalfa (Medicago sativa), barley (Hordeum vulgare), carrot (Daucus carota), cassava (Manihot esculenta), clover (Trifolium subterraneum), cocoa (Theobroma cacao), coffee (Coffea arabica), Corn (Zea mays), Eucalyptus sp., Pinus spp., Cotton (Gossypium hirsutum), cowpea (Vigna unguiculata), lettuce (Lactuca sativa), peanut (Arachis hypogaea), Rape seed (Canola) (Brassica napus), Sorghum (Sorghum bicolor), Sugar beet (Beta vulgaris), switchgrass (Panicum virgatum), Tobacco (Nicotania tabacum), tomato (Solanum lycopersicum), or wheat (Triticum aestivum).

48. The method of claim 45, wherein the genetically modifying comprises gene editing the nucleotide sequence of the RanGAP1 protein endogenous to the plant to obtain a RanGAP1 protein having a mutated, truncated or deleted WPP motif and/or domain.

49. The method of claim 45, wherein the amino acid sequence of the genetically modified RanGAP1 has at least 90% sequence homology to any one of SEQ ID NO: 12-19, 32-34, 39-42, 49-52, 59-63, 71-75.

50. The method of claim 45, wherein the nucleotide sequence of the genetically modified RanGAP1 has at least 90% sequence homology to any one of SEQ ID NO: 24-31 35-37, 44-47, 54-57, 65-69, 77-81 or 88.

51. The method of claim 45, wherein the amino acid sequence of the genetically modified RanGAP1 has at least 90% sequence homology to any one of SEQ ID NO:14, 16, 17, 18, 19, 39, 49, 59, 71, 40, 50, 60, 72, 41, 51, 61, 73, 42, 52, 62, 74, 63 or 75.

52. The method of claim 45, wherein the amino acid sequence of the genetically modified RanGAP1 has at least 90% sequence homology to any one of SEQ ID NO: 3, 8-10 or 82-84.

53. The method of claim 45, wherein the genetically modifying further comprises expressing a nucleic acid sequence encoding a protein having at least 70% sequence homology to a putative SRF-TF-domain-containing protein ortholog having at least 90% sequence homology to the amino acid sequence set forth in SEQ ID NO: 1.

54. A vector comprising a nucleic acid encoding a protein having at least 90% sequence homology to RanGAP1 protein having a mutated, truncated or deleted WPP domain.


SEQUENCE LISTING (112 sequences)

SEQ ID NO: 1 - protein, 221 aa, unidentified
  REGION 1..221: note = metagenome purified from environmental niche
MADITAVGEENPSPTQDELQQAAAGNGAPDNRTPKRRMSDDEEDEEKQGRERRKIEIKFIQDKSRRHITFSKRKAGIMKKAYELSVLTGTQVLLLVVSETGLVYTFTTPKLQPLVTKAEGKNLIQACLNAPDPTTSENGVDAPEVPAETPEDVNHANVNAAAAQQTNIPRPTGMHPGYMTNEQQQQMAYYQNHLQQQQQAGGQYPGMSVGGRMPTQHQPTA

SEQ ID NO: 2 - protein, 213 aa, unidentified
  REGION 1..213: note = metagenome purified from environmental niche
MPQNGIRMEGIKAIADGLAKCPTLEVLDLQDNTATKTGTRSIVRHLSTWPKLRILNLSDCLLGSVGGIALATALSTGSNKHLEQLKLQYGEFDKRTVEILSTAISQHLPKLTTLELNGNRFDAEDECVETLKKALELHGNEDALDELDDMEEVDEDEEDDDDEDEEDEDEDKDTSADDGIDAGAAGEDALPPVTKKDEDVLADLLSKVHVQPS

SEQ ID NO: 3 - protein, 271 aa, unidentified
  REGION 1..271: note = metagenome purified from environmental niche
MGGTVVSNALLENAKLCKTQGKESSLRVIVCGRNRLENGSAPHWAEAFATHGKLVEVRMPQNGIRMEGIKAIADGLAKCPTLEVLDLQDNTATKTGTRSIVRHLSTWPKLRILNLSDCLLGSVGGIALATALSTGSNKHLEQLKLQYGEFDKRTVEILSTAISQHLPKLTTLELNGNRFDAEDECVETLKKALELHGNEDALDELDDMEEVDEDEEDDDDEDEEDEDEDKDTSADDGIDAGAAGEDALPPVTKKDEDVLADLLSKVHVQPS

SEQ ID NO: 4 - DNA, 666 bp, unidentified
  misc_feature 1..666: note = metagenome purified from environmental niche
  source 1..666: mol_type = unassigned DNA
atggccgacatcactgccgtcggtgaggagaacccttctcctacccaggatgagctgcagcaggccgcggccggtaacggcgctcctgataaccgcactcccaagcgtcgcatgagtgacgatgaagaggacgaggagaagcagggtcgcgagcgcagaaagattgagatcaagttcattcaggataagtcgcgtcgccacatcaccttctccaagcggaaggcgggtatcatgaagaaggcatacgaattgtccgtcctcacaggcacccaggtgctgttgctggtcgtgtccgagaccggcctggtctatacctttaccacccctaagctccaaccattggtcaccaaggcggagggcaagaacctgattcaggcttgcctcaacgcccccgaccctaccaccagcgagaatggcgtcgatgcccccgaggtcccagcggagacccccgaggatgtcaaccacgccaacgtcaacgctgccgcagcccagcagaccaacatccctcgtcccaccggaatgcatcccggctacatgaccaacgaacaacagcagcagatggcctactaccaaaaccacctccagcagcaacagcaggccggtgggcagtaccctggcatgtctgtcggtggtcgcatgcctacgcagcaccagcctaccgcataa

SEQ ID NO: 5 - DNA, 816 bp, unidentified
  misc_feature 1..816: note = metagenome purified from environmental niche
  source 1..816: mol_type = unassigned DNA
gggggaggaacggtcgttagcaatgctttgctggagaatgccaagctctgcaagacccagggcaaggagagctctcttcgagtcatcgtgtgtggccgaaataggttggagaatgggtctgcacctcattgggccgaggcgtttgctacgcatggcaaattggtggaagtgaggatgccgcaaaacggcattcgcatggagggcatcaaagctatcgccgacggactggccaagtgtccgacattggaagtgcttgatttgcaggacaacacggctaccaagacaggaacacggagtattgtccgacacctctcaacttggcctaaacttcgaatactcaatctctcggactgtcttttgggttcggtcggcggtatcgctcttgcaaccgcattgtccactggctcgaacaagcacctcgaacagctcaaactgcaatatggcgagtttgacaagaggacggttgagatactgtcgacggcaattagccagcatttgccaaaattgacgacactcgaactgaatggaaaccgtttcgatgccgaagacgaatgcgttgagaccctgaagaaggcacttgagctacatgggaacgaggatgctttggacgaacttgacgatatggaggaggtggacgaggacgaagaggatgatgatgacgaggacgaggaggacgaagacgaggacaaggacactagcgccgacgatgggatcgatgcaggagctgctggagaagacgctctaccaccagtcacgaagaaggacgaggacgtacttgcggatctcctgtccaaggtccacgttcagcctagctga

SEQ ID NO: 6 - DNA, 816 bp, unidentified
  misc_feature 1..816: note = metagenome purified from environmental niche
  source 1..816: mol_type = unassigned DNA
atgggaggaacggtcgttagcaatgctttgctggagaatgccaagctctgcaagacccagggcaaggagagctctcttcgagtcatcgtgtgtggccgaaataggttggagaatgggtctgcacctcattgggccgaggcgtttgctacgcatggcaaattggtggaagtgaggatgccgcaaaacggcattcgcatggagggcatcaaagctatcgccgacggactggccaagtgtccgacattggaagtgcttgatttgcaggacaacacggctaccaagacaggaacacggagtattgtccgacacctctcaacttggcctaaacttcgaatactcaatctctcggactgtcttttgggttcggtcggcggtatcgctcttgcaaccgcattgtccactggctcgaacaagcacctcgaacagctcaaactgcaatatggcgagtttgacaagaggacggttgagatactgtcgacggcaattagccagcatttgccaaaattgacgacactcgaactgaatggaaaccgtttcgatgccgaagacgaatgcgttgagaccctgaagaaggcacttgagctacatgggaacgaggatgctttggacgaacttgacgatatggaggaggtggacgaggacgaagaggatgatgatgacgaggacgaggaggacgaagacgaggacaaggacactagcgccgacgatgggatcgatgcaggagctgctggagaagacgctctaccaccagtcacgaagaaggacgaggacgtacttgcggatctcctgtccaaggtccacgttcagcctagctga

SEQ ID NO: 7 - DNA, 666 bp, unidentified
  misc_feature 1..666: note = metagenome purified from environmental niche
  source 1..666: mol_type = unassigned DNA
atggccgatattaccgcagttggcgaagagaatccctcccccactcaagacgagttgcagcaagcagccgccgggaatggagctccagataaccgtacacctaagcgtcgtatgagtgacgacgaagaggacgaggaaaaacagggaagggaaaggcgcaagattgagataaagtttatccaagacaagtctagaaggcatatcacattcagcaaaagaaaagctggcataatgaagaaggcatatgaactgagcgttctgaccggcacacaggttctccttttggtagtttccgagactggattggtgtatacatttactacccctaaactccagcctctggtaactaaagctgaagggaaaaatctcatacaagcctgtcttaacgcacccgatcccaccacatcagagaacggcgtcgacgctcccgaagtacccgccgagactcctgaggacgtgaaccacgcaaacgtcaatgcagctgctgcacagcagacaaacattccacgtccaactgggatgcacccaggctatatgactaacgaacagcaacaacagatggcctattaccagaatcatctgcaacagcagcagcaagcaggaggccaataccccggcatgagtgtaggagggaggatgcctacccaacatcaaccaaccgcttaa

SEQ ID NO: 8 - protein, 411 aa, unidentified
  REGION 1..411: note = metagenome purified from environmental niche
MSSSSSSKVFSILGKNLKANTAADLEPHLSELKDLNDVEEVHFGGNSLGVEACQAIAEVLKTKQSLKVVDLADVFTGRLISEIPQALSALCDALQSHTSLVELDLSDNAFGGRCADAMVPFLTNNTHFSVFKLNNNGLGIWGGTVVSNALLENAKLCKTQGKESSLRVIVCGRNRLENGSAPHWAEAFATHGKLVEVRMPQNGIRMEGIKAIADGLAKCPTLEVLDLQDNTATKTGTRSIVRHLSTWPKLRILNLSDCLLGSVGGIALATALSTGSNKHLEQLKLQYGEFDKRTVEILSTAISQHLPKLTTLELNGNRFDAEDECVETLKKALELHGNEDALDELDDMEEVDEDEEDDDDEDEEDEDEDKDTSADDGIDAGAAGEDALPPVTKKDEDELADLLSKVHVQPS

SEQ ID NO: 9 - protein, 418 aa, unidentified
  REGION 1..418: note = metagenome purified from environmental niche
MSSSSSSKVFSILGKNLKANTAADLEPHLSELKDLNDVEEVHFGGNSLGVEACQAIAEVLKTKQSLKVVDLADVFTGRLISEIPQALSALCDALQSHTSLVELDLSDNAFGGRCADAMVPFLTNNTHFSVFKLNNNGLGIWGGTVVSNALLENAKLCKTQGKESSLRVIVCGRNRLENGSAPHWAEAFATHGKLVEVRMPQNGIRMEGIKAIADGLAKCPTLEVLDLQDNTATKTGTRSIVRHLSTWPKLRILNLSDCLLGSVGGIALATALSTGSNKHLEQLKLQYGEFDKRTVEILSTAISQHLPKLTTLELNGNRFDAEDECVETLKKALELHGNEDALDELDDMEEVDEDEEDDDDEDEEDEDEDKDTSADDGIDAGAAGEDALPPVTKKDEDELADLLSKVHVQLAESHVRSL

SEQ ID NO: 10 - protein, 411 aa, unidentified; identical to SEQ ID NO: 8
  REGION 1..411: note = metagenome purified from environmental niche

SEQ ID NO: 11 - protein, 534 aa, Solanum tuberosum
MDSAGFSMKLWPPSLSTRLMLVERMTKNLITPSILSRKYGLLSKEEAEEDAKQIEALAFDSANQHFDKEPDGDGSSAVQLYAKESSKLMLEVIKRGPQTKESAEGIVSEKVKASDETTIFDISKGRRDFLSAEEASELLKPLSEPGNNYKRICFSNRSFGVDAAKIAGPILSSLKDQLTEVDLSDFVAGRPEEEALEVMEIFSSALDACDLRYLDLSNNALGEKGIRAFGALLKSQKNLEELYLMNDGISEEAAQAVCELIPSTDKLRILHFHNNMTGDEGALAISKLVKHSPALEDFRCSSTRVGSEGGVALSQALGECRNLKKVDLRDNMFGVEAGIALSKVLSIFSGLTEIYLSYLNLEDEGSIALANVLKESAPSLEVLEMDGNDITAKAAPALAACIAAKQFLTTLKLGENELKDEGAILIAKALEDGHGQLTEVDMSTNAIRRAGARCLAQAVVNKPGFKVLNINGNFISDEGIDEVKDIFKNSLHVLGPLDDNDPEGEDYDEEADEGGDNENDLETRLKDLDIKQEE

SEQ ID NO: 12 - protein, 534 aa, Solanum tuberosum
MDSAGFSMKLAPPSLSTRLMLVERMTKNLITPSILSRKYGLLSKEEAEEDAKQIEALAFDSANQHFDKEPDGDGSSAVQLYAKESSKLMLEVIKRGPQTKESAEGIVSEKVKASDETTIFDISKGRRDFLSAEEASELLKPLSEPGNNYKRICFSNRSFGVDAAKIAGPILSSLKDQLTEVDLSDFVAGRPEEEALEVMEIFSSALDACDLRYLDLSNNALGEKGIRAFGALLKSQKNLEELYLMNDGISEEAAQAVCELIPSTDKLRILHFHNNMTGDEGALAISKLVKHSPALEDFRCSSTRVGSEGGVALSQALGECRNLKKVDLRDNMFGVEAGIALSKVLSIFSGLTEIYLSYLNLEDEGSIALANVLKESAPSLEVLEMDGNDITAKAAPALAACIAAKQFLTTLKLGENELKDEGAILIAKALEDGHGQLTEVDMSTNAIRRAGARCLAQAVVNKPGFKVLNINGNFISDEGIDEVKDIFKNSLHVLGPLDDNDPEGEDYDEEADEGGDNENDLETRLKDLDIKQEE

SEQ ID NO: 13 - protein, 534 aa, Solanum tuberosum
MDSAGFSMKLAPPSLSTRLMLVERMTKNLITPSILSRKYGLLSKEEAEEDAKQIEALAFDSANQHFDKEPDGDGSSAVQLYAKESSKLMLEVIKRGPQTKESAEGIVSEKVKASDETTIFDISKGRRDFLSAEEASELLKPLSEPGNNYKRICFSNRSFGVDAAKIAGPILSSLKDQLTEVDLSDFVAGAPEEEALEVMEIFSSALDACDLRYLDLSNAALGEKGIRAFGALLKSQKNLEELYLMNDGISEEAAQAVCELIPSTDKLRILHFHNNMTGDEGALAISKLVKHSPALEDFRCSSTRVGSEGGVALSQALGECRNLKKVDLRDNMFGVEAGIALSKVLSIFSGLTEIYLSYLNLEDEGSIALANVLKESAPSLEVLEMDGNDITAKAAPALAACIAAKQFLTTLKLGENELKDEGAILIAKALEDGHGQLTEVDMSTNAIRRAGARCLAQAVVNKPGFKVLNINGNFISDEGIDEVKDIFKNSLHVLGPLDDNDPEGEDYDEEADEGGDNENDLETRLKDLDIKQEE

SEQ ID NO: 14 - protein, 413 aa, Solanum tuberosum
MSKGRRDFLSAEEASELLKPLSEPGNNYKRICFSNRSFGVDAAKIAGPILSSLKDQLTEVDLSDFVAGRPEEEALEVMEIFSSALDACDLRYLDLSNNALGEKGIRAFGALLKSQKNLEELYLMNDGISEEAAQAVCELIPSTDKLRILHFHNNMTGDEGALAISKLVKHSPALEDFRCSSTRVGSEGGVALSQALGECRNLKKVDLRDNMFGVEAGIALSKVLSIFSGLTEIYLSYLNLEDEGSIALANVLKESAPSLEVLEMDGNDITAKAAPALAACIAAKQFLTTLKLGENELKDEGAILIAKALEDGHGQLTEVDMSTNAIRRAGARCLAQAVVNKPGFKVLNINGNFISDEGIDEVKDIFKNSLHVLGPLDDNDPEGEDYDEEADEGGDNENDLETRLKDLDIKQEE

SEQ ID NO: 15 - protein, 413 aa, Solanum tuberosum
MSKGRRDFLSAEEASELLKPLSEPGNNYKRICFSNRSFGVDAAKIAGPILSSLKDQLTEVDLSDFVAGAPEEEALEVMEIFSSALDACDLRYLDLSNAALGEKGIRAFGALLKSQKNLEELYLMNDGISEEAAQAVCELIPSTDKLRILHFHNNMTGDEGALAISKLVKHSPALEDFRCSSTRVGSEGGVALSQALGECRNLKKVDLRDNMFGVEAGIALSKVLSIFSGLTEIYLSYLNLEDEGSIALANVLKESAPSLEVLEMDGNDITAKAAPALAACIAAKQFLTTLKLGENELKDEGAILIAKALEDGHGQLTEVDMSTNAIRRAGARCLAQAVVNKPGFKVLNINGNFISDEGIDEVKDIFKNSLHVLGPLDDNDPEGEDYDEEADEGGDNENDLETRLKDLDIKQEE

SEQ ID NO: 16 - protein, 241 aa, Solanum tuberosum
MLEDFRCSSTRVGSEGGVALSQALGECRNLKKVDLRDNMFGVEAGIALSKVLSIFSGLTEIYLSYLNLEDEGSIALANVLKESAPSLEVLEMDGNDITAKAAPALAACIAAKQFLTTLKLGENELKDEGAILIAKALEDGHGQLTEVDMSTNAIRRAGARCLAQAVVNKPGFKVLNINGNFISDEGIDEVKDIFKNSLHVLGPLDDNDPEGEDYDEEADEGGDNENDLETRLKDLDIKQEE

SEQ ID NO: 17 - protein, 228 aa, Solanum tuberosum
MEGGVALSQALGECRNLKKVDLRDNMFGVEAGIALSKVLSIFSGLTEIYLSYLNLEDEGSIALANVLKESAPSLEVLEMDGNDITAKAAPALAACIAAKQFLTTLKLGENELKDEGAILIAKALEDGHGQLTEVDMSTNAIRRAGARCLAQAVVNKPGFKVLNINGNFISDEGIDEVKDIFKNSLHVLGPLDDNDPEGEDYDEEADEGGDNENDLETRLKDLDIKQEE

SEQ ID NO: 18 - protein, 515 aa, Solanum tuberosum
MLVERMTKNLITPSILSRKYGLLSKEEAEEDAKQIEALAFDSANQHFDKEPDGDGSSAVQLYAKESSKLMLEVIKRGPQTKESAEGIVSEKVKASDETTIFDISKGRRDFLSAEEASELLKPLSEPGNNYKRICFSNRSFGVDAAKIAGPILSSLKDQLTEVDLSDFVAGRPEEEALEVMEIFSSALDACDLRYLDLSNNALGEKGIRAFGALLKSQKNLEELYLMNDGISEEAAQAVCELIPSTDKLRILHFHNNMTGDEGALAISKLVKHSPALEDFRCSSTRVGSEGGVALSQALGECRNLKKVDLRDNMFGVEAGIALSKVLSIFSGLTEIYLSYLNLEDEGSIALANVLKESAPSLEVLEMDGNDITAKAAPALAACIAAKQFLTTLKLGENELKDEGAILIAKALEDGHGQLTEVDMSTNAIRRAGARCLAQAVVNKPGFKVLNINGNFISDEGIDEVKDIFKNSLHVLGPLDDNDPEGEDYDEEADEGGDNENDLETRLKDLDIKQEE

SEQ ID NO: 19 - protein, 515 aa, Solanum tuberosum; identical to SEQ ID NO: 18

SEQ ID NO: 20 - DNA, 1236 bp, unidentified
  misc_feature 1..1236: note = metagenome purified from environmental niche
  source 1..1236: mol_type = unassigned DNA
atgtcctcgtcgtcttcctcgaaggttttctctatcctcggcaagaatctcaaggcgaacacggcagccgacctcgagcctcatttgagcgagctaaaggacttgaacgatgtcgaggaggttcactttggtggcaacagtctcggtgttgaggcctgtcaggctatcgctgaggtcttgaagacgaaacagtcgctcaaagtcgtcgacctcgcagatgtctttactggtcgattgatctctgaaatcccacaagccctctcagctctctgcgatgcccttcagtcccacacatcactcgtcgagctcgacctgtcggacaatgcgttcggtggtcgatgtgccgatgccatggtacccttccttaccaacaacacgcatttttcggtgttcaagctcaacaacaatggtctagggatctggggaggaacggtcgttagcaatgctttgctggagaatgccaagctctgcaagacccagggcaaggagagctctcttcgagtcatcgtgtgtggccgaaataggttggagaatgggtctgcacctcattgggccgaggcgtttgctacgcatggcaaattggtggaagtgaggatgccgcaaaacggcattcgcatggagggcatcaaagctatcgccgacggactggccaagtgtccgacattggaagtgcttgatttgcaggacaacacggctaccaagacaggaacacggagtattgtccgacacctctcaacttggcctaaacttcgaatactcaatctctcggactgtcttttgggttcggtcggcggtatcgctcttgcaaccgcattgtccactggctcgaacaagcacctcgaacagctcaaactgcaatatggcgagtttgacaagaggacggttgagatactgtcgacggcaattagccagcatttgccaaaattgacgacactcgaactgaatggaaaccgtttcgatgccgaagacgaatgcgttgagaccctgaagaaggcacttgagctacatgggaacgaggatgctttggacgaacttgacgatatggaggaggtggacgaggacgaagaggatgatgatgacgaggacgaggaggacgaagacgaggacaaggacactagcgccgacgatgggatcgatgcaggagctgctggagaagacgctctaccaccagtcacgaagaaggacgaggacgaacttgcggatctcctgtccaaggtccacgttcagcctagctga

SEQ ID NO: 21 - DNA, 1257 bp, unidentified
  misc_feature 1..1257: note = metagenome purified from environmental niche
  source 1..1257: mol_type = unassigned DNA
atgtcctcgtcgtcttcctcgaaggttttctctatcctcggcaagaatctcaaggcgaacacggcagccgacctcgagcctcatttgagcgagctaaaggacttgaacgatgtcgaggaggttcactttggtggcaacagtctcggtgttgaggcctgtcaggctatcgctgaggtcttgaagacgaaacagtcgctcaaagtcgtcgacctcgcagatgtctttactggtcgattgatctctgaaatcccacaagccctctcagctctctgcgatgcccttcagtcccacacatcactcgtcgagctcgacctgtcggacaatgcgttcggtggtcgatgtgccgatgccatggtacccttccttaccaacaacacgcatttttcggtgttcaagctcaacaacaatggtctagggatctggggaggaacggtcgttagcaatgctttgctggagaatgccaagctctgcaagacccagggcaaggagagctctcttcgagtcatcgtgtgtggccgaaataggttggagaatgggtctgcacctcattgggccgaggcgtttgctacgcatggcaaattggtggaagtgaggatgccgcaaaacggcattcgcatggagggcatcaaagctatcgccgacggactggccaagtgtccgacattggaagtgcttgatttgcaggacaacacggctaccaagacaggaacacggagtattgtccgacacctctcaacttggcctaaacttcgaatactcaatctctcggactgtcttttgggttcggtcggcggtatcgctcttgcaaccgcattgtccactggctcgaacaagcacctcgaacagctcaaactgcaatatggcgagtttgacaagaggacggttgagatactgtcgacggcaattagccagcatttgccaaaattgacgacactcgaactgaatggaaaccgtttcgatgccgaagacgaatgcgttgagaccctgaagaaggcacttgagctacatgggaacgaggatgctttggacgaacttgacgatatggaggaggtggacgaggacgaagaggatgatgatgacgaggacgaggaggacgaagacgaggacaaggacactagcgccgacgatgggatcgatgcaggagctgctggagaagacgctctaccaccagtcacgaagaaggacgaggacgaacttgcggatctcctgtccaaggtccacgttcagctagctgagtcccatgtgcgatcgctctag

SEQ ID NO: 22 - DNA, 1236 bp, unidentified; identical to SEQ ID NO: 20
  misc_feature 1..1236: note = metagenome purified from environmental niche
  source 1..1236: mol_type = unassigned DNA

SEQ ID NO: 23 - DNA, 1605 bp, Solanum tuberosum
  source 1..1605: mol_type = genomic DNA
atggattctgctggattctctatgaagctgtggcccccaagtttaagtaccaggctaatgctcgtggagagaatgaccaagaatcttattactccatccatcctatccaggaaatatggtcttttgagtaaagaagaagctgaagaggatgccaaacaaatagaagctctggcttttgattctgccaatcaacattttgacaaggagcctgatggcgatggaagttctgctgtgcaactttatgctaaggaatctagtaagctcatgttagaagttattaaaagaggccctcagactaaggaatctgcagaaggtattgtatctgagaaggttaaagcatctgatgaaactactatttttgatatatccaaaggtagacgggatttcctaagtgcagaagaggcgtctgagttattgaaaccattaagtgaaccagggaataattataaaaggatatgtttcagcaatagaagctttggcgtggatgctgctaaaattgcaggacctattttgtcctctcttaaagaccaattgacagaagttgacctttcagacttcgtagcaggtagaccagaggaagaagccctggaagtcatggaaatcttttcttctgctttggacgcctgtgacctgaggtacctcgatctttctaacaatgctttaggtgaaaaaggaataagggcatttggggcacttttaaagtcacaaaagaacttggaagaactctatttaatgaatgacggcatttcagaggaagcagcacaagcagtttgcgagctaatcccttcaactgataaacttcgtatccttcattttcacaataacatgacaggggatgagggtgcacttgctatttctaaacttgtgaagcattctcctgcattggaggatttccggtgctcatctacaagggtaggatctgaaggtggggttgctctttcacaagcacttggagaatgtaggaatttaaagaaggttgatttgcgtgacaacatgtttggtgtggaagctggaattgccttgagcaaagtactatcaatcttttccggcttaactgaaatttacttaagttatctgaatttggaggatgaggggtccatagctcttgctaatgtcctcaaggaatctgctccatcccttgaggttttggagatggatggaaatgacatcacagctaaagctgctcctgctctggcagcctgtattgctgcaaaacaatttcttaccacgttgaagttgggagagaatgaattgaaagatgagggtgctatcttaattgcaaaggcattggaggatggtcacggtcaattaactgaagtggatatgagtaccaatgcaataagaagagctggtgccaggtgcttggcacaagctgtagtgaacaaacctgggtttaaggtgctgaacatcaatggcaacttcatatctgatgaaggaattgatgaggtaaaagatatttttaagaattcacttcatgtgcttggacctttggatgacaatgatcctgaaggagaagattatgacgaagaggctgatgaaggaggcgataatgagaatgatttggagaccaggctcaaggatctcgatatcaagcaggaagaatga

SEQ ID NO: 24 - DNA, 1605 bp, Solanum tuberosum
  source 1..1605: mol_type = genomic DNA
atggattctgctggattctctatgaagctggcgcccccaagtttaagtactaggctaatgctcgtggagagaatgaccaagaatcttattactccatccatcctatccaggaaatatggtcttttgagtaaagaagaagctgaagaggatgccaaacaaatagaagctctggcttttgattctgccaatcaacattttgacaaggagcctgatggcgatggaagttctgctgtgcaactttatgctaaggaatctagtaagctcatgttagaagttattaaaagaggccctcagactaaggaatctgcagaaggtattgtatctgagaaggttaaagcatctgatgaaactactatttttgatatatccaaaggtagacgggatttcctaagtgcagaagaggcgtctgagttattgaaaccattaagtgaaccagggaataattataaaaggatatgtttcagcaatagaagctttggcgtggatgctgctaaaattgcaggacctattttgtcctctcttaaagaccaattgacagaagttgacctttcagacttcgtagcaggtagaccagaggaagaagccctggaagtcatggaaatcttttcttctgctttggacgcctgtgacctgaggtacctcgatctttctaacaatgctttaggtgaaaaaggaataagggcatttggggcacttttaaagtcacaaaagaacttggaagaactctatttaatgaatgacggcatttcagaggaagcagcacaagcagtttgcgagctaatcccttcaactgataaacttcgtatccttcattttcacaataacatgacaggggatgagggtgcacttgctatttctaaacttgtgaagcattctcctgcattggaggatttccggtgctcatctacaagggtaggatctgaaggtggggttgctctttcacaagcacttggagaatgtaggaatttaaagaaggttgatttgcgtgacaacatgtttggtgtggaagctggaattgccttgagcaaagtactatcaatcttttccggcttaactgaaatttacttaagttatctgaatttggaggatgaggggtccatagctcttgctaatgtcctcaaggaatctgctccatcccttgaggttttggagatggatggaaatgacatcacagctaaagctgctcctgctctggcagcctgtattgctgcaaaacaatttcttaccacgttgaagttgggagagaatgaattgaaagatgagggtgctatcttaattgcaaaggcattggaggatggtcacggtcaattaactgaagtggatatgagtaccaatgcaataagaagagctggtgccaggtgcttggcacaagctgtagtgaacaaacctgggtttaaggtgctgaacatcaatggcaacttcatatctgatgaaggaattgatgaggtaaaagatatttttaagaattcacttcatgtgcttggacctttggatgacaatgatcctgaaggagaagattatgacgaagaggctgatgaaggaggcgataatgagaatgatttggagaccaggctcaaggatctcgatatcaagcaggaagaatga

SEQ ID NO: 25 - DNA, 1605 bp, Solanum tuberosum
  source 1..1605: mol_type = genomic DNA
atggattctgctggattctctatgaagctggcgcccccaagtttaagtactaggctaatgctcgtggagagaatgaccaagaatcttattactccatccatcctatccaggaaatatggtcttttgagtaaagaagaagctgaagaggatgccaaacaaatagaagctctggcttttgattctgccaatcaacattttgacaaggagcctgatggcgatggaagttctgctgtgcaactttatgctaaggaatctagtaagctcatgttagaagttattaaaagaggccctcagactaaggaatctgcagaaggtattgtatctgagaaggttaaagcatctgatgaaactactatttttgatatatccaaaggtagacgggatttcctaagtgcagaagaggcgtctgagttattgaaaccattaagtgaaccagggaataattataaaaggatatgtttcagcaatagaagctttggcgtggatgctgctaaaattgcaggacctattttgtcctctcttaaagaccaattgacagaagttgacctttcagacttcgtagcaggtgcaccagaggaagaagccctggaagtcatggaaatcttttcttctgctttggacgcctgtgacctgaggtacctcgatctttctaacgctgctttaggtgaaaaaggaataagggcatttggggcacttttaaagtcacaaaagaacttggaagaactctatttaatgaatgacggcatttcagaggaagcagcacaagcagtttgcgagctaatcccttcaactgataaacttcgtatccttcattttcacaataacatgacaggggatgagggtgcacttgctatttctaaacttgtgaagcattctcctgcattggaggatttccggtgctcatctacaagggtaggatctgaaggtggggttgctctttcacaagcacttggagaatgtaggaatttaaagaaggttgatttgcgtgacaacatgtttggtgtggaagctggaattgccttgagcaaagtactatcaatcttttccggcttaactgaaatttacttaagttatctgaatttggaggatgaggggtccatagctcttgctaatgtcctcaaggaatctgctccatcccttgaggttttggagatggatggaaatgacatcacagctaaagctgctcctgctctggcagcctgtattgctgcaaaacaatttcttaccacgttgaagttgggagagaatgaattgaaagatgagggtgctatcttaattgcaaaggcattggaggatggtcacggtcaattaactgaagtggatatgagtaccaatgcaataagaagagctggtgccaggtgcttggcacaagctgtagtgaacaaacctgggtttaaggtgctgaacatcaatggcaacttcatatctgatgaaggaattgatgaggtaaaagatatttttaagaattcacttcatgtgcttggacctttggatgacaatgatcctgaaggagaagattatgacgaagaggctgatgaaggaggcgataatgagaatgatttggagaccaggctcaaggatctcgatatcaagcaggaagaatga

SEQ ID NO: 26 - DNA, 1242 bp, Solanum tuberosum
  source 1..1242: mol_type = genomic DNA
atgtccaaaggtagacgggatttcctaagtgcagaagaggcgtctgagttattgaaaccattaagtgaaccagggaataattataaaaggatatgtttcagcaatagaagctttggcgtggatgctgctaaaattgcaggacctattttgtcctctcttaaagaccaattgacagaagttgacctttcagacttcgtagcaggtagaccagaggaagaagccctggaagtcatggaaatcttttcttctgctttggacgcctgtgacctgaggtacctcgatctttctaacaatgctttaggtgaaaaaggaataagggcatttggggcacttttaaagtcacaaaagaacttggaagaactctatttaatgaatgacggcatttcagaggaagcagcacaagcagtttgcgagctaatcccttcaactgataaacttcgtatccttcattttcacaataacatgacaggggatgagggtgcacttgctatttctaaacttgtgaagcattctcctgcattggaggatttccggtgctcatctacaagggtaggatctgaaggtggggttgctctttcacaagcacttggagaatgtaggaatttaaagaaggttgatttgcgtgacaacatgtttggtgtggaagctggaattgccttgagcaaagtactatcaatcttttccggcttaactgaaatttacttaagttatctgaatttggaggatgaggggtccatagctcttgctaatgtcctcaaggaatctgctccatcccttgaggttttggagatggatggaaatgacatcacagctaaagctgctcctgctctggcagcctgtattgctgcaaaacaatttcttaccacgttgaagttgggagagaatgaattgaaagatgagggtgctatcttaattgcaaaggcattggaggatggtcacggtcaattaactgaagtggatatgagtaccaatgcaataagaagagctggtgccaggtgcttggcacaagctgtagtgaacaaacctgggtttaaggtgctgaacatcaatggcaacttcatatctgatgaaggaattgatgaggtaaaagatatttttaagaattcacttcatgtgcttggacctttggatgacaatgatcctgaaggagaagattatgacgaagaggctgatgaaggaggcgataatgagaatgatttggagaccaggctcaaggatctcgatatcaagcaggaagaatga

SEQ ID NO: 27 - DNA, 1242 bp, Solanum tuberosum
  source 1..1242: mol_type = genomic DNA
atgtccaaaggtagacgggatttcctaagtgcagaagaggcgtctgagttattgaaaccattaagtgaaccagggaataattataaaaggatatgtttcagcaatagaagctttggcgtggatgctgctaaaattgcaggacctattttgtcctctcttaaagaccaattgacagaagttgacctttcagacttcgtagcaggtgcaccagaggaagaagccctggaagtcatggaaatcttttcttctgctttggacgcctgtgacctgaggtacctcgatctttctaacgctgctttaggtgaaaaaggaataagggcatttggggcacttttaaagtcacaaaagaacttggaagaactctatttaatgaatgacggcatttcagaggaagcagcacaagcagtttgcgagctaatcccttcaactgataaacttcgtatccttcattttcacaataacatgacaggggatgagggtgcacttgctatttctaaacttgtgaagcattctcctgcattggaggatttccggtgctcatctacaagggtaggatctgaaggtggggttgctctttcacaagcacttggagaatgtaggaatttaaagaaggttgatttgcgtgacaacatgtttggtgtggaagctggaattgccttgagcaaagtactatcaatcttttccggcttaactgaaatttacttaagttatctgaatttggaggatgaggggtccatagctcttgctaatgtcctcaaggaatctgctccatcccttgaggttttggagatggatggaaatgacatcacagctaaagctgctcctgctctggcagcctgtattgctgcaaaacaatttcttaccacgttgaagttgggagagaatgaattgaaagatgagggtgctatcttaattgcaaaggcattggaggatggtcacggtcaattaactgaagtggatatgagtaccaatgcaataagaagagctggtgccaggtgcttggcacaagctgtagtgaacaaacctgggtttaaggtgctgaacatcaatggcaacttcatatctgatgaaggaattgatgaggtaaaagatatttttaagaattcacttcatgtgcttggacctttggatgacaatgatcctgaaggagaagattatgacgaagaggctgatgaaggaggcgataatgagaatgatttggagaccaggctcaaggatctcgatatcaagcaggaagaatga

SEQ ID NO: 28 - DNA, 726 bp, Solanum tuberosum
  source 1..726: mol_type = genomic DNA
atgttggaggatttccggtgctcatctacaagggtaggatctgaaggtggggttgctctttcacaagcacttggagaatgtaggaatttaaagaaggttgatttgcgtgacaacatgtttggtgtggaagctggaattgccttgagcaaagtactatcaatcttttccggcttaactgaaatttacttaagttatctgaatttggaggatgaggggtccatagctcttgctaatgtcctcaaggaatctgctccatcccttgaggttttggagatggatggaaatgacatcacagctaaagctgctcctgctctggcagcctgtattgctgcaaaacaatttcttaccacgttgaagttgggagagaatgaattgaaagatgagggtgctatcttaattgcaaaggcattggaggatggtcacggtcaattaactgaagtggatatgagtaccaatgcaataagaagagctggtgccaggtgcttggcacaagctgtagtgaacaaacctgggtttaaggtgctgaacatcaatggcaacttcatatctgatgaaggaattgatgaggtaaaagatatttttaagaattcacttcatgtgcttggacctttggatgacaatgatcctgaaggagaagattatgacgaagaggctgatgaaggaggcgataatgagaatgatttggagaccaggctcaaggatctcgatatcaagcaggaagaatga

SEQ ID NO: 29 - DNA, 687 bp, Solanum tuberosum
  source 1..687: mol_type = genomic DNA
atggaaggtggggttgctctttcacaagcacttggagaatgtaggaatttaaagaaggttgatttgcgtgacaacatgtttggtgtggaagctggaattgccttgagcaaagtactatcaatcttttccggcttaactgaaatttacttaagttatctgaatttggaggatgaggggtccatagctcttgctaatgtcctcaaggaatctgctccatcccttgaggttttggagatggatggaaatgacatcacagctaaagctgctcctgctctggcagcctgtattgctgcaaaacaatttcttaccacgttgaagttgggagagaatgaattgaaagatgagggtgctatcttaattgcaaaggcattggaggatggtcacggtcaattaactgaagtggatatgagtaccaatgcaataagaagagctggtgccaggtgcttggcacaagctgtagtgaacaaacctgggtttaaggtgctgaacatcaatggcaacttcatatctgatgaaggaattgatgaggtaaaagatatttttaagaattcacttcatgtgcttggacctttggatgacaatgatcctgaaggagaagattatgacgaagaggctgatgaaggaggcgataatgagaatgatttggagaccaggctcaaggatctcgatatcaagcaggaagaatga

SEQ ID NO: 30 - DNA, 1548 bp, Solanum tuberosum
  source 1..1548: mol_type = genomic DNA
atgctcgtggagagaatgaccaagaatcttattactccatccatcctatccaggaaatatggtcttttgagtaaagaagaagctgaagaggatgccaaacaaatagaagctctggcttttgattctgccaatcaacattttgacaaggagcctgatggcgatggaagttctgctgtgcaactttatgctaaggaatctagtaagctcatgttagaagttattaaaagaggccctcagactaaggaatctgcagaaggtattgtatctgagaaggttaaagcatctgatgaaactactatttttgatatatccaaaggtagacgggatttcctaagtgcagaagaggcgtctgagttattgaaaccattaagtgaaccagggaataattataaaaggatatgtttcagcaatagaagctttggcgtggatgctgctaaaattgcaggacctattttgtcctctcttaaagaccaattgacagaagttgacctttcagacttcgtagcaggtagaccagaggaagaagccctggaagtcatggaaatcttttcttctgctttggacgcctgtgacctgaggtacctcgatctttctaacaatgctttaggtgaaaaaggaataagggcatttggggcacttttaaagtcacaaaagaacttggaagaactctatttaatgaatgacggcatttcagaggaagcagcacaagcagtttgcgagctaatcccttcaactgataaacttcgtatccttcattttcacaataacatgacaggggatgagggtgcacttgctatttctaaacttgtgaagcattctcctgcattggaggatttccggtgctcatctacaagggtaggatctgaaggtggggttgctctttcacaagcacttggagaatgtaggaatttaaagaaggttgatttgcgtgacaacatgtttggtgtggaagctggaattgccttgagcaaagtactatcaatcttttccggcttaactgaaatttacttaagttatctgaatttggaggatgaggggtccatagctcttgctaatgtcctcaaggaatctgctccatcccttgaggttttggagatggatggaaatgacatcacagctaaagctgctcctgctctggcagcctgtattgctgcaaaacaatttcttaccacgttgaagttgggagagaatgaattgaaagatgagggtgctatcttaattgcaaaggcattggaggatggtcacggtcaattaactgaagtggatatgagtaccaatgcaataagaagagctggtgccaggtgcttggcacaagctgtagtgaacaaacctgggtttaaggtgctgaacatcaatggcaacttcatatctgatgaaggaattgatgaggtaaaagatatttttaagaattcacttcatgtgcttggacctttggatgacaatgatcctgaaggagaagattatgacgaagaggctgatgaaggaggcgataatgagaatgatttggagaccaggctcaaggatctcgatatcaagcaggaagaatga

SEQ ID NO: 31 - DNA, 1605 bp, Solanum tuberosum
  source 1..1605: mol_type = genomic DNA
atcgattctgctggattctctatcaagctgtggcccccaagtttaagtaccaggctaatgctcgtggagagaatgaccaagaatcttattactccatccatcctatccaggaaatatggtcttttgagtaaagaagaagctgaagaggatgccaaacaaatagaagctctggcttttgattctgccaatcaacattttgacaaggagcctgatggcgatggaagttctgctgtgcaactttatgctaaggaatctagtaagctcatgttagaagttattaaaagaggccctcagactaaggaatctgcagaaggtattgtatctgagaaggttaaagcatctgatgaaactactatttttgatatatccaaaggtagacgggatttcctaagtgcagaagaggcgtctgagttattgaaaccattaagtgaaccagggaataattataaaaggatatgtttcagcaatagaagctttggcgtggatgctgctaaaattgcaggacctattttgtcctctcttaaagaccaattgacagaagttgacctttcagacttcgtagcaggtagaccagaggaagaagccctggaagtcatggaaatcttttcttctgctttggacgcctgtgacctgaggtacctcgatctttctaacaatgctttaggtgaaaaaggaataagggcatttggggcacttttaaagtcacaaaagaacttggaagaactctatttaatgaatgacggcatttcagaggaagcagcacaagcagtttgcgagctaatcccttcaactgataaacttcgtatccttcattttcacaataacatgacaggggatgagggtgcacttgctatttctaaacttgtgaagcattctcctgcattggaggatttccggtgctcatctacaagggtaggatctgaaggtggggttgctctttcacaagcacttggagaatgtaggaatttaaagaaggttgatttgcgtgacaacatgtttggtgtggaagctggaattgccttgagcaaagtactatcaatcttttccggcttaactgaaatttacttaagttatctgaatttggaggatgaggggtccatagctcttgctaatgtcctcaaggaatctgctccatcccttgaggttttggagatggatggaaatgacatcacagctaaagctgctcctgctctggcagcctgtattgctgcaaaacaatttcttaccacgttgaagttgggagagaatgaattgaaagatgagggtgctatcttaattgcaaaggcattggaggatggtcacggtcaattaactgaagtggatatgagtaccaatgcaataagaagagctggtgccaggtgcttggcacaagctgtagtgaacaaacctgggtttaaggtgctgaacatcaatggcaacttcatatctgatgaaggaattgatgaggtaaaagatatttttaagaattcacttcatgtgcttggacctttggatgacaatgatcctgaaggagaagattatgacgaagaggctgatgaaggaggcgataatgagaatgatttggagaccaggctcaaggatctcgatatcaagcaggaagaatga

SEQ ID NO: 32 - protein, 160 aa, Solanum tuberosum
MSAPSLEVLEMDGNDITAKAAPALAACIAAKQFLTTLKLGENELKDEGAILIAKALEDGHGQLTEVDMSTNAIRRAGARCLAQAVVNKPGFKVLNINGNFISDEGIDEVKDIFKNSLHVLGPLDDNDPEGEDYDEEADEGGDNENDLETRLKDLDIKQEE

SEQ ID NO: 33 - protein, 61 aa, Solanum tuberosum
MISDEGIDEVKDIFKNSLHVLGPLDDNDPEGEDYDEEADEGGDNENDLETRLKDLDIKQEE

SEQ ID NO: 34 - protein, 42 aa, Solanum tuberosum
MLGPLDDNDPEGEDYDEEADEGGDNENDLETRLKDLDIKQEE

SEQ ID NO: 35 - DNA, 482 bp, Solanum tuberosum
  source 1..482: mol_type = genomic DNA
atgtctgctccatcccttgaggttttggagatggatggaaatgacatcacagctaaagctgctcctgctctggcagcctgtattgctgcaaaacaatttcttaccacgttgaagttgggagagaatgaattgaaagatgagggtgctatcttaattgcaaaggcattggaggatggtcacggtcaattaactgaagtggatatgagtaccaatgcaataagaagagctggtgccaggtgcttggcacaagctgtagtgaacaaacctgggtttaaggtgctgaacatcaatggcaacttcatatctgatgaaggaattgatgaggtaaaagatatttttaagaattcacttcatgtgcttggacctttggatgacaatgatcctgaaggagaagattatgacgaagaggctgatgaaggaggcgataatgagaatgatttggagaccaggctcaaggatctcgatatcaagcaggaagaatg

SEQ ID NO: 36 - DNA, 186 bp, Solanum tuberosum
  source 1..186: mol_type = genomic DNA
atgatatctgatgaaggaattgatgaggtaaaagatatttttaagaattcacttcatgtgcttggacctttggatgacaatgatcctgaaggagaagattatgacgaagaggctgatgaaggaggcgataatgagaatgatttggagaccaggctcaaggatctcgatatcaagcaggaagaatga

SEQ ID NO: 37 - DNA, 129 bp, Solanum tuberosum
  source 1..129: mol_type = genomic DNA
atgcttggacctttggatgacaatgatcctgaaggagaagattatgacgaagaggctgatgaaggaggcgataatgagaatgatttggagaccaggctcaaggatctcgatatcaagcaggaagaatga

SEQ ID NO: 38 - protein, 535 aa, Arabidopsis thaliana
MDHSAKTTQNRVLSVKMWPPSKSTRLMLVERMTKNITTPSIFSRKYGLLSVEEAEQDAKRIEDLAFATANKHFQNEPDGDGTSAVHVYAKESSKLMLDVIKRGPQEESEVEVSKDGDVFFDISGGSRAFIEEEEARDLLRPLADPRNSYTKIRFSNRSFGSEAAKFAASVLSSIKDQLTEVDLSDFVAGRPEAEALEVMNMFSSALEGSKLRYLNLSDNALGEKGIRAFASLINSQHDLEELYLMNDGISEDAARAVRELLPSTDKIRVLQFHNNMTGDEGATAIAEIVRECPSLEDFRCSSTRIGSEGGVALAEALEHCSHLKKLDLRDNMFGVEGGIALAKTLSVLTHLTEIYMSYLNLEDEGTEALSEALLKSAPSLEVLELAGNDITVKSTGNLAACIASKQSLAKLNLSENELKDEGTILIAKAVEGHDQLVEVDLSTNMIRRAGARALAQTVVKKNTFKLLNINGNFISEEGIDEVNDMFKDCLDKLVPLDDNDPEGEDFEDEDEEEEGEDGNELESKLGSLKIKQGEE

SEQ ID NO: 39 - protein, 414 aa, Arabidopsis thaliana
MSGGSRAFIEEEEARDLLRPLADPRNSYTKIRFSNRSFGSEAAKFAASVLSSIKDQLTEVDLSDFVAGRPEAEALEVMNMFSSALEGSKLRYLNLSDNALGEKGIRAFASLINSQHDLEELYLMNDGISEDAARAVRELLPSTDKIRVLQFHNNMTGDEGATAIAEIVRECPSLEDFRCSSTRIGSEGGVALAEALEHCSHLKKLDLRDNMFGVEGGIALAKTLSVLTHLTEIYMSYLNLEDEGTEALSEALLKSAPSLEVLELAGNDITVKSTGNLAACIASKQSLAKLNLSENELKDEGTILIAKAVEGHDQLVEVDLSTNMIRRAGARALAQTVVKKNTFKLLNINGNFISEEGIDEVNDMFKDCLDKLVPLDDNDPEGEDFEDEDEEEEGEDGNELESKLGSLKIKQGEE

SEQ ID NO: 40 - protein, 242 aa, Arabidopsis thaliana
MLEDFRCSSTRIGSEGGVALAEALEHCSHLKKLDLRDNMFGVEGGIALAKTLSVLTHLTEIYMSYLNLEDEGTEALSEALLKSAPSLEVLELAGNDITVKSTGNLAACIASKQSLAKLNLSENELKDEGTILIAKAVEGHDQLVEVDLSTNMIRRAGARALAQTVVKKNTFKLLNINGNFISEEGIDEVNDMFKDCLDKLVPLDDNDPEGEDFEDEDEEEEGEDGNELESKLGSLKIKQGEE

SEQ ID NO: 41 - protein, 229 aa, Arabidopsis thaliana
MEGGVALAEALEHCSHLKKLDLRDNMFGVEGGIALAKTLSVLTHLTEIYMSYLNLEDEGTEALSEALLKSAPSLEVLELAGNDITVKSTGNLAACIASKQSLAKLNLSENELKDEGTILIAKAVEGHDQLVEVDLSTNMIRRAGARALAQTVVKKNTFKLLNINGNFISEEGIDEVNDMFKDCLDKLVPLDDNDPEGEDFEDEDEEEEGEDGNELESKLGSLKIKQGEE

SEQ ID NO: 42 - protein, 509 aa, Arabidopsis thaliana
MLVERMTKNITTPSIFSRKYGLLSVEEAEQDAKRIEDLAFATANKHFQNEPDGDGTSAVHVYAKESSKLMLDVIKRGPQEESEVEVSKDGDVFFDISGGSRAFIEEEEARDLLRPLADPRNSYTKIRFSNRSFGSEAAKFAASVLSSIKDQLTEVDLSDFVAGRPEAEALEVMNMFSSALEGSKLRYLNLSDNALGEKGIRAFASLINSQHDLEELYLMNDGISEDAARAVRELLPSTDKIRVLQFHNNMTGDEGATAIAEIVRECPSLEDFRCSSTRIGSEGGVALAEALEHCSHLKKLDLRDNMFGVEGGIALAKTLSVLTHLTEIYMSYLNLEDEGTEALSEALLKSAPSLEVLELAGNDITVKSTGNLAACIASKQSLAKLNLSENELKDEGTILIAKAVEGHDQLVEVDLSTNMIRRAGARALAQTVVKKNTFKLLNINGNFISEEGIDEVNDMFKDCLDKLVPLDDNDPEGEDFEDEDEEEEGEDGNELESKLGSLKIKQGEE

SEQ ID NO: 43 - DNA, 1608 bp, Arabidopsis thaliana
  source 1..1608: mol_type = genomic DNA
atggatcattcagcgaaaaccacacagaaccgtgttttgtcagtgaagatgtggccaccgagtaagagtacccgtctcatgcttgttgagcggatgaccaagaacattaccaccccttccatcttctccaggaagtacggtcttttgtctgttgaagaggctgagcaagacgccaagcgcattgaagatttggcctttgctactgccaacaaacacttccagaacgagcctgatggtgatggcacttctgctgttcacgtctatgctaaagaatccagcaagctcatgcttgatgtcatcaaacgtggtccacaggaagaatccgaggttgaggtgagcaaggatggtgatgtcttttttgatatatctggtgggagcagagcttttattgaagaagaggaggcacgcgaccttttgaggcctctggctgacccgcgtaactcctataccaaaatccgttttagcaacaggagttttgggtctgaagcagccaaatttgctgcgagcgttttgtcttccatcaaggatcagctgacggaagtggatctctcggattttgttgcaggaagaccagaggctgaagcgcttgaagtcatgaatatgttttcatctgctttagagggttctaagcttaggtatctgaatctctcggacaatgctttgggagaaaagggaattagggcatttgcttctctcatcaattctcagcatgacttggaggagctctatctgatgaatgatggcatttccgaggatgctgcacgtgcagttcgtgagcttctcccttctactgacaaaatcagggttcttcagtttcataacaacatgacaggagatgaaggagcaactgccattgcagagattgtcagggagtgtccatctttggaagatttccggtgttcatctacaaggattggttcagaaggtggtgttgcactagctgaagcacttgaacactgtagccacttgaagaaacttgatcttcgtgacaacatgtttggagtcgaaggtggaattgccctggccaagactttatcagtcttgactcatttaaccgagatctatatgagttacctgaacttggaggatgagggaacagaggcactttctgaagctctacttaagtccgccccttctcttgaagttctagaattggctggtaacgacattactgtaaaatccactggaaatttggcagcttgcatcgcctccaaacagtctcttgcaaagctgaatttatctgagaatgaactcaaagacgaagggactatcttaattgcaaaggcagttgaaggtcatgatcagttagttgaagttgacctgagcaccaacatgatcagaagagctggggcaagggccttggcacaaactgttgtgaagaaaaatactttcaagcttctgaacatcaatggaaacttcatatctgaggaaggtattgacgaggtaaatgacatgtttaaggactgtctggacaagcttgttcctttggacgacaatgaccctgaaggagaagattttgaagatgaagacgaagaggaggaaggcgaagatggcaatgaattggaatcaaagcttggaagtctgaaaatcaagcaaggggaggaatga

SEQ ID NO: 44 - DNA, 1245 bp, Arabidopsis thaliana
  source 1..1245: mol_type = genomic DNA
atgtctggtgggagcagagcttttattgaagaagaggaggcacgcgaccttttgaggcctctggctgacccgcgtaactcctataccaaaatccgttttagcaacaggagttttgggtctgaagcagccaaatttgctgcgagcgttttgtcttccatcaaggatcagctgacggaagtggatctctcggattttgttgcaggaagaccagaggctgaagcgcttgaagtcatgaatatgttttcatctgctttagagggttctaagcttaggtatctgaatctctcggacaatgctttgggagaaaagggaattagggcatttgcttctctcatcaattctcagcatgacttggaggagctctatctgatgaatgatggcatttccgaggatgctgcacgtgcagttcgtgagcttctcccttctactgacaaaatcagggttcttcagtttcataacaacatgacaggagatgaaggagcaactgccattgcagagattgtcagggagtgtccatctttggaagatttccggtgttcatctacaaggattggttcagaaggtggtgttgcactagctgaagcacttgaacactgtagccacttgaagaaacttgatcttcgtgacaacatgtttggagtcgaaggtggaattgccctggccaagactttatcagtcttgactcatttaaccgagatctatatgagttacctgaacttggaggatgagggaacagaggcactttctgaagctctacttaagtccgccccttctcttgaagttctagaattggctggtaacgacattactgtaaaatccactggaaatttggcagcttgcatcgcctccaaacagtctcttgcaaagctgaatttatctgagaatgaactcaaagacgaagggactatcttaattgcaaaggcagttgaaggtcatgatcagttagttgaagttgacctgagcaccaacatgatcagaagagctggggcaagggccttggcacaaactgttgtgaagaaaaatactttcaagcttctgaacatcaatggaaacttcatatctgaggaaggtattgacgaggtaaatgacatgtttaaggactgtctggacaagcttgttcctttggacgacaatgaccctgaaggagaagattttgaagatgaagacgaagaggaggaaggcgaagatggcaatgaattggaatcaaagcttggaagtctgaaaatcaagcaaggggaggaatga

SEQ ID NO: 45 - DNA, 729 bp, Arabidopsis thaliana
  source 1..729: mol_type = genomic DNA
atgttggaagatttccggtgttcatctacaaggattggttcagaaggtggtgttgcactagctgaagcacttgaacactgtagccacttgaagaaacttgatcttcgtgacaacatgtttggagtcgaaggtggaattgccctggccaagactttatcagtcttgactcatttaaccgagatctatatgagttacctgaacttggaggatgagggaacagaggcactttctgaagctctacttaagtccgccccttctcttgaagttctagaattggctggtaacgacattactgtaaaatccactggaaatttggcagcttgcatcgcctccaaacagtctcttgcaaagctgaatttatctgagaatgaactcaaagacgaagggactatcttaattgcaaaggcagttgaaggtcatgatcagttagttgaagttgacctgagcaccaacatgatcagaagagctggggcaagggccttggcacaaactgttgtgaagaaaaatactttcaagcttctgaacatcaatggaaacttcatatctgaggaaggtattgacgaggtaaatgacatgtttaaggactgtctggacaagcttgttcctttggacgacaatgaccctgaaggagaagattttgaagatgaagacgaagaggaggaaggcgaagatggcaatgaattggaatcaaagcttggaagtctgaaaatcaagcaaggggaggaatga

SEQ ID NO: 46 - DNA, 690 bp, Arabidopsis thaliana
  source 1..690: mol_type = genomic DNA
atggaaggtggtgttgcactagctgaagcacttgaacactgtagccacttgaagaaacttgatcttcgtgacaacatgtttggagtcgaaggtggaattgccctggccaagactttatcagtcttgactcatttaaccgagatctatatgagttacctgaacttggaggatgagggaacagaggcactttctgaagctctacttaagtccgccccttctcttgaagttctagaattggctggtaacgacattactgtaaaatccactggaaatttggcagcttgcatcgcctccaaacagtctcttgcaaagctgaatttatctgagaatgaactcaaagacgaagggactatcttaattgcaaaggcagttgaaggtcatgatcagttagttgaagttgacctgagcaccaacatgatcagaagagctggggcaagggccttggcacaaactgttgtgaagaaaaatactttcaagcttctgaacatcaatggaaacttcatatctgaggaaggtattgacgaggtaaatgacatgtttaaggactgtctggacaagcttgttcctttggacgacaatgaccctgaaggagaagattttgaagatgaagacgaagaggaggaaggcgaagatggcaatgaattggaatcaaagcttggaagtctgaaaatcaagcaaggggaggaatga

SEQ ID NO: 47 - DNA, 1530 bp, Arabidopsis thaliana
  source 1..1530: mol_type = genomic DNA
atgcttgttgagcggatgaccaagaacattaccaccccttccatcttctccaggaagtacggtcttttgtctgttgaagaggctgagcaagacgccaagcgcattgaagatttggcctttgctactgccaacaaacacttccagaacgagcctgatggtgatggcacttctgctgttcacgtctatgctaaagaatccagcaagctcatgcttgatgtcatcaaacgtggtccacaggaagaatccgaggttgaggtgagcaaggatggtgatgtcttttttgatatatctggtgggagcagagcttttattgaagaagaggaggcacgcgaccttttgaggcctctggctgacccgcgtaactcctataccaaaatccgttttagcaacaggagttttgggtctgaagcagccaaatttgctgcgagcgttttgtcttccatcaaggatcagctgacggaagtggatctctcggattttgttgcaggaagaccagaggctgaagcgcttgaagtcatgaatatgttttcatctgctttagagggttctaagcttaggtatctgaatctctcggacaatgctttgggagaaaagggaattagggcatttgcttctctcatcaattctcagcatgacttggaggagctctatctgatgaatgatggcatttccgaggatgctgcacgtgcagttcgtgagcttctcccttctactgacaaaatcagggttcttcagtttcataacaacatgacaggagatgaaggagcaactgccattgcagagattgtcagggagtgtccatctttggaagatttccggtgttcatctacaaggattggttcagaaggtggtgttgcactagctgaagcacttgaacactgtagccacttgaagaaacttgatcttcgtgacaacatgtttggagtcgaaggtggaattgccctggccaagactttatcagtcttgactcatttaaccgagatctatatgagttacctgaacttggaggatgagggaacagaggcactttctgaagctctacttaagtccgccccttctcttgaagttctagaattggctggtaacgacattactgtaaaatccactggaaatttggcagcttgcatcgcctccaaacagtctcttgcaaagctgaatttatctgagaatgaactcaaagacgaagggactatcttaattgcaaaggcagttgaaggtcatgatcagttagttgaagttgacctgagcaccaacatgatcagaagagctggggcaagggccttggcacaaactgttgtgaagaaaaatactttcaagcttctgaacatcaatggaaacttcatatctgaggaaggtattgacgaggtaaatgacatgtttaaggactgtctggacaagcttgttcctttggacgacaatgaccctgaaggagaagattttgaagatgaagacgaagaggaggaaggcgaagatggcaatgaattggaatcaaagcttggaagtctgaaaatcaagcaaggggaggaatga

SEQ ID NO: 48 - protein, 535 aa, Camelina sativa
MDHSVKTTQNRVLSVKMWPPSKSTRLMLVERMTKNITTPSIFSRKYGLLSLQEAEDDAKRIEDLAFATANKHFQNEPDGDGTSAVQVYAKESSKLMLDVIKRGPQEESDVSAKQTGDVLFDISCGRRAFIDEDEARELLRPLTDPHNSFTKIRFSNRSFGSEAAKFAATVLSSIKDQLTEVDLSDVVAGRPEAEALEAMNMFSSALEGSKLRFLDLSDNALGEKGIRAFASLIKSQHDLEELYLMNDGISEDAARAVRELLPSTDKIRVLHFHNNMTGDEGAIAIAEIIRQCPSLEDFRCSSTRIGSEGGVALAEALEHCTHLKKLDLRDNMFGVEGGIALAKTLSVLTHLTEIYMSYLNLEDEGTEAIAEALLKSAPSLEVLELAGNDITVNSTGKLAACIASKQSLSKLNLSENELKDEGSILIAKAVEGHGQLLEVDLSTNMIRRAGARALAQTFVKKHTFKLLNINGNYISDEGIDEVNDMFKDCPDKLGLLDDNDPEGEDFEDESEEEEGEEDNELESKLGGLKIKQEEE

SEQ ID NO: 49 - protein, 414 aa, Camelina sativa
MSCGRRAFIDEDEARELLRPLTDPHNSFTKIRFSNRSFGSEAAKFAATVLSSIKDQLTEVDLSDVVAGRPEAEALEAMNMFSSALEGSKLRFLDLSDNALGEKGIRAFASLIKSQHDLEELYLMNDGISEDAARAVRELLPSTDKIRVLHFHNNMTGDEGAIAIAEIIRQCPSLEDFRCSSTRIGSEGGVALAEALEHCTHLKKLDLRDNMFGVEGGIALAKTLSVLTHLTEIYMSYLNLEDEGTEAIAEALLKSAPSLEVLELAGNDITVNSTGKLAACIASKQSLSKLNLSENELKDEGSILIAKAVEGHGQLLEVDLSTNMIRRAGARALAQTFVKKHTFKLLNINGNYISDEGIDEVNDMFKDCPDKLGLLDDNDPEGEDFEDESEEEEGEEDNELESKLGGLKIKQEEE

SEQ ID NO: 50 - protein, 242 aa, Camelina sativa
MLEDFRCSSTRIGSEGGVALAEALEHCTHLKKLDLRDNMFGVEGGIALAKTLSVLTHLTEIYMSYLNLEDEGTEAIAEALLKSAPSLEVLELAGNDITVNSTGKLAACIASKQSLSKLNLSENELKDEGSILIAKAVEGHGQLLEVDLSTNMIRRAGARALAQTFVKKHTFKLLNINGNYISDEGIDEVNDMFKDCPDKLGLLDDNDPEGEDFEDESEEEEGEEDNELESKLGGLKIKQEEE

SEQ ID NO: 51 - protein, 229 aa, Camelina sativa
MEGGVALAEALEHCTHLKKLDLRDNMFGVEGGIALAKTLSVLTHLTEIYMSYLNLEDEGTEAIAEALLKSAPSLEVLELAGNDITVNSTGKLAACIASKQSLSKLNLSENELKDEGSILIAKAVEGHGQLLEVDLSTNMIRRAGARALAQTFVKKHTFKLLNINGNYISDEGIDEVNDMFKDCPDKLGLLDDNDPEGEDFEDESEEEEGEEDNELESKLGGLKIKQEEE

SEQ ID NO: 52 - protein, 509 aa, Camelina sativa
MLVERMTKNITTPSIFSRKYGLLSLQEAEDDAKRIEDLAFATANKHFQNEPDGDGTSAVQVYAKESSKLMLDVIKRGPQEESDVSAKQTGDVLFDISCGRRAFIDEDEARELLRPLTDPHNSFTKIRFSNRSFGSEAAKFAATVLSSIKDQLTEVDLSDVVAGRPEAEALEAMNMFSSALEGSKLRFLDLSDNALGEKGIRAFASLIKSQHDLEELYLMNDGISEDAARAVRELLPSTDKIRVLHFHNNMTGDEGAIAIAEIIRQCPSLEDFRCSSTRIGSEGGVALAEALEHCTHLKKLDLRDNMFGVEGGIALAKTLSVLTHLTEIYMSYLNLEDEGTEAIAEALLKSAPSLEVLELAGNDITVNSTGKLAACIASKQSLSKLNLSENELKDEGSILIAKAVEGHGQLLEVDLSTNMIRRAGARALAQTFVKKHTFKLLNINGNYISDEGIDEVNDMFKDCPDKLGLLDDNDPEGEDFEDESEEEEGEEDNELESKLGGLKIKQEEE

SEQ ID NO: 53 - DNA, 1608 bp, Camelina sativa
  source 1..1608: mol_type = genomic DNA
atggatcattcagttaaaaccacgcagaaccgtgttttgtctgtcaagatgtggccacccagtaagagtacccgcctgatgcttgtcgagcgcatgaccaagaacatcaccaccccttccatcttctccagaaagtacggtcttttgagtctccaagaggctgaggatgatgccaagcgtattgaagacttggcttttgctaccgccaacaaacacttccagaacgagcctgatggtgatggcacttctgctgttcaggtctacgctaaagaatccagtaagctcatgcttgatgtcatcaagcgaggtccacaggaagaatctgatgtttcggctaagcagactggggatgtcttgtttgatatatcatgtggccgtagagcattcattgatgaagacgaggcacgtgagcttttgaggcctttgactgacccgcataactcctttaccaagatccgtttcagcaacaggagttttggctccgaagcagccaagtttgctgccactgttttgtcttccatcaaggatcagctcactgaagtggatctctccgatgttgttgcaggacgtccagaggctgaagcacttgaagccatgaatatgttttcatctgctttggagggttctaagcttaggtttttggatctctcggacaatgctcttggtgaaaaaggaattagagcgtttgcttctctcatcaagtctcagcatgacttagaggagctttatctgatgaatgatggcatttcagaggatgctgcaagagcagttcgtgagctacttccttctactgacaaaatcagggttcttcacttccataacaacatgacaggagatgaaggagctattgccattgctgagattatcaggcagtgtccatctttggaagacttccggtgttcatctacaaggattggttcagaaggtggtgttgcgctggctgaagcactcgaacactgtacccacttgaagaaacttgatcttcgtgacaacatgtttggagtcgaaggtggaattgccttggccaagactttgtcagtgttgactcatttaactgagatctatatgagttatctgaacttggaggatgagggaacagaagcgattgccgaagctctacttaagtccgccccttctcttgaagttctagaattggctggaaacgatattactgtcaattccactggaaagttggcagcttgcatcgcctcaaaacagtctctttcaaagctgaatctatctgagaatgaactcaaagacgaagggtctatcttaatcgcaaaggcagtagaaggtcatggtcagttacttgaagttgacttgagcaccaacatgatcagaagagctggggcaagagccttggcacaaacttttgtgaagaaacacactttcaagctgctgaacattaatggaaactatatatctgacgaaggtattgacgaggtaaatgatatgttcaaggactgtccggacaagcttggtcttttggacgacaatgaccctgaaggagaagattttgaagatgaaagcgaagaagaggaaggcgaagaagacaacgaactggaatcaaaactcggaggtctgaaaatcaagcaagaggaggaatag

SEQ ID NO: 54 - DNA, 1245 bp, Camelina sativa
  source 1..1245: mol_type = genomic DNA
atgtcatgtggccgtagagcattcattgatgaagacgaggcacgtgagcttttgaggcctttgactgacccgcataactcctttaccaagatccgtttcagcaacaggagttttggctccgaagcagccaagtttgctgccactgttttgtcttccatcaaggatcagctcactgaagtggatctctccgatgttgttgcaggacgtccagaggctgaagcacttgaagccatgaatatgttttcatctgctttggagggttctaagcttaggtttttggatctctcggacaatgctcttggtgaaaaaggaattagagcgtttgcttctctcatcaagtctcagcatgacttagaggagctttatctgatgaatgatggcatttcagaggatgctgcaagagcagttcgtgagctacttccttctactgacaaaatcagggttcttcacttccataacaacatgacaggagatgaaggagctattgccattgctgagattatcaggcagtgtccatctttggaagacttccggtgttcatctacaaggattggttcagaaggtggtgttgcgctggctgaagcactcgaacactgtacccacttgaagaaacttgatcttcgtgacaacatgtttggagtcgaaggtggaattgccttggccaagactttgtcagtgttgactcatttaactgagatctatatgagttatctgaacttggaggatgagggaacagaagcgattgccgaagctctacttaagtccgccccttctcttgaagttctagaattggctggaaacgatattactgtcaattccactggaaagttggcagcttgcatcgcctcaaaacagtctctttcaaagctgaatctatctgagaatgaactcaaagacgaagggtctatcttaatcgcaaaggcagtagaaggtcatggtcagttacttgaagttgacttgagcaccaacatgatcagaagagctggggcaagagccttggcacaaacttttgtgaagaaacacactttcaagctgctgaacattaatggaaactatatatctgacgaaggtattgacgaggtaaatgatatgttcaaggactgtccggacaagcttggtcttttggacgacaatgaccctgaaggagaagattttgaagatgaaagcgaagaagaggaaggcgaagaagacaacgaactggaatcaaaactcggaggtctgaaaatcaagcaagaggaggaatag

SEQ ID NO: 55 - DNA, 729 bp, Camelina sativa
  source 1..729: mol_type = genomic DNA
atgttggaagacttccggtgttcatctacaaggattggttcagaaggtggtgttgcgctggctgaagcactcgaacactgtacccacttgaagaaacttgatcttcgtgacaacatgtttggagtcgaaggtggaattgccttggccaagactttgtcagtgttgactcatttaactgagatctatatgagttatctgaacttggaggatgagggaacagaagcgattgccgaagctctacttaagtccgccccttctcttgaagttctagaattggctggaaacgatattactgtcaattccactggaaagttggcagcttgcatcgcctcaaaacagtctctttcaaagctgaatctatctgagaatgaactcaaagacgaagggtctatcttaatcgcaaaggcagtagaaggtcatggtcagttacttgaagttgacttgagcaccaacatgatcagaagagctggggcaagagccttggcacaaacttttgtgaagaaacacactttcaagctgctgaacattaatggaaactatatatctgacgaaggtattgacgaggtaaatgatatgttcaaggactgtccggacaagcttggtcttttggacgacaatgaccctgaaggagaagattttgaagatgaaagcgaagaagaggaaggcgaagaagacaacgaactggaatcaaaactcggaggtctgaaaatcaagcaagaggaggaatag

SEQ ID NO: 56 - DNA, 690 bp, Camelina sativa
  source 1..690: mol_type = genomic DNA
atggaaggtggtgttgcgctggctgaagcactcgaacactgtacccacttgaagaaacttgatcttcgtgacaacatgtttggagtcgaaggtggaattgccttggccaagactttgtcagtgttgactcatttaactgagatctatatgagttatctgaacttggaggatgagggaacagaagcgattgccgaagctctacttaagtccgccccttctcttgaagttctagaattggctggaaacgatattactgtcaattccactggaaagttggcagcttgcatcgcctcaaaacagtctctttcaaagctgaatctatctgagaatgaactcaaagacgaagggtctatcttaatcgcaaaggcagtagaaggtcatggtcagttacttgaagttgacttgagcaccaacatgatcagaagagctggggcaagagccttggcacaaacttttgtgaagaaacacactttcaagctgctgaacattaatggaaactatatatctgacgaaggtattgacgaggtaaatgatatgttcaaggactgtccggacaagcttggtcttttggacgacaatgaccctgaaggagaagattttgaagatgaaagcgaagaagaggaaggcgaagaagacaacgaactggaatcaaaactcggaggtctgaaaatcaagcaagaggaggaatag

SEQ ID NO: 57 - DNA, 1530 bp, Camelina sativa
  source 1..1530: mol_type = genomic DNA
atgcttgtcgagcgcatgaccaagaacatcaccaccccttccatcttctccagaaagtacggtcttttgagtctccaagaggctgaggatgatgccaagcgtattgaagacttggcttttgctaccgccaacaaacacttccagaacgagcctgatggtgatggcacttctgctgttcaggtctacgctaaagaatccagtaagctcatgcttgatgtcatcaagcgaggtccacaggaagaatctgatgtttcggctaagcagactggggatgtcttgtttgatatatcatgtggccgtagagcattcattgatgaagacgaggcacgtgagcttttgaggcctttgactgacccgcataactcctttaccaagatccgtttcagcaacaggagttttggctccgaagcagccaagtttgctgccactgttttgtcttccatcaaggatcagctcactgaagtggatctctccgatgttgttgcaggacgtccagaggctgaagcacttgaagccatgaatatgttttcatctgctttggagggttctaagcttaggtttttggatctctcggacaatgctcttggtgaaaaaggaattagagcgtttgcttctctcatcaagtctcagcatgacttagaggagctttatctgatgaatgatggcatttcagaggatgctgcaagagcagttcgtgagctacttccttctactgacaaaatcagggttcttcacttccataacaacatgacaggagatgaaggagctattgccattgctgagattatcaggcagtgtccatctttggaagacttccggtgttcatctacaaggattggttcagaaggtggtgttgcgctggctgaagcactcgaacactgtacccacttgaagaaacttgatcttcgtgacaacatgtttggagtcgaaggtggaattgccttggccaagactttgtcagtgttgactcatttaactgagatctatatgagttatctgaacttggaggatgagggaacagaagcgattgccgaagctctacttaagtccgccccttctcttgaagttctagaattggctggaaacgatattactgtcaattccactggaaagttggcagcttgcatcgcctcaaaacagtctctttcaaagctgaatctatctgagaatgaactcaaagacgaagggtctatcttaatcgcaaaggcagtagaaggtcatggtcagttacttgaagttgacttgagcaccaacatgatcagaagagctggggcaagagccttggcacaaacttttgtgaagaaacacactttcaagctgctgaacattaatggaaactatatatctgacgaaggtattgacgaggtaaatgatatgttcaaggactgtccggacaagcttggtcttttggacgacaatgaccctgaaggagaagattttgaagatgaaagcgaagaagaggaaggcgaagaagacaacgaactggaatcaaaactcggaggtctgaaaatcaagcaagaggaggaatag

SEQ ID NO: 58 - protein, 533 aa, Glycine max
MDSTSQAYQHRPLTIKLWPPSQSTRLMLVERMTKNLTTPSIFSRKYGLLSKEEAEEDAKHIEDVAFATATQHFEKEPDGDGSSAVQIYAKESSKLMLEALKRGPKVKEDGELITEKSGSTAETVFDISGGRRDFISGEEAAEVLKPLTGPKYYTKICFSNRSFGLDAARVAEPILLSVKDQLKEVDLSDFIAGRPEAEALEVMTIFSSALEGSVLRYLNLSNNAMGEKGVRAFRSLLKSQTSLEELYLMNDGISEEAAKAVSELLPSTEKLRVLHFHNNMTGDEGAIAIAEIVKHSPALEDFRCSSTRVGSDGGVALAEALGACKHLRKLDLRDNMFGEEAGVALSKVIPAFTDLTEIYLSYLNLEDDGAEALANALKESAPSLEILDLAGNDITAKASASVAACISSKQFLTKLNLSENELKDEGAVLISKALEGGHGQLIEVDLSTNSITWSGAKLVAEAVVGKPGFKLLNINANFISDEGIDELKNIFKNSPDMLGPLDENNPDGEDTDEEAEEDADHDELESKLKGLGI

SEQ ID NO: 59 - protein, 407 aa, Glycine max
MSGGRRDFISGEEAAEVLKPLTGPKYYTKICFSNRSFGLDAARVAEPILLSVKDQLKEVDLSDFIAGRPEAEALEVMTIFSSALEGSVLRYLNLSNNAMGEKGVRAFRSLLKSQTSLEELYLMNDGISEEAAKAVSELLPSTEKLRVLHFHNNMTGDEGAIAIAEIVKHSPALEDFRCSSTRVGSDGGVALAEALGACKHLRKLDLRDNMFGEEAGVALSKVIPAFTDLTEIYLSYLNLEDDGAEALANALKESAPSLEILDLAGNDITAKASASVAACISSKQFLTKLNLSENELKDEGAVLISKALEGGHGQLIEVDLSTNSITWSGAKLVAEAVVGKPGFKLLNINANFISDEGIDELKNIFKNSPDMLGPLDENNPDGEDTDEEAEEDADHDELESKLKGLGI

SEQ ID NO: 60 - protein, 236 aa, Glycine max
MLEDFRCSSTRVGSDGGVALAEALGACKHLRKLDLRDNMFGEEAGVALSKVIPAFTDLTEIYLSYLNLEDDGAEALANALKESAPSLEILDLAGNDITAKASASVAACISSKQFLTKLNLSENELKDEGAVLISKALEGGHGQLIEVDLSTNSITWSGAKLVAEAVVGKPGFKLLNINANFISDEGIDELKNIFKNSPDMLGPLDENNPDGEDTDEEAEEDADHDELESKLKGLGI

SEQ ID NO: 61 - protein, 223 aa, Glycine max
MDGGVALAEALGACKHLRKLDLRDNMFGEEAGVALSKVIPAFTDLTEIYLSYLNLEDDGAEALANALKESAPSLEILDLAGNDITAKASASVAACISSKQFLTKLNLSENELKDEGAVLISKALEGGHGQLIEVDLSTNSITWSGAKLVAEAVVGKPGFKLLNINANFISDEGIDELKNIFKNSPDMLGPLDENNPDGEDTDEEAEEDADHDELESKLKGLGI

SEQ ID NO: 62 - protein, 507 aa, Glycine max
MLVERMTKNLTTPSIFSRKYGLLSKEEAEEDAKHIEDVAFATATQHFEKEPDGDGSSAVQIYAKESSKLMLEALKRGPKVKEDGELITEKSGSTAETVFDISGGRRDFISGEEAAEVLKPLTGPKYYTKICFSNRSFGLDAARVAEPILLSVKDQLKEVDLSDFIAGRPEAEALEVMTIFSSALEGSVLRYLNLSNNAMGEKGVRAFRSLLKSQTSLEELYLMNDGISEEAAKAVSELLPSTEKLRVLHFHNNMTGDEGAIAIAEIVKHSPALEDFRCSSTRVGSDGGVALAEALGACKHLRKLDLRDNMFGEEAGVALSKVIPAFTDLTEIYLSYLNLEDDGAEALANALKESAPSLEILDLAGNDITAKASASVAACISSKQFLTKLNLSENELKDEGAVLISKALEGGHGQLIEVDLSTNSITWSGAKLVAEAVVGKPGFKLLNINANFISDEGIDELKNIFKNSPDMLGPLDENNPDGEDTDEEAEEDADHDELESKLKGLGI

SEQ ID NO: 63 - protein, 507 aa, Glycine max; identical to SEQ ID NO: 62

SEQ ID NO: 64 - DNA, 1602 bp, Glycine max
  source 1..1602: mol_type = genomic DNA
atggattctacaagtcaggcataccaacatcgcccccttaccatcaaattatggcctcctagccagagtactaggctaatgcttgtagagcggatgaccaagaaccttacaactccgtcaattttctctagaaagtatggccttcttagcaaggaagaggctgaggaggatgcgaaacacattgaggatgtagcttttgctactgcaacccaacattttgagaaggagcctgatggtgatgggagttctgctgtgcaaatttatgctaaggaatcaagtaagctcatgttggaggctctgaaaagaggcccaaaagtgaaggaggatggagagttgataactgaaaagtctggttcaactgctgaaactgtttttgacatatctgggggtcgcagagactttattagtggggaggaagctgcagaagttttgaaaccattgacgggaccaaagtattataccaagatatgttttagcaatagaagttttggcttggatgctgcccgtgttgctgaacccattctattatcagttaaggatcaattgaaagaggtggatctttcagatttcattgctggaagaccagaagcagaggctcttgaagtgatgactatattttcttctgcactggaaggtagtgttttgaggtatctgaacctgtcaaataatgccatgggtgaaaaaggggttagagcttttcggtcactcctaaaatcacaaactagcttggaggagctttatttgatgaatgatggtatatcagaggaagctgcaaaagcagtttctgaattgcttccttccactgagaagcttagggttcttcatttccataacaacatgactggggatgaaggggcaattgctattgctgaaattgtgaaacattccccagctttggaagattttcggtgctcatcaaccagagtaggctctgatggtggagttgcccttgctgaagcacttggggcttgtaaacatttgaggaagcttgacttgcgtgacaacatgtttggagaagaagctggagttgctctaagtaaagttatacctgcatttacagatcttacagagatatacctcagttatttgaacctggaggatgatggtgcagaagcccttgctaatgccctcaaggaatctgcaccatcactggaaattttggatttggctgggaatgacattactgcaaaagcttctgcttctgtggctgcctgtatatcatcaaaacaattcctcaccaagttaaatttatctgagaatgaactgaaggatgaaggtgcagttttgatcagcaaggcactggaaggaggtcacggccaattaattgaagttgatttgagcactaactcgatcacatggtcaggagctaagctggtggctgaagctgttgtgggaaaaccaggttttaagttgcttaacattaatgctaacttcatttctgatgaaggaattgatgagttgaaaaatatattcaaaaactcacctgatatgctgggtcctttggatgagaataatcctgatggagaagacactgatgaggaggctgaagaagatgctgatcatgatgaattggaatcaaaactgaagggccttgggatttag

SEQ ID NO: 65 - DNA, 1224 bp, Glycine max
  source 1..1224: mol_type = genomic DNA
atgtctgggggtcgcagagactttattagtggggaggaagctgcagaagttttgaaaccattgacgggaccaaagtattataccaagatatgttttagcaatagaagttttggcttggatgctgcccgtgttgctgaacccattctattatcagttaaggatcaattgaaagaggtggatctttcagatttcattgctggaagaccagaagcagaggctcttgaagtgatgactatattttcttctgcactggaaggtagtgttttgaggtatctgaacctgtcaaataatgccatgggtgaaaaaggggttagagcttttcggtcactcctaaaatcacaaactagcttggaggagctttatttgatgaatgatggtatatcagaggaagctgcaaaagcagtttctgaattgcttccttccactgagaagcttagggttcttcatttccataacaacatgactggggatgaaggggcaattgctattgctgaaattgtgaaacattccccagctttggaagattttcggtgctcatcaaccagagtaggctctgatggtggagttgcccttgctgaagcacttggggcttgtaaacatttgaggaagcttgacttgcgtgacaacatgtttggagaagaagctggagttgctctaagtaaagttatacctgcatttacagatcttacagagatatacctcagttatttgaacctggaggatgatggtgcagaagcccttgctaatgccctcaaggaatctgcaccatcactggaaattttggatttggctgggaatgacattactgcaaaagcttctgcttctgtggctgcctgtatatcatcaaaacaattcctcaccaagttaaatttatctgagaatgaactgaaggatgaaggtgcagttttgatcagcaaggcactggaaggaggtcacggccaattaattgaagttgatttgagcactaactcgatcacatggtcaggagctaagctggtggctgaagctgttgtgggaaaaccaggttttaagttgcttaacattaatgctaacttcatttctgatgaaggaattgatgagttgaaaaatatattcaaaaactcacctgatatgctgggtcctttggatgagaataatcctgatggagaagacactgatgaggaggctgaagaagatgctgatcatgatgaattggaatcaaaactgaagggccttgggatttag

SEQ ID NO: 66 - DNA, 711 bp, Glycine max
  source 1..711: mol_type = genomic DNA
atgttggaagattttcggtgctcatcaaccagagtaggctctgatggtggagttgcccttgctgaagcacttggggcttgtaaacatttgaggaagcttgacttgcgtgacaacatgtttggagaagaagctggagttgctctaagtaaagttatacctgcatttacagatcttacagagatatacctcagttatttgaacctggaggatgatggtgcagaagcccttgctaatgccctcaaggaatctgcaccatcactggaaattttggatttggctgggaatgacattactgcaaaagcttctgcttctgtggctgcctgtatatcatcaaaacaattcctcaccaagttaaatttatctgagaatgaactgaaggatgaaggtgcagttttgatcagcaaggcactggaaggaggtcacggccaattaattgaagttgatttgagcactaactcgatcacatggtcaggagctaagctggtggctgaagctgttgtgggaaaaccaggttttaagttgcttaacattaatgctaacttcatttctgatgaaggaattgatgagttgaaaaatatattcaaaaactcacctgatatgctgggtcctttggatgagaataatcctgatggagaagacactgatgaggaggctgaagaagatgctgatcatgatgaattggaatcaaaactgaagggccttgggatttag

SEQ ID NO: 67 - DNA, 672 bp, Glycine max
  source 1..672: mol_type = genomic DNA
atggatggtggagttgcccttgctgaagcacttggggcttgtaaacatttgaggaagcttgacttgcgtgacaacatgtttggagaagaagctggagttgctctaagtaaagttatacctgcatttacagatcttacagagatatacctcagttatttgaacctggaggatgatggtgcagaagcccttgctaatgccctcaaggaatctgcaccatcactggaaattttggatttggctgggaatgacattactgcaaaagcttctgcttctgtggctgcctgtatatcatcaaaacaattcctcaccaagttaaatttatctgagaatgaactgaaggatgaaggtgcagttttgatcagcaaggcactggaaggaggtcacggccaattaattgaagttgatttgagcactaactcgatcacatggtcaggagctaagctggtggctgaagctgttgtgggaaaaccaggttttaagttgcttaacattaatgctaacttcatttctgatgaaggaattgatgagttgaaaaatatattcaaaaactcacctgatatgctgggtcctttggatgagaataatcctgatggagaagacactgatgaggaggctgaagaagatgctgatcatgatgaattggaatcaaaactgaagggccttgggatttag

SEQ ID NO: 68 - DNA, 1524 bp, Glycine max
  source 1..1524: mol_type = genomic DNA
atgcttgtagagcggatgaccaagaaccttacaactccgtcaattttctctagaaagtatggccttcttagcaaggaagaggctgaggaggatgcgaaacacattgaggatgtagcttttgctactgcaacccaacattttgagaaggagcctgatggtgatgggagttctgctgtgcaaatttatgctaaggaatcaagtaagctcatgttggaggctctgaaaagaggcccaaaagtgaaggaggatggagagttgataactgaaaagtctggttcaactgctgaaactgtttttgacatatctgggggtcgcagagactttattagtggggaggaagctgcagaagttttgaaaccattgacgggaccaaagtattataccaagatatgttttagcaatagaagttttggcttggatgctgcccgtgttgctgaacccattctattatcagttaaggatcaattgaaagaggtggatctttcagatttcattgctggaagaccagaagcagaggctcttgaagtgatgactatattttcttctgcactggaaggtagtgttttgaggtatctgaacctgtcaaataatgccatgggtgaaaaaggggttagagcttttcggtcactcctaaaatcacaaactagcttggaggagctttatttgatgaatgatggtatatcagaggaagctgcaaaagcagtttctgaattgcttccttccactgagaagcttagggttcttcatttccataacaacatgactggggatgaaggggcaattgctattgctgaaattgtgaaacattccccagctttggaagattttcggtgctcatcaaccagagtaggctctgatggtggagttgcccttgctgaagcacttggggcttgtaaacatttgaggaagcttgacttgcgtgacaacatgtttggagaagaagctggagttgctctaagtaaagttatacctgcatttacagatcttacagagatatacctcagttatttgaacctggaggatgatggtgcagaagcccttgctaatgccctcaaggaatctgcaccatcactggaaattttggatttggctgggaatgacattactgcaaaagcttctgcttctgtggctgcctgtatatcatcaaaacaattcctcaccaagttaaatttatctgagaatgaactgaaggatgaaggtgcagttttgatcagcaaggcactggaaggaggtcacggccaattaattgaagttgatttgagcactaactcgatcacatggtcaggagctaagctggtggctgaagctgttgtgggaaaaccaggttttaagttgcttaacattaatgctaacttcatttctgatgaaggaattgatgagttgaaaaatatattcaaaaactcacctgatatgctgggtcctttggatgagaataatcctgatggagaagacactgatgaggaggctgaagaagatgctgatcatgatgaattggaatcaaaactgaagggccttgggatttag

SEQ ID NO: 69 - DNA, 1602 bp, Glycine max
  source 1..1602: mol_type = genomic DNA
atcgattctacaagtcaggcataccaacatcgcccccttaccatcaaattatggcctcctagccagagtactaggctaatgcttgtagagcggatgaccaagaaccttacaactccgtcaattttctctagaaagtatggccttcttagcaaggaagaggctgaggaggatgcgaaacacattgaggatgtagcttttgctactgcaacccaacattttgagaaggagcctgatggtgatgggagttctgctgtgcaaatttatgctaaggaatcaagtaagctcatgttggaggctctgaaaagaggcccaaaagtgaaggaggatggagagttgataactgaaaagtctggttcaactgctgaaactgtttttgacatatctgggggtcgcagagactttattagtggggaggaagctgcagaagttttgaaaccattgacgggaccaaagtattataccaagatatgttttagcaatagaagttttggcttggatgctgcccgtgttgctgaacccattctattatcagttaaggatcaattgaaagaggtggatctttcagatttcattgctggaagaccagaagcagaggctcttgaagtgatgactatattttcttctgcactggaaggtagtgttttgaggtatctgaacctgtcaaataatgccatgggtgaaaaaggggttagagcttttcggtcactcctaaaatcacaaactagcttggaggagctttatttgatgaatgatggtatatcagaggaagctgcaaaagcagtttctgaattgcttccttccactgagaagcttagggttcttcatttccataacaacatgactggggatgaaggggcaattgctattgctgaaattgtgaaacattccccagctttggaagattttcggtgctcatcaaccagagtaggctctgatggtggagttgcccttgctgaagcacttggggcttgtaaacatttgaggaagcttgacttgcgtgacaacatgtttggagaagaagctggagttgctctaagtaaagttatacctgcatttacagatcttacagagatatacctcagttatttgaacctggaggatgatggtgcagaagcccttgctaatgccctcaaggaatctgcaccatcactggaaattttggatttggctgggaatgacattactgcaaaagcttctgcttctgtggctgcctgtatatcatcaaaacaattcctcaccaagttaaatttatctgagaatgaactgaaggatgaaggtgcagttttgatcagcaaggcactggaaggaggtcacggccaattaattgaagttgatttgagcactaactcgatcacatggtcaggagctaagctggtggctgaagctgttgtgggaaaaccaggttttaagttgcttaacattaatgctaacttcatttctgatgaaggaattgatgagttgaaaaatatattcaaaaactcacctgatatgctgggtcctttggatgagaataatcctgatggagaagacactgatgaggaggctgaagaagatgctgatcatgatgaattggaatcaaaactgaagggccttgggatttag

SEQ ID NO: 70 - protein, 544 aa, Oryza sativa
MDSTKQDFQPRTFSIKLWPPSESTRLMLVERMTKNLSTESIFSRKYGLLGKEEAHDNAKRIEEVCFASADEHFKEEPDGDGSSAVQLYAKETSKLMLEVLKRGPRTTVEPEVPVADTPLEPADSVFDISGGKRAFIEADEAKELLSPLIKPGNAYKRICFSNRSFGIGAANVAGPILESIKKQLTEVDISDFVAGRPEDEALDVMRIFSKALEGAVLRYLNISDNALGEKGVRAFEELLKSQDNLEELYVMNDGISEEAAQALSELIPSTEKLKILHFHNNMTGDEGAMFIAEMVKRSPNLESFRCSATRIGSDGGVALAEALGTCTRLKKLDLRDNLFGVEAGLALSKTLSKLPDLVELYLSDLNLENKGTVAIINTLKQSAPQLEVLEMAGNEINAKASQALAECLTAMQSLKKLTLAENELKDDGAVVIAKSLEDGHQDLKELDVSTNMLQRVGARCFAQAIANKPGFVQLNINGNFISDEGIDEVKDILKSGENSVEVLGPLDENDPEGEAEDDEEEEEEEEDDGELDSKLQSLKVEQDD

SEQ ID NO: 71 - protein, 417 aa, Oryza sativa
MSGGKRAFIEADEAKELLSPLIKPGNAYKRICFSNRSFGIGAANVAGPILESIKKQLTEVDISDFVAGRPEDEALDVMRIFSKALEGAVLRYLNISDNALGEKGVRAFEELLKSQDNLEELYVMNDGISEEAAQALSELIPSTEKLKILHFHNNMTGDEGAMFIAEMVKRSPNLESFRCSATRIGSDGGVALAEALGTCTRLKKLDLRDNLFGVEAGLALSKTLSKLPDLVELYLSDLNLENKGTVAIINTLKQSAPQLEVLEMAGNEINAKASQALAECLTAMQSLKKLTLAENELKDDGAVVIAKSLEDGHQDLKELDVSTNMLQRVGARCFAQAIANKPGFVQLNINGNFISDEGIDEVKDILKSGENSVEVLGPLDENDPEGEAEDDEEEEEEEEDDGELDSKLQSLKVEQDD

SEQ ID NO: 72 - protein, 245 aa, Oryza sativa
MLESFRCSATRIGSDGGVALAEALGTCTRLKKLDLRDNLFGVEAGLALSKTLSKLPDLVELYLSDLNLENKGTVAIINTLKQSAPQLEVLEMAGNEINAKASQALAECLTAMQSLKKLTLAENELKDDGAVVIAKSLEDGHQDLKELDVSTNMLQRVGARCFAQAIANKPGFVQLNINGNFISDEGIDEVKDILKSGENSVEVLGPLDENDPEGEAEDDEEEEEEEEDDGELDSKLQSLKVEQDD

SEQ ID NO: 73 - protein, 232 aa, Oryza sativa
MDGGVALAEALGTCTRLKKLDLRDNLFGVEAGLALSKTLSKLPDLVELYLSDLNLENKGTVAIINTLKQSAPQLEVLEMAGNEINAKASQALAECLTAMQSLKKLTLAENELKDDGAVVIAKSLEDGHQDLKELDVSTNMLQRVGARCFAQAIANKPGFVQLNINGNFISDEGIDEVKDILKSGENSVEVLGPLDENDPEGEAEDDEEEEEEEEDDGELDSKLQSLKVEQDD

SEQ ID NO: 74 - protein, 518 aa, Oryza sativa
MLVERMTKNLSTESIFSRKYGLLGKEEAHDNAKRIEEVCFASADEHFKEEPDGDGSSAVQLYAKETSKLMLEVLKRGPRTTVEPEVPVADTPLEPADSVFDISGGKRAFIEADEAKELLSPLIKPGNAYKRICFSNRSFGIGAANVAGPILESIKKQLTEVDISDFVAGRPEDEALDVMRIFSKALEGAVLRYLNISDNALGEKGVRAFEELLKSQDNLEELYVMNDGISEEAAQALSELIPSTEKLKILHFHNNMTGDEGAMFIAEMVKRSPNLESFRCSATRIGSDGGVALAEALGTCTRLKKLDLRDNLFGVEAGLALSKTLSKLPDLVELYLSDLNLENKGTVAIINTLKQSAPQLEVLEMAGNEINAKASQALAECLTAMQSLKKLTLAENELKDDGAVVIAKSLEDGHQDLKELDVSTNMLQRVGARCFAQAIANKPGFVQLNINGNFISDEGIDEVKDILKSGENSVEVLGPLDENDPEGEAEDDEEEEEEEEDDGELDSKLQSLKVEQDD

SEQ ID NO: 75 - protein, 518 aa, Oryza sativa; identical to SEQ ID NO: 74

SEQ ID NO: 76 - DNA, 1635 bp, Oryza sativa
  source 1..1635: mol_type = genomic DNA
atggattcgactaagcaagatttccaaccccggacattctccatcaaactgtggccaccaagtgaaagcactcgtctcatgcttgtggagaggatgaccaagaacctttccacagagtccatattttctcgcaagtacggccttttgggcaaggaagaggctcatgataatgctaaaaggatcgaagaggtatgctttgcctctgcagacgagcatttcaaggaggagcctgatggggatgggagttctgctgtccagctgtacgcaaaggaaactagcaagttgatgctggaagttctgaaaagagggccaaggaccactgtggaaccagaagtacctgttgctgatacacctcttgagcctgctgattctgtatttgatatatctggtggcaagcgtgcgtttattgaggcagatgaagcgaaggaacttttgagcccgcttatcaaaccaggaaatgcatataaacggatttgcttcagcaacaggagctttggtattggtgctgccaatgttgctggacctattctggaatcaatcaaaaagcaactcacagaggtggatatatcagattttgttgctggcagacctgaggatgaagcccttgatgtgatgcgcatcttctcaaaagctttagaaggcgctgtactgagatacttgaacatctctgataatgctttaggtgagaagggtgtcagagcatttgaagagctcctgaaatcacaggacaacctggaagaactatatgtgatgaatgatggtatatcagaggaagccgcacaagctctttctgaacttattccttcaactgagaagcttaagatcctccacttccacaacaatatgacgggggatgaaggtgctatgtttattgctgagatggttaaacgttctcctaatctagagagtttcaggtgctcagcaacaaggataggatctgatggtggggtggctctggctgaggcattagggacttgtactcgtctgaagaaacttgatctcagggacaacttgtttggagttgaggcagggttggctctcagcaaaaccctttcaaaacttcctgatcttgttgagctgtatctcagtgatctcaatcttgagaacaagggcactgtagcaattatcaataccctcaaacagtcagcaccccagttggaggtccttgaaatggctgggaatgaaattaacgccaaagcatcccaagctttagcagaatgcctgacagcaatgcaatcacttaagaagttgaccttggcagagaatgaactgaaggatgatggtgctgtggtgattgcaaaatcactggaagatggacaccaggatctgaaggagcttgatgttagcacgaatatgctgcagagggtgggagctcggtgctttgcccaggcaatcgcaaacaagccaggttttgtgcaactgaacataaacgggaatttcatctcggacgaagggattgatgaggtgaaagatattctgaagagtggtgagaactctgtggaggtgcttggtccactagatgagaacgaccctgagggggaggctgaagatgatgaggaagaggaagaggaggaagaggatgacggtgagctggactcgaagcttcagagcctgaaggttgagcaggacgattag

SEQ ID NO: 77 - DNA, 1254 bp, Oryza sativa
  source 1..1254: mol_type = genomic DNA
atgtctggtggcaagcgtgcgtttattgaggcagatgaagcgaaggaacttttgagcccgcttatcaaaccaggaaatgcatataaacggatttgcttcagcaacaggagctttggtattggtgctgccaatgttgctggacctattctggaatcaatcaaaaagcaactcacagaggtggatatatcagattttgttgctggcagacctgaggatgaagcccttgatgtgatgcgcatcttctcaaaagctttagaaggcgctgtactgagatacttgaacatctctgataatgctttaggtgagaagggtgtcagagcatttgaagagctcctgaaatcacaggacaacctggaagaactatatgtgatgaatgatggtatatcagaggaagccgcacaagctctttctgaacttattccttcaactgagaagcttaagatcctccacttccacaacaatatgacgggggatgaaggtgctatgtttattgctgagatggttaaacgttctcctaatctagagagtttcaggtgctcagcaacaaggataggatctgatggtggggtggctctggctgaggcattagggacttgtactcgtctgaagaaacttgatctcagggacaacttgtttggagttgaggcagggttggctctcagcaaaaccctttcaaaacttcctgatcttgttgagctgtatctcagtgatctcaatcttgagaacaagggcactgtagcaattatcaataccctcaaacagtcagcaccccagttggaggtccttgaaatggctgggaatgaaattaacgccaaagcatcccaagctttagcagaatgcctgacagcaatgcaatcacttaagaagttgaccttggcagagaatgaactgaaggatgatggtgctgtggtgattgcaaaatcactggaagatggacaccaggatctgaaggagcttgatgttagcacgaatatgctgcagagggtgggagctcggtgctttgcccaggcaatcgcaaacaagccaggttttgtgcaactgaacataaacgggaatttcatctcggacgaagggattgatgaggtgaaagatattctgaagagtggtgagaactctgtggaggtgcttggtccactagatgagaacgaccctgagggggaggctgaagatgatgaggaagaggaagaggaggaagaggatgacggtgagctggactcgaagcttcagagcctgaaggttgagcaggacgattag

SEQ ID NO: 78 - DNA, 738 bp, Oryza sativa
  source 1..738: mol_type = genomic DNA
atgctagagagtttcaggtgctcagcaacaaggataggatctgatggtggggtggctctggctgaggcattagggacttgtactcgtctgaagaaacttgatctcagggacaacttgtttggagttgaggcagggttggctctcagcaaaaccctttcaaaacttcctgatcttgttgagctgtatctcagtgatctcaatcttgagaacaagggcactgtagcaattatcaataccctcaaacagtcagcaccccagttggaggtccttgaaatggctgggaatgaaattaacgccaaagcatcccaagctttagcagaatgcctgacagcaatgcaatcacttaagaagttgaccttggcagagaatgaactgaaggatgatggtgctgtggtgattgcaaaatcactggaagatggacaccaggatctgaaggagcttgatgttagcacgaatatgctgcagagggtgggagctcggtgctttgcccaggcaatcgcaaacaagccaggttttgtgcaactgaacataaacgggaatttcatctcggacgaagggattgatgaggtgaaagatattctgaagagtggtgagaactctgtggaggtgcttggtccactagatgagaacgaccctgagggggaggctgaagatgatgaggaagaggaagaggaggaagaggatgacggtgagctggactcgaagcttcagagcctgaaggttgagcaggacgattag

SEQ ID NO: 79 - DNA, 699 bp, Oryza sativa
  source 1..699: mol_type = genomic DNA
atggatggtggggtggctctggctgaggcattagggacttgtactcgtctgaagaaacttgatctcagggacaacttgtttggagttgaggcagggttggctctcagcaaaaccctttcaaaacttcctgatcttgttgagctgtatctcagtgatctcaatcttgagaacaagggcactgtagcaattatcaataccctcaaacagtcagcaccccagttggaggtccttgaaatggctgggaatgaaattaacgccaaagcatcccaagctttagcagaatgcctgacagcaatgcaatcacttaagaagttgaccttggcagagaatgaactgaaggatgatggtgctgtggtgattgcaaaatcactggaagatggacaccaggatctgaaggagcttgatgttagcacgaatatgctgcagagggtgggagctcggtgctttgcccaggcaatcgcaaacaagccaggttttgtgcaactgaacataaacgggaatttcatctcggacgaagggattgatgaggtgaaagatattctgaagagtggtgagaactctgtggaggtgcttggtccactagatgagaacgaccctgagggggaggctgaagatgatgaggaagaggaagaggaggaagaggatgacggtgagctggactcgaagcttcagagcctgaaggttgagcaggacgattag

SEQ ID NO: 80 - DNA, 1557 bp, Oryza sativa
  source 1..1557: mol_type = genomic DNA
atgcttgtggagaggatgaccaagaacctttccacagagtccatattttctcgcaagtacggccttttgggcaaggaagaggctcatgataatgctaaaaggatcgaagaggtatgctttgcctctgcagacgagcatttcaaggaggagcctgatggggatgggagttctgctgtccagctgtacgcaaaggaaactagcaagttgatgctggaagttctgaaaagagggccaaggaccactgtggaaccagaagtacctgttgctgatacacctcttgagcctgctgattctgtatttgatatatctggtggcaagcgtgcgtttattgaggcagatgaagcgaaggaacttttgagcccgcttatcaaaccaggaaatgcatataaacggatttgcttcagcaacaggagctttggtattggtgctgccaatgttgctggacctattctggaatcaatcaaaaagcaactcacagaggtggatatatcagattttgttgctggcagacctgaggatgaagcccttgatgtgatgcgcatcttctcaaaagctttagaaggcgctgtactgagatacttgaacatctctgataatgctttaggtgagaagggtgtcagagcatttgaagagctcctgaaatcacaggacaacctggaagaactatatgtgatgaatgatggtatatcagaggaagccgcacaagctctttctgaacttattccttcaactgagaagcttaagatcctccacttccacaacaatatgacgggggatgaaggtgctatgtttattgctgagatggttaaacgttctcctaatctagagagtttcaggtgctcagcaacaaggataggatctgatggtggggtggctctggctgaggcattagggacttgtactcgtctgaagaaacttgatctcagggacaacttgtttggagttgaggcagggttggctctcagcaaaaccctttcaaaacttcctgatcttgttgagctgtatctcagtgatctcaatcttgagaacaagggcactgtagcaattatcaataccctcaaacagtcagcaccccagttggaggtccttgaaatggctgggaatgaaattaacgccaaagcatcccaagctttagcagaatgcctgacagcaatgcaatcacttaagaagttgaccttggcagagaatgaactgaaggatgatggtgctgtggtgattgcaaaatcactggaagatggacaccaggatctgaaggagcttgatgttagcacgaatatgctgcagagggtgggagctcggtgctttgcccaggcaatcgcaaacaagccaggttttgtgcaactgaacataaacgggaatttcatctcggacgaagggattgatgaggtgaaagatattctgaagagtggtgagaactctgtggaggtgcttggtccactagatgagaacgaccctgagggggaggctgaagatgatgaggaagaggaagaggaggaagaggatgacggtgagctggactcgaagcttcagagcctgaaggttgagcaggacgattag

SEQ ID NO: 81 - DNA, 1635 bp, Oryza sativa
  source 1..1635: mol_type = genomic DNA
atcgattcgactaagcaagatttccaaccccggacattctccatcaaactgtggccaccaagtgaaagcactcgtctcatgcttgtggagaggatgaccaagaacctttccacagagtccatattttctcgcaagtacggccttttgggcaaggaagaggctcatgataatgctaaaaggatcgaagaggtatgctttgcctctgcagacgagcatttcaaggaggagcctgatggggatgggagttctgctgtccagctgtacgcaaaggaaactagcaagttgatgctggaagttctgaaaagagggccaaggaccactgtggaaccagaagtacctgttgctgatacacctcttgagcctgctgattctgtatttgatatatctggtggcaagcgtgcgtttattgaggcagatgaagcgaaggaacttttgagcccgcttatcaaaccaggaaatgcatataaacggatttgcttcagcaacaggagctttggtattggtgctgccaatgttgctggacctattctggaatcaatcaaaaagcaactcacagaggtggatatatcagattttgttgctggcagacctgaggatgaagcccttgatgtgatgcgcatcttctcaaaagctttagaaggcgctgtactgagatacttgaacatctctgataatgctttaggtgagaagggtgtcagagcatttgaagagctcctgaaatcacaggacaacctggaagaactatatgtgatgaatgatggtatatcagaggaagccgcacaagctctttctgaacttattccttcaactgagaagcttaagatcctccacttccacaacaatatgacgggggatgaaggtgctatgtttattgctgagatggttaaacgttctcctaatctagagagtttcaggtgctcagcaacaaggataggatctgatggtggggtggctctggctgaggcattagggacttgtactcgtctgaagaaacttgatctcagggacaacttgtttggagttgaggcagggttggctctcagcaaaaccctttcaaaacttcctgatcttgttgagctgtatctcagtgatctcaatcttgagaacaagggcactgtagcaattatcaataccctcaaacagtcagcaccccagttggaggtccttgaaatggctgggaatgaaattaacgccaaagcatcccaagctttagcagaatgcctgacagcaatgcaatcacttaagaagttgaccttggcagagaatgaactgaaggatgatggtgctgtggtgattgcaaaatcactggaagatggacaccaggatctgaaggagcttgatgttagcacgaatatgctgcagagggtgggagctcggtgctttgcccaggcaatcgcaaacaagccaggttttgtgcaactgaacataaacgggaatttcatctcggacgaagggattgatgaggtgaaagatattctgaagagtggtgagaactctgtggaggtgcttggtccactagatgagaacgaccctgagggggaggctgaagatgatgaggaagaggaagaggaggaagaggatgacggtgagctggactcgaagcttcagagcctgaaggttgagcaggacgattag

SEQ ID NO: 82 - protein, 138 aa, unidentified
  REGION 1..138: note = metagenome purified from environmental niche
MGSNKHLEQLKLQYGEFDKRTVEILSTAISQHLPKLTTLELNGNRFDAEDECVETLKKALELHGNEDALDELDDMEEVDEDEEDDDDEDEEDEDEDKDTSADDGIDAGAAGEDALPPVTKKDEDELADLLSKVHVQPS

SEQ ID NO: 83 - protein, 107 aa, unidentified
  REGION 1..107: note = metagenome purified from environmental niche
MLPKLTTLELNGNRFDAEDECVETLKKALELHGNEDALDELDDMEEVDEDEEDDDDEDEEDEDEDKDTSADDGIDAGAAGEDALPPVTKKDEDELADLLSKVHVQPS

SEQ ID NO: 84 - protein, 81 aa, unidentified
  REGION 1..81: note = metagenome purified from environmental niche
MALELHGNEDALDELDDMEEVDEDEEDDDDEDEEDEDEDKDTSADDGIDAGAAGEDALPPVTKKDEDELADLLSKVHVQPS

SEQ ID NO: 85 - DNA, 417 bp, unidentified
  misc_feature 1..417: note = metagenome purified from environmental niche
  source 1..417: mol_type = unassigned DNA
atgggctcgaacaagcacctcgaacagctcaaactgcaatatggcgagtttgacaagaggacggttgagatactgtcgacggcaattagccagcatttgccaaaattgacgacactcgaactgaatggaaaccgtttcgatgccgaagacgaatgcgttgagaccctgaagaaggcacttgagctacatgggaacgaggatgctttggacgaacttgacgatatggaggaggtggacgaggacgaagaggatgatgatgacgaggacgaggaggacgaagacgaggacaaggacactagcgccgacgatgggatcgatgcaggagctgctggagaagacgctctaccaccagtcacgaagaaggacgaggacgaacttgcggatctcctgtccaaggtccacgttcagcctagctga

SEQ ID NO: 86 - DNA, 324 bp, unidentified
  misc_feature 1..324: note = metagenome purified from environmental niche
  source 1..324: mol_type = unassigned DNA
atgttgccaaaattgacgacactcgaactgaatggaaaccgtttcgatgccgaagacgaatgcgttgagaccctgaagaaggcacttgagctacatgggaacgaggatgctttggacgaacttgacgatatggaggaggtggacgaggacgaagaggatgatgatgacgaggacgaggaggacgaagacgaggacaaggacactagcgccgacgatgggatcgatgcaggagctgctggagaagacgctctaccaccagtcacgaagaaggacgaggacgaacttgcggatctcctgtccaaggtccacgttcagcctagctga

SEQ ID NO: 87 - DNA, 246 bp, unidentified
  misc_feature 1..246: note = metagenome purified from environmental niche
  source 1..246: mol_type = unassigned DNA
atggcacttgagctacatgggaacgaggatgctttggacgaacttgacgatatggaggaggtggacgaggacgaagaggatgatgatgacgaggacgaggaggacgaagacgaggacaaggacactagcgccgacgatgggatcgatgcaggagctgctggagaagacgctctaccaccagtcacgaagaaggacgaggacgaacttgcggatctcctgtccaaggtccacgttcagcctagctga

SEQ ID NO: 88 - DNA, 816 bp, unidentified
  misc_feature 1..816: note = metagenome purified from environmental niche
  source 1..816: mol_type = unassigned DNA
atgggcgggaccgtcgtgagtaacgccctgcttgagaacgcaaaattgtgtaaaacacagggaaaagagagttccctcagagtaatagtttgcggtaggaataggcttgaaaatgggtccgctcctcattgggctgaagccttcgccactcatggtaagcttgttgaagttagaatgcctcaaaacgggatcagaatggaaggcattaaagctattgccgacggcctggcaaagtgcccaacccttgaggtcctggatcttcaagacaatactgctaccaaaactggaacacgttctatcgtaagacacctttctacttggcctaagcttcgtatcctcaatttgtctgattgtctgctcggttctgtaggaggcatagcacttgcaacagcactgtcaactggctccaataaacacttggagcagctgaaattgcagtatggcgaatttgataagcgcacagttgagatcctctccactgccattagtcagcacctgcctaaattgaccacacttgagttgaacggcaatagattcgatgcagaggacgaatgtgtggaaaccctgaaaaaggccttggaactccacggaaacgaagatgctctggatgagctcgacgacatggaggaagtggatgaggatgaggaagatgatgacgatgaggacgaggaggacgaggacgaggacaaggacacatctgcagacgacggtattgacgctggggcagctggtgaggacgctcttcccccagtgaccaagaaagacgaggatgtactggccgatttgctgagtaaagttcatgtgcaacctagttga

SEQ ID NO: 89 - protein, 533 aa, Arachis hypogaea
MDSTAQTYQHRPLSIKLWPPSQGTRLMLAERMTKNLTTPSIFSRKYGLLSKEEAEEDAKQIEEIAFTTATQHFEKEPDGDGSFAVQIYAKESSKLMLDVLKRGPRVKEDGVLVSEKVTVTGETVFDISGGRRAFIDAEEATELLEPLRAPNSYTKICFSNRSFGLEAAQVAEPILLSIKNQLKEVDLSDFIAGRPEAEALEVMTIFSSALEGCVLRYLNLSNNAMGEKGVRAFRSLLKSQNNLEELYLMNDGISEEAAKAVSELIPSTEKLKVLHFHNNMTGDEGAIAISEIVKRSAALEDFRCSSTRVGSDGGVVLAEALGTCKHLKKLDLRDNMFGVEAGVALSKAVLGFVDLTEIYLSYLNLEDDGAEALANALKESAPSLEVLDMAGNDITTNVAASLAECISSKQFLAKLNLSENELKDEGAILISKALEEGHDQLLEVDLSTNSITRSGATVLAKAVVKKPRFKLLNINANFISDEGIEELKDIFKSLPDMLGPLDENDPEGEDIDEEAEEDADRDELESKLKSLEI

SEQ ID NO: 90 - protein, 535 aa, Brassica napus
MDHSVKTPPVLSVKMWPPSKSTRLMLVDRMTKNITTPSIFSRKYGLLSLQEAEEDAKRIEELAFATANKHFQSEPDADGTSAVHVYAKESSKLMLEVIKRGPQQVDSEAEANNDGDALFDLSGGRRAFIDLEEARELLRPLAEPNNSYTKIRFSNRSFGSEAAKFAVGVLSSVKDQLTEVDLSDFVAGRPEAEALEVMTMFSSALEGSNLRSLNLSDNALGEKGIRAFASLIKSQRGLEELYLMNDGISEDAARAVRELLPSTGEIRVLQFHNNMTGDEGAIAIAEIVKQCPSLEDFRCSSTRIGSDGGVALAEALASCSRLKKLDLRDNMFGVEGGVALANTLSVLNELTEIYMSYLNLEDEGTEALAEALIKSAPRLEVIELAGNDITVKSTGKLAACIALKESLSKLNLSENELKDEGTIIIAKALEEGHDQLAEVDMSTNMMRRAGARALAQTVLKKPTLKLLNINGNFISEEGVDEVNDMFKDSADKLGPLDDNDPEGEDFEDEDEEEEGEEDNALEAKLGGMKIKQEEE

SEQ ID NO: 91 - protein, 536 aa, Beta vulgaris
MDAMQTNPERRPFSIKLWPPSQNTRIMLVERMTSNLSTPTIFTQKYGSLSKEEAEENAKQIEEVAFTAANQFYEKEPDGDGSSAVQLYAKECSKLILEALKRGPVTKVVREVPISELVHESLFDISKGPRAFIEAEEAEELLAPLKDPSNNYTKICFSNRSFGLGAARVAEPILASIKDKLKEVDLSDFIAGRPEVEALEVMNMFSAALEGAVLKSLNLSNNALGEKGVLAFGALLKSQSNLEELYLMNDGISEEAARAVNELVPSTEKLRVLQFHNNMTGDEGALAISEVVKRSPLLEDFRCSSTRVASEGGVALSEALEGCVHLKKLDLRDNMFGVESGVALSKALLKHENLLEAYLSYLNLEDEGTIAIANALKDSAPLLEVLELAGNDVTPAATPALAACLTSKQHLLKVNLAENELKDEGALHVSKALEGHTKLKELDMNTNAIRRAGARVLANMVVDKPDFEMLNINGNFISDDGIDDVNEIFKKSPEKLGPLDENDPEGGDEDEKESEDGDGEENELEFELKNLDVGEE

SEQ ID NO: 92 - protein, 533 aa, Coffea arabica
MDSKTFSMKLWPPSQSTRLMLVERMTKNLSTPSVLSRKYGLLSKEEAEEDAKQIEVVAFASASQHFEKESDGDGSSAVQLYAKESSKLMLDVIRRGPRTKEDGNFIESERVRVSHETVFDISGGRRAFIDAKEAEELLKPLGDLHNKFTKICFSNTSFGLDSAHVAEPILSSLRDQLTEIDLSDFIAGRPETEALEVMTIFSSALEGSNLRYLNLSNNALGEKGVRAFAGLLKSQHNLEELYLMNDGISEEAARAVCELIPSTDRLRVLHFHNNMTGDEGAVAISELVKHSPLLEDFRCSSARVGSEGGVALAEALGSCTSLKKLDLRDNMFGVESGIALSKVLRRFSDLTEIYLSYLNLEDEGSLALANALTESVPSLEVLEMAGNDITAEAAPALAACVAAKQFLTKLNLAENELKDEGAILIAKALEEGHGQLNEVDLSTNSIRRAGARCLAQAVVSKPGFKLLNINGNFISDEGVDEVKEVFENSSCVLGPLDDNDPEGEDYDEEAEEGDDDEGELESKLKGLEIKQEE

SEQ ID NO: 93 - protein, 538 aa, Daucus carota
MDTAVRNRTISIKLWPPSQSTRLMLVERMTKNLTTPSILSRKYGLLDIAEAAEDAKKIETVAYEAANQHFEKEPDGDGSSAVQLYAKESSKLMLEVIKRGPQPKENGKAIESEEVAVSHNTFFDISGGRRAFIEHEEATELLKPLGEPGSKFTKICFSNRSFGLDAARVAEEFLSSLKDQLKEVDLSDFVAGRPETEALEVMTIFSSVLDGCDLRFLNLSDNALGEKGVRAFGSLLKSQRNLEELYLMNDGISEEAAQAVRELIPSTEKLRVLQFHNNMTGDEGAIAISQLVKNSPLLEDFRCSSTRVGSEGGVALAEALEICTHLKKLDLRDNMFGVEAGLALSKTLPGFGNLIEVYLSYLNLEDEGVIALAEALKKSANSLQVLEMAGNDITVKAAPALASLIAGGDSITKLNLSENELKDDGAILIAQALEQGHSQLKEVDLSTNAIRRVGARALAQTVVAKPEFQLLNIDGNFISDEGVDEVKVVFKSSPAKLGPLDDNDPEGEDNNEEDEDAASDEDDELGSKLQGLEIKHDD

SEQ ID NO: 94 - protein, 538 aa, Gossypium hirsutum
MESGTQAFQQRSLSVKLWPPSHGTRLMLVERMTKNLTTPSIFSRKYGLLSNEEAEEDAKKIEELAFAAADQHYKKEPDGDGSSAVQVYAKESSRLMLEAIKRGPTVKDEEIADKTSIHETVFDISGGRRAFIDAEEAEEILKPLREPGNSYTKICFSNRSFGLDAANVAAPLLSSVKQQLTEVDLSDFIAGRPESEALEVMNLFSSVLEGCQLRYLNLSNNALGEKGVRAFGALLKSQNNLEELYLINDGISEEAAQAVSELIPSTKKLKVLHFHNNMTGDEGAIAISDIVKRSPALEDFRCSSTRVGTDGGVALAEALKTCTRLKKLDLRDNMFGVGAGVALSKALSLFAGLTEVYLSYLNLEDDGAEALANALKESAPSLEVLDMAGNDITAKGAASLAACIASKQFLSKLNLAENELKDEGAILIGKSLGEGHGQLNEIDMSTNAIRRAGARLLAQVVVKIPGFKLLNINGNFISDEGIDEIKELFKSSPDMLGSLEDNDPEGEDYEDEDEDEENAEEGNELESKLKDLKIEQGE

SEQ ID NO: 95 - protein, 552 aa, Hordeum vulgare
MDSTAQDFQPRTFTIKLWPPSESTRLMLVERMTKNLSDESIFSRKYGILGKEEAHENAKRIEEVCFASADEHFKKEPDGDGSSAVQLYAKETSKLMLEVLKKGPRTTAEPEAPVIDAPLEPAATVFDISGGKRAFIEAEEAKELLSPLTKPGNSYKRICFSNRSFGIGAANVAGPILESIKSQLTEVDISDFVAGRPEDEALDVMRIFSKALAGSVLRYLNISDNALGEKGVRAFTELLKSQGDLEELYVMNDGISGEAAKALSELIPSTEKLKVLHFHNNMTGDEGAMPIAEMVKRSPNLESFRCSATRIGSDGGVALAEALGTCTRLKKLDIRDNLFGVEAGVALSKTLPKLGGLVELYLSDLNLENEGTIAIVDVLKQSAPQLEVLEMAGNEITAKAAKAVAECLTAMQSLKKLTLAENELKDAGAVTIAKSLQEGHPGLEELDVSTNLFQRSGARCFAQAVANKPGFALLNINSNFISDEGIDEVKEILKGGENSLEVLGPLDENDPEGDPEDDEEEEDDEDEEKDGNDNGDGGLGSKLQGLKVEEED

SEQ ID NO: 96 - protein, 545 aa, Lactuca sativa
MESTAPNQLRSIKLWPPSHGTRLVLVQRIVKNLTTPSILSRKYGILSKEEAEEEAKQIESSAFAIANQHFEKEPDGDGGSAVQTYAKESSKLMVEAVKRGPKPKTDQEPEPIPDTVTPHHETFFDISGGKRAFIEADEAQELLKPLSEPGNKYTKICFSNRSFGLDAARIAGPILSSLKEQLTEVDLSDFIAGRPEADALEVMTIFASALEGSNLRYLNLSNNALGEKGVRAFEKLLASQSNLEELYLMNDGISEEAAKAVCELIPSTNKLKVLHFHNNMTGDEGAIAISGLVKESPILEDFRCSSTRVDSEGGVALSKALGTCPNLKKIDLRDNMFGIEAGIALSKAIPVFTSLTEVYFSYLNLEDEGTLALVNAIKDSDSPLEVLEMAGNDITSESASALAAVITAKKNTLIKLVLSENELKDEGVIVIANSLKEEFGQLTEVDLSTNGIRTVGARALSQAVVGKPGFKLLNINGNFLSDEGVDNVKEIFKSFPSVLGPLDENDPEGEEYNDDDDDDDDEDGDDANEDELQSKLKDLEIKQEE

SEQ ID NO: 97 - protein, 542 aa, Manihot esculenta
MDSTTQSFQHRTLHVKLWPPTQSTRLMLVERMTKNLITPSIISRKYGILSKEEAEEDARQIEESAFAAADQHHQKEPDGDGSSAVQVYAKESSKLMLEVLKRGPRMKEDGEAAVAEISAAAHADLFDLSGGRRAFISAEEANDLLKPLKEPGNTYTKICFSNRSFGLDAAGVAEPILSSIKDQLTEVDLSDFVAGRPEPEALQVMNIFSSALEGCNLRYLNLSNNALGEKGVRAFGALLRSQNNLEELYLINDGISEEAARAVCELIPSTEKLKVLQFHNNMTGDEGAVAIAEIVKRSAMLEDFRCSSTRIGSEGGVVLAEALGTCSHMRKLDLRDNMFGVEAGVALSKSLVAFADLTEVYLSYLNLEDDGAEALANALKESAPSLEVLEMAGNDITAKGASSLAACIAAKQFLSKLNLSENELKDEGAILIAKAIESGHGQLSEVDLSTNSIRRAGARLLGQAVVLKPGFKLLNINGNFISDEGIDELKDIFKNLPGVLGPLDDNDPEGEDLDEEAKEEDADNEDDLESKLKGLEIKHDEQ

SEQ ID NO: 98 - protein, 533 aa, Medicago sativa
MDSSVPSYQHRTLSIKLWPPSQSTRLMLVERMVRNLTTPSIFSRKYGLLNKDEAEKDAKEIEDAAFVTASQHFEKEPDGDGSSAVQIYAKESSKLMLEVLKRGPRGKEENGELISEKGDAAVETVFDISGGRRAFIDGQEASELLKPLMGPNSFTKICFSNRSFGLDAAHVVEPMLISIKDQLKEVDLSDFIAGRPEAEAIEVMNIFSSALERAVLRYLNLSNNAMGEKGVRAFRALLKSQNDLEELYLMNDGISEEAAKAVAELIPSTEKLKVLHFHNNMTGDEGAFAIAEVVKRSPALEDFRCSSTRVGSEGGVALAEALGACTHLKKLDLRDNMFGVEAGVALSKVIPVFADLTEIYLSYLNLEDDGAEALANALKESAPSLETLDMAGNDITAKATVSVAECISSKQFLTKLNLSENELKDEGAGLISKALEGRGQLSEVDLSTNLITWSGAKLLAEAVVQKPGFKLLNINANFISDEGIDELKDIFKNSPDMLGPLDENDPEGEDVDEEAEDDSDNDELESKLKGLEI

SEQ ID NO: 99 - protein, 534 aa, Nicotiana tabacum
MDSAGCSIKLWPPSLSTRLMLVEKMTKNLITPSILSRKYGLLSKEEAEEDAKQIEAVAFASANQHIDKEPDGDGSSAVQLYAKESSRLMLEVIKRGPQTKESAEDVIPEKVKPSDETTIFDLSKGRRDFINVEEADELLKPLSEPGNNYKRICFSNRSFGVDAAEIAGPILSSLKDQLTEVDLSDFIAGRPEVEALEVMEIFSSALGACDLRYLNLSNNALGEKGVRAFGALLKSQRNLEELYLMNDGISEEAAQAVCELIPSTDKLRILHFHNNMTGDEGALAISELVKHSPALEDFRCSSTRVGSEGGVALSQALGECRNLKKLDLRDNMFGVEAGIALSKVLSIFSDLTEIYLSYLNLEDEGSIALANALKGSAPSLEVLEMDGNDITAKAAPVMAACIAAKQFLTKLRLAENELKDEGAILIAKALEDGHGQLTELDMSTNAIRRAGARCLAQAVVSKPGFKVLSINGNFISDEGIDEVKDIFKNSLNVLGPLDENDPEGEDYDEEADEEADNEYELETRLKGLDIKQEE

SEQ ID NO: 100 - protein, 555 aa, Panicum virgatum
MDSAAQNFQPRTFSIKLWPPSESTRLKLVERMTKNLSTESIFSRKYGLLGKEEAHENAERIEQLCFGSADEHFKKEPDGDGSSAVQLYAKETSKMMLEVLKKGPRITAELEASVADTPLAPGDTVLDISGGKRAYIEADEAKQLLSPLTKPGNSYKRICFSNRSFGVDAANVAGSILESVKNQLTEVDISDFVAGRPEDEALDVMRIFSKALEGSVLRYLNISDNALGEKGVRAFSELLKSQENLEELYVMNDGISEDAAKALSELIPSTEKLKVLHFHNNMTGDEGAVYVAEMVKRSPNLESFRCSATRIGSDGGVALSEALGTCTHLKKLDLRDNLFGVDAGIALSKTLPKLPDLVELYLSDLNLENKGTVAIVNALKQSAPQLEVLELAGNEINAKAAPDLAECLTAMQSLKKLTLAENELKDAGAVIIAKSLEDGHVDLKEFDVSTNMFQRAGARCFARAVANKPGFVQLNIDGNFISDEGIDEVKDILKAGKNSLDVLGSLDENEPEGQPDDGDEDDDDDEGAKDDDVEDRLDSKLQNVQVEQDDEPCLG

SEQ ID NO: 101 - protein, 558 aa, Sorghum bicolor
MDPAQYFQPRTFSIKLWPPSESTRLMLVERMTKNLSTESESIFSRKYGTLGKEEAHENAKRIEELCFASADEHFKREPDGDGSSAVQLYAKETSKMMLEVLKKGPRTTAELEAPVADTPLVPDTPLVPADTPLVPADTVLDISGGKRAFIEADEAKELLSPLTKPGNSYQRICFSNRSFGIGAANVAGPILESVKNQLTEVDISDFVAGRPEDEALDVMRIFSKALEGSVLRYLNISDNALGEKGVRAFSELLKSQESLEELYVMNDGISEEAAKALSELIPATEKLKVLHFHNNMTGDEGAMYIAEMVKRSPNVESFRCSATRIGSDGGVALSEALGTCTRLKKLDLRDNLFGVDAGLALSKTLPKLPDLVELYLSDLNLENEGTIAIVKALKQSAPQLEVLEMAGNEINAKAAPDLAECLTAMQSLKKLTLAENELKDGGAVIIAKSLEDGHTDLKELDVSTNMLQRVGARCFARAVADKPAFVQLNINGNFISDEGIDEVKEILKAGKKSLDVLGSLDENEPDGEPDDEEEDEDAEDNEDELDSKLQSVKVEQDD

SEQ ID NO: 102 - protein, 534 aa, Solanum lycopersicum
MDSAGFSMKLWPPSSSTRLMLVERMTKNLITPSILSRKYGLLSKEEAEEDAKQIEALAFDSANQHFDKEPDGDGSSAVQLYAKESSKLMLEVIKRGPQTKESAEGIVSEKVKASNETTIFDISKGRRDFISAEEASELLKPLSEPGNNYKRICFSNRSFGGDAAKIAGPILSSLKDQLTEVDLSDFVAGRPEEEALEVMEIFSSALDACDLRYLDLSNNALGEKGIRAFGALLKSQKNLEELYLMNDGISEEAAEAVCELIPSTDKLRILHFHNNMTGDEGALSISKLVKHSPALEDFRCSSTRVGSEGGVALSQALGECRNLKKVDLRDNMFGVEAGIALSKVLSIFSGLTEIYLSYLNLEDEGSIALANVLRESAPSLEVLEMDGNDITAKAAPALAACIAAKQFLTTLKLGENELKDEGAILIAKALEDGHGQLTEVDMSTNAIRRAGARCLAQAVVNKPGFKVLNINGNFISDEGIDEVKDIFKNSLHVLGPLDDNDPEGEDYDEEADEGGDNENDLETRLKDLDIKQEE

SEQ ID NO: 103 - protein, 555 aa, Triticum aestivum
MESTAEGLQPRTFSVKLWPPSESTRLMLVERMTKNLSAECIFSRKYGILSKEEAHENAKRIEEVCFASAEEHFKKEPDGDGSSAVQLYAKETSKLMLEVLKKGPRTTEEPEAPVVDTPLEPADTVFDISGGKRAFIEAEEAKELLSPLTKPGNSYKRICFSNRSFGIGAANVAGPILESIKSQLTEVDISDFVAGRPEDEALDVMRIFSKALAGSVLSYLNISDNALGEKGVRAFTELLKSQGDLEELYVMNDGISEEAAKALSELIPSTEKLKVLHFHNNMTGDEGAMPIAEMVKRSPNLESFRCSATRIGSDGGVALAEALGTCTRLKKLDIRDNLFGVEAGVALSKTLPKLGGLVELYLSDLNLENEGTIAIVNVLKQSAPQLEVLEMAGNEITAKAAKAVAECLTAMQSLKKLTLAENELKDDGAVTIAKSLQEGHPDLKELDVSTNLFQRSGARCFAQAVTNKPGFVLLNINSNFISNKGVDEVKEILKGGKNSLEVLGPLDENDPEGDPEDGEDEEDGEDADDEEKDGDDNGDGGLGSKLQGLKVEEED

SEQ ID NO: 104 - protein, 537 aa, Theobroma cacao
MESGSQALQHRLLSVKLWPPSYSTRLMLVERMTKNLTTPSIFSRKYGLLSSEEAEEDAKKIEELAFATADQHYKKEPDGDGSSAVQIYAKESSRLMLEVIKRGPTVKDGELADKNTALHETVFDISGGRRAFIDAEEAEDLLKPLRQPGNSYTKICFSNRSFGLDAANVAAPILSSIKDQLTEVDLSDFIAGRPESEALEVMNIFSSVLEGCHLRYLNLSNNALGEKGVRAFGALLKSQNNLEELYLMNDGISEEAARAVSELIPSTEKLKVLQFHNNMTGDEGAIAISEILKRSCALEDFRCSSTRVGADGGVALAEALKTCTHLKKLDLRDNMFGVEAGVALSEALSLFANLTEVYLSFLNLEDEGAEALANALKESAPSLEVLEMAGNDITAKGAASLAACIASKQFLTKLNLAENELKDEGAILIAKAIGEGHGQLNEVDMSSNAIRRAGARLLAQVVVKKPGFKLLNINGNFISDDGIDEVKESFKSWPDMLGPLDENDPEGEDDEDAEEENAENESELESKLKDLKIEQEE

SEQ ID NO: 105 - protein, 532 aa, Trifolium subterraneum
MDSTVQSYPHRSLSIKLWPPSQSTRLMLVERMTKNLTTPSIFSRKYGLLSKEEAEVDAKEIEDAAFVTATQHFEKEPDGDGSSAVQIYAKESSKLMLEVLKRGPRRKEDGELVSEKAGATVETVFDISGGRRAFIDKEEASELLKPLMAPSSFTKICFSNRSFGLEAANVAGPILISIKDQLKEVDLSDFIAGRPEAEALEVMNIFSSALEGSVLRYLNLSNNAMGEKGVRAFRSLLKSQNDLEELYLMNDGISEEAAKAVAELIPSTEKLKVLHFHNNMTGDEGAFAIAEVVKRSPALEDFRCSSTRVGSEGGVALAEALGACTHLKKLDLRDNMFGVEAGVALSKVIPLFVDLTEIYLSYLNLEDEGAEALANALKESAPSLEILDMAGNDISATAAVSVAACVSSKQFLTKLNLSENELKDEGAALISKALEGGHGQLNEVDLSTNLITWSGAKLLAEAVVQKPGFKLLNINANFISDDGIDELKKIFKNSPDMLGPLDENDPEGEDIDEEAEDSDNDELESKLKGLGI

SEQ ID NO: 106 - protein, 532 aa, Vigna unguiculata
MDSSAQAYQHRPLAIKLWPPSQGTRLMLVERMTKNLTTPSIFSRKYGLLSKEEAEEDAKQIEDDAFATATQHFEKEPDGDGSSAVQIYAKESSKLMLEVLKRGPRVKEGGELTSGKADATAETVFDISGGRRAFIEGKEAAELLEPLRGPNSYTKIIFSNRSFGLDAARVAEPILISIKDQLKEVDLSDFIAGRTEAEALEVMSIFSSALEGCALRYLNLSNNAVGEKGVRAFRSLLKSQINLEELYLMNDGISEEAAKAVSELLPSTEKLRVLHFHNNMTGDEGAIAIAEIVKHSPALEDFRCSSTRVGSDGGVALAEALGACKHLRKLDLRDNMFGAEAGVALSKVIPAYTDLTEIYLSYLNLEDDGAEALANALKESAPSLEILDLAGNDITAEGAVSVAACISSKQFLTKINLSENELKDEGVALISKALEVGRQLIEVDLSTNSITWSGAKLVAEAVVGKPGFKLLNINANFISDEGIVELKNIFKNSPDMLGPLDENDPEGEDNEGKDEEDGEHDELESKLKGLGI

SEQ ID NO: 107 - protein, 516 aa, Zea mays
MLVDRMTNNLSTESIFSRKYRLLGKQEAHENAKTIEELCFALADEHFREEPDGDGSSAVQLYAKETSKMMLEVLKKGPRTTAELEAPVADTPLVSADTVLDISGGKRAFIEADEAKELLSPLTKPGNSYKRICFSNRSFGIGAANVAGPILESVKNQLTEVDISDFVAGRSEDEALDVMRIFSKALDGSVLRYLNISDNALGEKGVRAFSELLKSQESLEELYVMNDGISEEAAKALSELIPATEKLKVLHFHNNMTGDEGAMYIAEMVKRSPNVESFRCSATRIGSDGGVALSEALGTCTRLKKLDLRDNLFGVDAGLALSETLPKLPDLVELYLSDLNLENKGTIAIAKALKQSALQLEVLEIAGNEINAKAAPDLAECLAVMQSLKKLTLAENELKDNGAVIIAKSLEDGHSDLKELDVSTNMLQRVGARCFARAVANKPDFVQLNINGNFISDEGVDEVKEILKAGKKSLDVLGPLDENEPDGEPDDEDAEDDEDELDLNLQSVKVEQDEDD

SEQ ID NO: 108 - DNA, 1608 bp, Arabidopsis thaliana
  source 1..1608: mol_type = genomic DNA
atggatcattcggcgaaaacgacacaaaatcgtgtattgtccgtcaaaatgtggccgccttcgaaatcaaccaggctgatgttggtggagagaatgacaaagaatatcacaacaccttcaatcttctctcgtaagtacggacttcttagcgtagaggaggccgaacaagacgctaaaagaattgaggatcttgccttcgcaaccgccaacaagcatttccagaacgaaccggatggtgacggcacgtccgcggtgcacgtgtatgcaaaggagagctcaaaattgatgttggacgttataaagaggggcccccaagaagagtccgaagtcgaagtcagtaaagatggcgacgtcttttttgatatttctggaggaagccgtgcatttatcgaggaagaggaagcgagagatttgctcaggcccctggcagacccgcgtaatagttatactaagataagattcagtaataggtcctttggctctgaagccgcaaagttcgcagctagcgttctttcttcaatcaaagatcaactcacagaggtcgatttgagtgatttcgttgcggggagaccagaagcggaagcacttgaggtaatgaacatgttttcttccgcgctggaaggaagcaaattgaggtatttgaacctttctgacaacgcgcttggtgagaagggcatcagagcatttgccagtctcataaacagtcagcacgatttggaggaactttaccttatgaacgatggtatatcagaggatgcagccagagctgtcagggaattgttgccatctactgacaagattagggtacttcaatttcataacaacatgaccggggacgaaggagcgacagccattgcagaaatcgtacgtgagtgtccgtctctcgaggatttcaggtgtagcagcacaaggatcggttctgaaggaggtgtcgccctggctgaagcgctggagcactgttcacatttgaagaagttggatcttagagataatatgtttggtgtagaaggtggaattgcactcgcgaagacactttccgtcctcacccacctcacggaaatctacatgtcgtacttgaatctggaagacgaggggaccgaggcactttctgaggccctgctcaagtccgctccttctttggaggtactggagctggcgggcaacgatattacagtaaagtcgaccggtaatcttgcggcctgtatcgcatccaagcagtcgcttgctaaactcaatctctctgagaacgagttgaaggacgaaggcactattctcattgctaaagctgtcgaggggcatgatcaactggtcgaggttgatctttccaccaatatgataaggagggccggagctagggcgctggcgcagactgtagttaaaaagaatactttcaagctcctcaatataaatggcaatttcatcagtgaggagggcatcgatgaagtaaacgacatgtttaaggattgcctcgacaaacttgtgccgcttgatgataacgacccagaaggcgaagacttcgaggacgaggacgaagaagaggaaggagaagatggtaacgagcttgagtcaaagctggggagccttaagataaaacaaggtgaggaatag

SEQ ID NO: 109 - DNA, 1245 bp, Arabidopsis thaliana
  source 1..1245: mol_type = genomic DNA
atgtctggaggaagccgtgcatttatcgaggaagaggaagcgagagatttgctcaggcccctggcagacccgcgtaatagttatactaagataagattcagtaataggtcctttggctctgaagccgcaaagttcgcagctagcgttctttcttcaatcaaagatcaactcacagaggtcgatttgagtgatttcgttgcggggagaccagaagcggaagcacttgaggtaatgaacatgttttcttccgcgctggaaggaagcaaattgaggtatttgaacctttctgacaacgcgcttggtgagaagggcatcagagcatttgccagtctcataaacagtcagcacgatttggaggaactttaccttatgaacgatggtatatcagaggatgcagccagagctgtcagggaattgttgccatctactgacaagattagggtacttcaatttcataacaacatgaccggggacgaaggagcgacagccattgcagaaatcgtacgtgagtgtccgtctctcgaggatttcaggtgtagcagcacaaggatcggttctgaaggaggtgtcgccctggctgaagcgctggagcactgttcacatttgaagaagttggatcttagagataatatgtttggtgtagaaggtggaattgcactcgcgaagacactttccgtcctcacccacctcacggaaatctacatgtcgtacttgaatctggaagacgaggggaccgaggcactttctgaggccctgctcaagtccgctccttctttggaggtactggagctggcgggcaacgatattacagtaaagtcgaccggtaatcttgcggcctgtatcgcatccaagcagtcgcttgctaaactcaatctctctgagaacgagttgaaggacgaaggcactattctcattgctaaagctgtcgaggggcatgatcaactggtcgaggttgatctttccaccaatatgataaggagggccggagctagggcgctggcgcagactgtagttaaaaagaatactttcaagctcctcaatataaatggcaatttcatcagtgaggagggcatcgatgaagtaaacgacatgtttaaggattgcctcgacaaacttgtgccgcttgatgataacgacccagaaggcgaagacttcgaggacgaggacgaagaagaggaaggagaagatggtaacgagcttgagtcaaagctggggagccttaagataaaacaaggtgaggaatag

SEQ ID NO: 110 - DNA, 729 bp, Arabidopsis thaliana
  source 1..729: mol_type = genomic DNA
atgctcgaggatttcaggtgtagcagcacaaggatcggttctgaaggaggtgtcgccctggctgaagcgctggagcactgttcacatttgaagaagttggatcttagagataatatgtttggtgtagaaggtggaattgcactcgcgaagacactttccgtcctcacccacctcacggaaatctacatgtcgtacttgaatctggaagacgaggggaccgaggcactttctgaggccctgctcaagtccgctccttctttggaggtactggagctggcgggcaacgatattacagtaaagtcgaccggtaatcttgcggcctgtatcgcatccaagcagtcgcttgctaaactcaatctctctgagaacgagttgaaggacgaaggcactattctcattgctaaagctgtcgaggggcatgatcaactggtcgaggttgatctttccaccaatatgataaggagggccggagctagggcgctggcgcagactgtagttaaaaagaatactttcaagctcctcaatataaatggcaatttcatcagtgaggagggcatcgatgaagtaaacgacatgtttaaggattgcctcgacaaacttgtgccgcttgatgataacgacccagaaggcgaagacttcgaggacgaggacgaagaagaggaaggagaagatggtaacgagcttgagtcaaagctggggagccttaagataaaacaaggtgaggaatag

SEQ ID NO: 111 - DNA, 690 bp, Arabidopsis thaliana
  source 1..690: mol_type = genomic DNA
atggaaggaggtgtcgccctggctgaagcgctggagcactgttcacatttgaagaagttggatcttagagataatatgtttggtgtagaaggtggaattgcactcgcgaagacactttccgtcctcacccacctcacggaaatctacatgtcgtacttgaatctggaagacgaggggaccgaggcactttctgaggccctgctcaagtccgctccttctttggaggtactggagctggcgggcaacgatattacagtaaagtcgaccggtaatcttgcggcctgtatcgcatccaagcagtcgcttgctaaactcaatctctctgagaacgagttgaaggacgaaggcactattctcattgctaaagctgtcgaggggcatgatcaactggtcgaggttgatctttccaccaatatgataaggagggccggagctagggcgctggcgcagactgtagttaaaaagaatactttcaagctcctcaatataaatggcaatttcatcagtgaggagggcatcgatgaagtaaacgacatgtttaaggattgcctcgacaaacttgtgccgcttgatgataacgacccagaaggcgaagacttcgaggacgaggacgaagaagaggaaggagaagatggtaacgagcttgagtcaaagctggggagccttaagataaaacaaggtgaggaatag

SEQ ID NO: 112 - DNA, 1530 bp, Arabidopsis thaliana
  source 1..1530: mol_type = genomic DNA
atgttggtggagagaatgacaaagaatatcacaacaccttcaatcttctctcgtaagtacggacttcttagcgtagaggaggccgaacaagacgctaaaagaattgaggatcttgccttcgcaaccgccaacaagcatttccagaacgaaccggatggtgacggcacgtccgcggtgcacgtgtatgcaaaggagagctcaaaattgatgttggacgttataaagaggggcccccaagaagagtccgaagtcgaagtcagtaaagatggcgacgtcttttttgatatttctggaggaagccgtgcatttatcgaggaagaggaagcgagagatttgctcaggcccctggcagacccgcgtaatagttatactaagataagattcagtaataggtcctttggctctgaagccgcaaagttcgcagctagcgttctttcttcaatcaaagatcaactcacagaggtcgatttgagtgatttcgttgcggggagaccagaagcggaagcacttgaggtaatgaacatgttttcttccgcgctggaaggaagcaaattgaggtatttgaacctttctgacaacgcgcttggtgagaagggcatcagagcatttgccagtctcataaacagtcagcacgatttggaggaactttaccttatgaacgatggtatatcagaggatgcagccagagctgtcagggaattgttgccatctactgacaagattagggtacttcaatttcataacaacatgaccggggacgaaggagcgacagccattgcagaaatcgtacgtgagtgtccgtctctcgaggatttcaggtgtagcagcacaaggatcggttctgaaggaggtgtcgccctggctgaagcgctggagcactgttcacatttgaagaagttggatcttagagataatatgtttggtgtagaaggtggaattgcactcgcgaagacactttccgtcctcacccacctcacggaaatctacatgtcgtacttgaatctggaagacgaggggaccgaggcactttctgaggccctgctcaagtccgctccttctttggaggtactggagctggcgggcaacgatattacagtaaagtcgaccggtaatcttgcggcctgtatcgcatccaagcagtcgcttgctaaactcaatctctctgagaacgagttgaaggacgaaggcactattctcattgctaaagctgtcgaggggcatgatcaactggtcgaggttgatctttccaccaatatgataaggagggccggagctagggcgctggcgcagactgtagttaaaaagaatactttcaagctcctcaatataaatggcaatttcatcagtgaggagggcatcgatgaagtaaacgacatgtttaaggattgcctcgacaaacttgtgccgcttgatgataacgacccagaaggcgaagacttcgaggacgaggacgaagaagaggaaggagaagatggtaacgagcttgagtcaaagctggggagccttaagataaaacaaggtgaggaatag